(19) United States
(12) Patent Application Publication
(10) Pub. No.: US 2006/0024350 A1
(43) Pub. Date: Feb. 2, 2006

(54) Biodegradable ocular devices, methods and systems

(76) Inventors: Signe E. Varner, Los Angeles, CA (US); Patrick E. Guire, Eden Prairie, MN (US); Kristin S. Taton, Little Canada, MN (US); Jie Wen, Eden Prairie, MN (US); Nathan R.F. Beeley, Irvine, CA (US); Laurie R. Lawin, New Brighton, MN (US); Robert W. Hergenrother, Eden Prairie, MN (US); Ralph A. Chappa, Prior Lake, MN (US); Aron B. Anderson, Minnetonka, MN (US)

Correspondence Address: KARRIE WEAVER;Kagan Binder, PLLC, Suite 200, 221 Main Street North, Stillwater, MN, 55082, US

(21) Appl. No.: 11/165,884

(22) Filed: Jun. 24, 2005

Related U.S. Application Data
(60) Provisional application No. 60/583,171, filed on Jun. 24, 2004.
(60) Provisional application No. 60/669,701, filed on Apr. 8, 2005.

(51) Int. Cl. A61F 2/00

(52) U.S. Cl. 424/427

(57) ABSTRACT

The invention provides implantable medical devices that are fabricated of biodegradable materials for delivery of bioactive agent to limited access regions of a patient's body, such as the eye. The invention further provides methods of treatment utilizing the devices.

This application claims the benefit of U.S. Provisional Application Ser. No. 60/583,171, filed Jun. 24, 2004, entitled “BIODEGRADABLE MEDICAL DEVICE,” and U.S. Provisional Application Ser. No. 60/669,701, filed Apr. 8, 2005, entitled “SUSTAINED DELIVERY DEVICES FOR THE CHOROID AND RETINA AND METHODS FOR SUBRETINAL ADMINISTRATION OF BIOACTIVE AGENTS TO TREAT AND/OR PREVENT RETINAL DISEASES,” which applications are incorporated herein by reference in their entirety.

### FIELD OF THE INVENTION

The invention relates to medical devices having a biodegradable component that are useful for effectively treating a treatment site within a patient's body, for example, treatment of limited access regions (such as the eye) within the body.

### BACKGROUND OF THE INVENTION

The use of implantable devices for delivery of drugs to specific sites within the body is a relatively new and exciting realm of medical science. However, placement of implantable devices in limited access regions of the body can present challenges. Limited access regions of the body can be characterized in terms of physical accessibility as well as therapeutic accessibility. Factors that can contribute to physical accessibility difficulties include the size of the region to be reached (for example, small areas such as glands), the location of the region within the body (for example, areas that are embedded within the body, such as the middle or inner ear), the tissues surrounding the region (for example, areas such as the eye or areas of the body surrounded by highly vascularized tissue), or the tissue to be treated (for example, when the area to be treated is composed of particularly sensitive tissue, such as areas of the brain).

Factors that can contribute to therapeutic accessibility can be seen, for example, in the delivery of drugs to the eye. Ocular absorption of systemically administered pharmacologic agents is limited by the blood ocular barrier, namely the tight junctions of the retinal pigment agents is limited by the blood ocular barrier, namely the tight junctions of the retinal pigment epithelium and vascular endothelial cells. High systemic doses of bioactive agents can penetrate this blood ocular barrier in relatively small amounts, but expose the patient to the risk of systemic toxicity. Intravitreal injection of bioactive agents (such as drugs) is an effective means of delivering a drug to the posterior segment of the eye in high concentrations. However, these repeated injections carry the risk of such complications as infection, hemorrhage, and retinal detachment. Patients also often find this procedure somewhat difficult to endure.

A number of techniques or methodologies have been developed to deliver drugs to the various tissues or structures that make up the mammalian eye to treat a wide range of disorders or diseases of the eye. However, delivery of drugs, proteins and the like to the eye(s) of mammals so as to achieve the desired therapeutic or medical effect, especially to the retina and/or the choroid, has proven to be challenging, particularly as a result of the geometry, delicacy and/or behavior of the eye and its components.

For example, oral ingestion of a drug or injection of a drug at a site other than the eye can provide a drug systemically, however, such a systemic administration does not provide effective levels of the drug specifically to the eye. In many ophthalmic disorders involving the retina, posterior tract, and optic nerve, adequate levels of the drug cannot be achieved or maintained by oral or parenteral routes of administration. Thus, further and repeated administration of the drug would be necessary to achieve the desired or adequate levels of concentration of the drug. Such further and repeated administrations of such drugs, however, may produce undesired systemic toxicity.

Ophthalmic conditions have also been treated using drugs applied directly to the eye in either liquid or ointment form. This route of administration (topical administration), however, is only effective in treating problems involving the superficial surface of the eye and diseases that involve the cornea and anterior segment of the eye, such as for example, conjunctivitis. Topical administration of drugs is ineffective in achieving adequate concentrations of a drug(s) in the sclera, vitreous, or posterior segment of the eye. In addition, topical eye drops may drain from the eye through the nasolacrimal duct and into the systemic circulation, further diluting the medication and risking unwanted systemic side effects. Furthermore, delivery of drugs in the form of topical eye drops is also of little utility because the drug cannot cross the cornea and be made available to the vitreous, retina, or other subretinal structures such as the retinal pigment epithelium (“RPE”) or choroidal vasculature. Typically, drugs of interest are highly unstable and therefore not easily formulated for topical delivery. Moreover, data also indicates that it is not unusual for up to 85% of topically applied agents to be removed by the eye's blink mechanism/reflex.

Direct delivery of drugs to the eye by a topical insert has also been attempted, however, this method is not desirable. Such topical inserts require patient self-administration and thus education on their insertion into and removal from the eye. Consequently, this technique demands a certain degree of manual dexterity that can be problematic for geriatric patients who are particularly susceptible to certain eye disorders that appear age related (such as age related macular degeneration). Also, in many instances such topical inserts may cause eye irritation and such inserts are prone to inadvertent loss due to eyelid laxity. In addition, these devices provide a source of drug only to the cornea and anterior chamber, and thus do not provide any pharmacologic advantage over topical eye drops or ointments. Thus, such devices have limited, if any at all, utility for providing an effective source of drugs to the vitreous or tissues located in the posterior segment of the eye.

As a consequence, most methods for treating eye disorders or diseases in the posterior segment, or the back-of-the-eye, involve intravitreal delivery of the drug. One such technique for intravitreal delivery is accomplished by intraocular injection of the drug or microspheres containing the drug directly into the vitreous or by locating a device or capsule containing the drug in the vitreous, such as that described in U.S. Pat. No. 5,770,589. Intravitreal injection of a drug is an effective means of delivering the drug to the posterior segment of the eye in high concentrations, but it is not without its shortcomings. It is well known that drugs that are initially located within the vitreous are cleared over time.

In addition, it also is well known that many therapeutic drugs cannot easily diffuse across the retina. Thus, the dose being administered and maintained in the vitreous has to take into account the amount that can diffuse across the retinal boundary as well as how long the drug is retained in effective amounts within the vitreous. For example, it has been observed from animal studies that 72 hours after injection of triamcinolone, less than 1% of the triamcinolone present in the vitreous was associated with other tissues including the retina, pigment epithelium, and sclera. In addition to the relative effectiveness of drug delivery across the barrier, complications or side effects have been observed when using the direct injection into vitreous technique with some therapeutics.

For example, compounds classified as corticosteroids, such as triamcinolone, can effectively treat some forms of neovascularization such as corneal neovascularization. When these compounds were used to treat neovascularization of the posterior segment by direct injection, these compounds were observed to cause undesirable side effects in many patients. The adverse affects or undesirable side effects being observed included elevations in intraocular pressure and the formation of, or acceleration of the development of cataracts. Elevations in intraocular pressure are of particular concern in patients who are already suffering from elevated intraocular pressure, such as glaucoma patients. Moreover, a risk exists that the use of corticosteroids in patients with normal intraocular pressure will cause elevations in pressure that result in damage to ocular tissue. Since therapy with corticosteroids is frequently long term (typically several days or more), a potential exists for significant damage to ocular tissue as a result of prolonged elevations in intraocular pressure attributable to that therapy.

Consequently, efforts in the area of intravitreal delivery also have included delivery by locating a sustained release implant, capsule or other such device or mechanism that is in communication with the vitreous and which is configured so as to provide a release over time into the vitreous of the contained drug. Examples of such controlled release devices are described in U.S. Pat. Nos. 6,217,895; 5,773,019; 5,378,475; and in U.S. Patent Application Publication No. 2002/0061327.

A common feature of the techniques/instruments described therein, is that a surgical incision is required to be made at the outset of a procedure so that the implant, capsule or other such device can be inserted through the eye and located in the vitreous. These methods and techniques also necessarily involve the use of sutures following completion of the procedure to seal or close the incision so as to prevent loss of vitreous material. As is known to those skilled in the art, maintaining the volume of the posterior segment or vitreous is necessary to maintaining the shape and optical arrangement of the eye. Such a course of treatment also increases the duration and cost as well as the realistic risks of corneal ulceration, cataract formation, intraocular infection, and/or vitreous loss that accompany these procedures.

There is described in U.S. Pat. No. 5,516,522 a biodegradable porous drug delivery device for controllably releasing a pharmacological agent. The device comprises a hollow tube having an interior surface and an exterior surface and a first end and a second end. A pharmacological agent is filled into the hollow tube for controllable release through the channels of the tube. Prior to the pharmacological agent being filled into the hollow tube, the first end is heat sealed, and after the pharmacological agent is filled into the hollow tube, the second end is heat sealed.

Thus, there are a number of drawbacks with currently available methods for treating eye disorders and diseases. For example, in the case of these posterior segment eye diseases, traditional routes of drug administration such as topical or oral dosing often fall short of reaching the disease site. As a result, current methods for treating back-of-the-eye diseases involve introducing drugs directly into the vitreous chamber of the eye via intraocular injections or intravitreal implants. The eye's natural circulatory processes rapidly remove solutions that are injected directly into the vitreous chamber. Subsequently, this approach often requires frequent, large dose injections that have been associated with complications such as glaucoma and cataract formation. Furthermore, large molecular weight molecules (>70 kD) are virtually incapable of traversing the tight junction complexes of the retinal pigment epithelium and retinal capillaries. Microparticle injections have improved the sustained release capabilities of conventional injections, but this still does not resolve the widespread distribution of the medication via intraocular convection. In the case of steroids, this distribution is known to lead to adverse effects such as glaucoma and cataract. Additionally, the eye's natural circulatory processes have a subtle anterior to posterior ocular convection, which results in lower drug concentrations at the back of the eye where the disease is developing.

Other sustained delivery devices for intraocular application are described in U.S. Pat. No. 6,719,750 B2 (“Devices for Intraocular Drug Delivery,” Varner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and related applications.

Ophthalmic devices for subretinal application include, but are not limited to, those described in U.S. Patent Publication No. 2002/0198511 A1 (“Method and Device for Subretinal Drug Delivery,” Varner et al.), and PCT Publication No. WO 2004/028477 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); and related applications.

### SUMMARY OF THE INVENTION

Generally, the invention provides implantable medical devices fabricated from biodegradable or bioresorbable materials. The implantable devices find particular application for delivery of one or more bioactive agents to limited access regions of the body. In some aspects, the inventive devices and methods allow for site-specific delivery of bioactive agent to areas of the body. Illustrative limited access regions include eye, ear, sinuses, central nervous system (spinal cord, brain), joints, and the like.

In some aspects, the polymeric formulations of the invention biodegrade within a period that is acceptable for the desired application. In certain aspects, such as in vivo therapy, such degradation occurs in a period usually less than about three years, or less than about two years, or less than about one year, or less than about six months, three months, one month, fifteen days, five days, three days, or even one day, on exposure to a physiological solution with a pH between 6 and 8 having a temperature in the range of about 25° to about 37° C. In some embodiments, the polymeric formulation of the invention degrades in a period in the range of about an hour to several weeks, depending upon the desired application.

In its article aspects, the invention provides an implantable medical device for delivering one or more bioactive agents to limited access regions of the body, the implant comprising one or more solid biodegradable polymers and one or more bioactive agents, wherein the one or more biodegradable polymers form a polymer matrix including the one or more bioactive agents. The implant delivers the one or more bioactive agents in an amount substantially less than the amount delivered by systemic, topical, and whole organ delivery systems in order to achieve the same therapeutic or prophylactic effect.

In some aspects, the biodegradable polymer comprises a biodegradable amphiphilic block copolymer comprising hydrophilic blocks and hydrophobic blocks. The inventive implants can be partially or completely biodegradable.

In some embodiments, the polymer matrix and the one or more bioactive agents, alone, form the implant. In other embodiments, the implant comprises a biocompatible core that is at least partially coated with a coating that comprises a biodegradable polymer matrix and one or more bioactive agents. In some embodiments, the coating covers the entire outer surface of the core. In other embodiments, the coating covers one or more portions of the outer surface of the core, leaving one or more portions of the outer surface of the core exposed.

In some embodiments the coating is tapered or feathered at one or both ends of the coating. In some embodiments at least the distal and proximal ends of the core are covered with coating and the coating is tapered or feathered. In other embodiments, an intermediate portion of the core is provided with a coating, and the proximal and/or distal ends of the core are uncoated. In these particular aspects, the coating can, in some embodiments, be tapered or feathered. In some embodiments, the core material and/or the core thickness is selected to provide the implant with a desired rigidity/flexibility. The core can comprise biodegradable or biostable materials.

The inventive implantable devices thus include a biodegradable component. Generally, at least the polymer matrix (which includes bioactive agent) includes a biodegradable polymer. In some aspects, the biodegradable polymer is an amphiphilic copolymer comprising hydrophilic blocks and hydrophobic blocks. Illustrative amphiphilic copolymers are composed of polyalkylene glycol blocks (hydrophilic) and aromatic polyester blocks (hydrophobic). In other aspects, the polymer material can be selected from materials that can be viewed (for purposes of discussion) as falling within two general groups. The first group can be thought of as polymers containing ester linkages, such as polyetherester copolymers, terephthalate esters with phosphorus-containing linkages, and segmented copolymers with differing ester linkages. A second group is composed of polycarbonate-containing random copolymers. In another aspect, copolymers and/or blends of any of the biodegradable polymers listed herein can be utilized. The polymer matrix includes one or more bioactive agents, thereby providing a drug-delivery device.

Additional representative examples of biodegradable polymers that could be used in forming the polymer matrix of an implant include poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L lactic acid), poly(glycolic acid-co-trimethylene carbonate), poly(phosphate esters), polyphosphoester urethanes, poly(amino acids), cyanoacrylates, poly(trimethylene carbonates), degradable polycarbonates, poly(iminocarbonates), polyesters, copoly(ether-esters), polyalkylene oxalates, polyphosphazenes and copolymers and blends of the above polymers. Biodegradable materials such as (but not limited to) cellulose, dextrans, polysaccharides, and hyaluronic acid could also be used. Blends including any two or more of these can be used.

As mentioned, the polymer matrix including bioactive agent can alone form the implant. In other embodiments of the invention, the implant can include a core. The core can be fabricated from a biodegradable or biostable material. Any of the biodegradable polymers described herein as suitable for the biodegradable polymer matrix can be utilized to form a core. In these aspects, the biodegradable polymer (or polymers) selected for the core can be the same or different from the biodegradable polymer (or polymers) selected for the polymer matrix.

When the core is formulated of a biostable material, the overall implant is considered partially degradable, in that the core will not be broken down by the body. Representative biostable polymers for forming the core include polyurethanes, silicones, polyesters, polyolefins (for example, polyethylene or polypropylene), polyisobutylene, acrylic polymers, vinyl halide polymers, polyvinyl ethers, polyvinyl methyl ether, polyvinylidene halides, polyacrylonitrile, polyvinyl ketones, polyvinyl aromatics, polyvinyl esters (for example, poly(alkyl(meth)acrylates) such as poly((methyl)methacrylate) or poly((butyl)methacrylate)), polyvinyl amides, polyamides, polycaprolactam, polycarbonates, polyoxymethylenes, polyimides, polyethers, polyurethanes, rayon, rayon-triacetate, cellulose acetate, cellulose butyrate, cellophane, cellulose nitrate, cellulose propionate, cellulose ethers, carboxymethyl cellulose and copolymers (for example, polyethylene vinyl acetate) and blends of any two or more of these polymers.

Non-polymer based biocompatible materials may also be used as the core of an implant of the invention. Representative examples include titanium-nickel alloy wire, titanium alloys, nickel-cobalt base alloys, stainless steel, cobalt-chromium alloys, and biodegradable magnesium alloys. In one embodiment, the core is titanium nickel wire having the smallest commercially available diameter, thereby maximizing the volume of bioactive agent(s) that the implant can contain.

In some aspects, the core is partially or totally covered with a coating comprising a biocompatible, biodegradable polymer matrix and one or more bioactive agents, and the coating is further provided with one or more additional coating layers of polymer material that modifies the release rate characteristics of the biodegradable polymer matrix. Exemplary polymers making up the additional layer(s) include polycaprolactone, poly(methylmethacrylate), polyesters, polyorthoesters, polypropylene, polyethylene vinyl acetate or poly(butylmethacrylate). In one embodiment, the layer of material is polycaprolactone.

In embodiments of the invention, the core is partially coated with a coating comprising a biocompatible, biodegradable polymer matrix and one or more bioactive agents, while the uncoated length of the core provides handling portions (for example, uncoated regions of about 10 mm in length, or less) by which the implant may be grasped, docked with a surgical instrument, or used for easy device retrieval after a period of implantation in the eye. Further, one or more portions of the coating may be tapered or feathered, and in some embodiments, at least the distal and proximal ends of the core are coated and the coatings are tapered or feathered.

Optionally, the core can include one or more bioactive agents. The bioactive agent included in the core can be the same, or different from, any bioactive agent included in the polymer matrix. Typically, but not always, bioactive agent included in the core is released during a period subsequent to release of the bioactive agent from the coating. Alternatively, when it is desired to release bioactive agent from the core during a time period that at least overlaps with a portion of the period of release of bioactive agent from the coating, it can be desirable to select the bioactive agents and polymeric coating materials to allow diffusion of the bioactive agent from the core and through the coating material. When included, a coating can be provided on the entire surface of the core, or substantially the entire surface. In other aspects, a coating can be provided on a selected portion of the core surface. For example, a coating can be provided at an intermediate portion of the core surface only. In still further aspects, more than one bioactive agent can be included in the coating.

In a more specific aspect, the invention provides devices and methods for providing treatment of limited access regions within the body, such as the eye, wherein the devices include at least a component that is biodegradable and/or bioerodable. In preferred aspects, any portions of the device that remain in the body (are not degraded and/or resorbed) do not cause significant adverse foreign body response and/or do not interfere with normal function of the area of the body at which the implant is located (for example, visual function within the eye). In some method aspects, the invention provides methods of making a device for controlled release of bioactive agent to a limited access region of the body, the method comprising steps of providing a biodegradable amphiphilic block copolymer comprising hydrophilic blocks and hydrophobic blocks, and forming the copolymer into an implant configured for placement in ocular tissues within the posterior region of the eye. Generally, the posterior region of the eye is understood to refer to regions behind the lens (as opposed to the anterior region of the eye, which is in front of the lens). In some aspects, the implant can be configured for placement in a subretinal area, an intraocular region, or other desirable tissue. In some embodiments, the implant comprises a filament, rod, C-shaped implant, coil, film, ribbon, block, disc, or pellet for placement in a subretinal area of the eye. In some embodiments, the implant comprises a nonlinear body member having a direction of extension, a longitudinal axis along the direction of extension, and a proximal end and a distal end, wherein at least a portion of the body member deviates form the direction of extension, the implant configured for placement in a vitreous area of the eye. The implant can be fabricated of polymer matrix (biodegradable polymer with bioactive agent) alone, or can include a core. When a core is included, the polymer matrix is provided as a coating on a surface of the core.

In further aspects, the invention provides methods for delivery of bioactive agent to limited accession regions within a patient in a controlled manner, the method comprising steps of implanting a device in a posterior region of the patient's eye, the device comprising a body member fabricated of a biodegradable amphiphilic block copolymer comprising hydrophilic blocks and hydrophobic blocks. In some aspects, the method further includes a step of allowing the device to remain in the patient for a selected period of time, wherein the device is configured to degrade upon implantation for a degradation period, and wherein bioactive agent is released in a controlled manner for a release period, the release period constituting at least a portion of the degradation period. Generally, the degradation period is longer than the bioactive agent release period. In some aspects, the release period comprises 50% or less of the degradation period. In some aspects, the degradation period is 3 years or less, or 2 years or less, or in the range of 0.5 to 2 years.

The inventive devices are formulated and configured to provide bioactive agent release to a treatment site for a treatment course. If desired, the implants of the invention can be removed from the implantation site after a desired treatment has been accomplished. It is understood that the inventive devices are generally removable from a patient at any time by an interventionalist. Such removal can be accomplished at the conclusion of a treatment course, or before completion of the treatment course, or after completion of the treatment course. In some aspects, an interventionalist can decide to remove the implant after a period of time that is shorter than an original anticipated implantation period. It is understood that removal of the inventive devices are not required, however, particularly when significant portions of the device (or even the entire device) are degradable.

Generally speaking, the inventive bioactive agent delivery systems can provide a controlled release profile of bioactive agent from the biodegradable implantable devices. The release profile is the cumulative mass of bioactive agent released versus time. The time profile of the release of bioactive agent, including immediate release and subsequent, sustained release can be predictably controlled utilizing the inventive compositions and methods. In preferred aspects, the initial release of bioactive agent is controlled, thereby permitting more of the bioactive agent to remain available at later times for a more extended release duration. The shape of the release profile after an initial release can be controlled to be linear, logarithmic, or some more complex shape, depending upon the composition of the coated layers of the coating and bioactive agent(s) in the coating. In some embodiments, additives can be included in the biodegradable composition to further control the release rate. In preferred aspects, the inventive biodegradable compositions maintain bioactive agent levels within a therapeutic and/or prophylactic range and ideally a relatively constant level for sustained time periods.

In use, a biodegradable implantable medical device (optionally including bioactive agent in the core and/or in a coating on a surface of the core) is positioned within the body at a treatment site. In one such application, an implant is placed into an eye and is left in position, and the biodegradable polymer is allowed to degrade. Upon placement of the implant, and thus exposure of the biodegradable polymer to physiological fluids, bioactive agent is released from the implant. Bioactive agent release can be attributed to diffusion of the bioactive agent through the polymer matrix, and/or degradation of the polymer matrix. Typically, an initial release of the bioactive agent is observed, and over time a sustained release of the bioactive agent is observed. As the biodegradable polymer degrades, bioactive agent continues to be released in a controlled manner, thereby providing a therapeutically effective amount of the bioactive agent over a treatment course to the treatment site. In some aspects, when the implant includes a core, the core of the device can degrade as well, typically after completion of the desired treatment. Some aspects of the invention thus provide a completely degradable device.

These and other aspects and advantages will now be described in more detail.

### BRIEF DESCRIPTION OF THE DRAWING

For a fuller understanding of the nature and desired objects of the present invention, reference is made to the following detailed description taken in conjunction with the accompanying drawing figures wherein like reference characters denote corresponding parts throughout the several views and wherein:

FIG. 1 shows an illustration of a subretinal implant in accordance with one embodiment of the present invention.

FIG. 2 shows a longitudinal cross-sectional view of the subretinal implant of FIG. 1.

FIG. 3 shows an illustration of a side view of a subretinal implant in accordance with one embodiment of the present invention.

FIG. 4 shows a longitudinal cross-sectional view of a subretinal implant in accordance with one embodiment of the invention.

FIG. 5 shows an illustration of a side view of the subretinal implant of FIG. 4.

FIG. 6 shows a perspective view of an implantable device configured for intraocular placement according to one embodiment of the invention.

FIG. 7 shows a view from the bottom of the embodiment illustrated in FIG. 6.

FIG. 8 shows a perspective view of an implantable device configured for intraocular placement according to one embodiment of the invention.

FIG. 9 shows a view from the bottom of the embodiment illustrated in FIG. 8.

FIG. 10 shows transcleral placement of an implantable device according to one embodiment of the invention.

FIG. 11 is a schematic diagram of a spray stream that passes through a focal point.

FIG. 12 is a schematic diagram of a spray stream that expands continuously as it moves away from the spray head.

FIG. 13 is a schematic view of a grid-like coating pattern useful in coating implants of the invention.

FIG. 14 is a schematic view of a grid-like coating pattern superimposed over a core material.

FIG. 15 is a schematic view of a series of first transverse sweeps superimposed over a core material.

FIG. 16 is a schematic depiction of the filament preparation process in accordance with one embodiment of the present invention.

FIG. 17 shows fundus photography of an implanted polycaprolactone/triamcinolone acetonide (PCL/TA) filament in accordance with one embodiment of the invention, at 4 weeks after implant.

FIG. 18 shows fluorescein angiography of an implanted PCL/TA filament in accordance with one embodiment of the invention, at 4 weeks after implant.

FIG. 19 shows optical coherence tomography of the retinal thickness surrounding the implant site for two polycaprolactone (PCL) filaments in accordance with embodiments of the invention, at 4 weeks after implant. Retinal surface (in μm) is represented on the X-axis, and retinal thickness (in μm) is represented on the Y-axis.

FIG. 20 shows in vitro cumulative elution data for a 70:30 PCL/TA implant in accordance with one embodiment of the invention. In the graph, time (in days) is represented on the X-axis, while concentration of triamcinolone (TA, in μg) is represented on the Y-axis.

FIG. 21 shows in vitro cumulative elution data for a 60:40 PCL/TA implant in accordance with one embodiment of the invention. In the graph, time (in days) is represented on the X-axis, while concentration of triamcinolone (TA, in μg) is represented on the Y-axis.

FIG. 22 shows in vitro cumulative elution data for a 50:50 PCL/TA implant in accordance with one embodiment of the invention. In the graph, time (in days) is represented on the X-axis, while concentration of triamcinolone (TA, in μg) is represented on the Y-axis.

FIG. 23 shows optical image and magnification of a subretinal PCL/TA implant in accordance with an embodiment of the invention, following 4 weeks implantation, where A) shows the optic nerve location, B) marks the implant location, C and D) shows the site of the retinotomy, E) is the outer sclera surface, and F) outlines the region of damage to the proximal end of the filament during micro forceps insertion.

FIG. 24 shows histology (H&E staining) of a 150 μm PCL subretinal implant (no drug) in accordance with one embodiment of the invention, following 4 weeks implantation, where A) marks the device location, B) shows the RPE, C) shows the nerve fiber layer, D) shows the choroid and E) shows the sclera.

FIG. 25 shows histology (H&E staining) of a 150 μm PCL/TA subretinal implant in accordance with one embodiment of the invention, following 4 weeks implantation, where A) marks the device location, B) shows the RPE, C) shows the nerve fiber layer, D) shows the choroid, E) shows the sclera and F) identifies the region of vacuolated spaces.

FIG. 26 shows an explanted PCL/TA subretinal implant in accordance with an embodiment of the invention.

FIG. 27 shows in vivo detection of triamcinolone acetonide (TA) following a 4-week subretinal implantation (PCL/TA 60:40) in accordance with one embodiment of the invention.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The invention is directed to implantable medical devices fabricated from a biodegradable material. At least a portion of the device is biodegradable, and this portion is broken down gradually by the body after implantation.

The inventive devices and methods provide improved biodegradable devices that exhibit controlled release of one or more bioactive agents. The term “biodegradable” and is art-recognized and includes polymers, compositions and formulations, such as those described herein, that degrade during use. Such use includes in vivo use (such as in vivo therapy) and in vitro use. In general, degradation attributable to biodegradability involves the degradation of a biodegradable polymer into its component subunits, or digestion (for example, by a biochemical process), of the polymer into smaller, non-polymeric subunits. In certain embodiments, biodegradation may occur by enzymatic mediation, degradation in the presence of water and/or other chemical species in the body, or both.

The present invention is directed to methods and apparatuses for effectively treating a treatment site within a patient's body, and in particular for delivering bioactive agent to limited access regions of the body, such as the eye, ear, sinuses, central nervous system (spinal cord, brain), joints, and the like. According to some embodiments of the invention, degradable implants are provided that can provide treatment to a site within the body for a desired period of time, after which at least a portion of the implant degrades. Such methods and apparatuses in accordance with the present invention can advantageously be used to provide flexibility in treatment duration and type of bioactive agent delivered to the treatment site. In some particular aspects, the present invention has been developed for controllably providing one or more bioactive agents to a treatment site within the body for a desired treatment course.

The term “implantation site” refers to the site within a patient's body at which the implantable device is placed according to the invention. In turn, a “treatment site” includes the implantation site as well as the area of the body that is to receive treatment directly or indirectly from a device component. For example, when the device includes one or more bioactive agents, bioactive agent can migrate from the implantation site to areas surrounding the device itself, thereby treating a larger area than simply the implantation site.

In some aspects, the inventive systems and methods provide biodegradable polymer systems for sustained, site-specific delivery of bioactive agent to limited access regions of the body, such as the eye.

The inventive devices and methods have particular application in the field of ophthalmology. However, one of skill in the art will readily appreciate, upon review of the present description, that inventive devices and methods have utility in other areas of the body as well. Other non-ocular applications include otological (ear), central nervous system (spinal cord, brain), joints, sinuses, and the like.

To facilitate the discussion of the invention, use of the invention to treat ocular sites will be addressed. Ocular treatment is selected because the features of the invention, particularly relating to degradative properties and drug delivery capabilities can be clearly presented. Further, the ability to provide a temporary medical device that can provide superior qualities while reducing risks to the patient can be a significant advance in the field. For purposes of discussion, treatment of ocular sites with particular device configurations is described; however, other device configurations (implant configurations) can utilize the inventive concepts described herein. In some aspects, the polymer systems can be used to deliver bioactive agent to a subretinal area of the eye. In some aspects, the polymer systems can be used to deliver bioactive agent to the vitreous of the eye.

The inventive medical devices and systems are particularly useful for those devices that will come in contact with aqueous systems, such as bodily fluids. Such devices are adapted to release bioactive agent in a prolonged and controlled manner, generally beginning with the initial contact between the device surface and its aqueous environment. It is important to note that the local delivery of combinations of bioactive agents may be utilized to treat a wide variety of conditions utilizing any number of medical devices.

The present invention provides bioactive agent delivery systems for providing sustained delivery of one or more bioactive agents within a selected site of a mammal, and methods for administering or delivering the bioactive agents within the targeted site of a mammal using such delivery systems. The present invention also provides methods for fabricating the delivery systems, in particular, methods for fabricating implants that are used to deliver the one or more bioactive agents. The drug delivery systems and methods overcome limitations of current devices and treatment methods for disease, such as ocular diseases.

In embodiments of the invention, the bioactive agent delivery system comprises one or more implants that can be placed within the eye at a desired treatment site. In particular, the implant comprises a biodegradable polymer matrix including one or more bioactive agents.

In some embodiments, a polymer matrix containing one or more bioactive agents alone forms the implant. It is to be understood that this means that the polymer matrix with the one or more bioactive agents form substantially all of the implant, but that other small amounts of materials may also be contained in the implant due to processing and stabilizing techniques used in forming the implant.

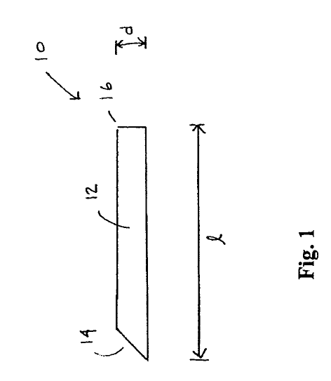

In some aspects, the implant can be utilized for subretinal application. Referring to FIG. 1, implant 10 comprises polymer matrix 12 containing one or more bioactive agents. Implant 10 has length “l” and diameter “d” as shown in FIG. 1. Implant 10 has distal end 14 and proximal end 16. The distal and/or proximal ends of the implant may be tapered, rounded, beveled, blunt, or may have other desirable end shapes. In the embodiment of FIG. 1, implant 10 has a beveled distal end 14 and has a blunt proximal end 16. Features of the polymer matrix will now be described. Illustrative subretinal implants, systems, and methods are described in U.S. Patent Publication No. 2002/0198511 A1 (“Method and Device for Subretinal Drug Delivery,” Varner et al.), and PCT Publication No. WO 2004/028477 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); and related applications.

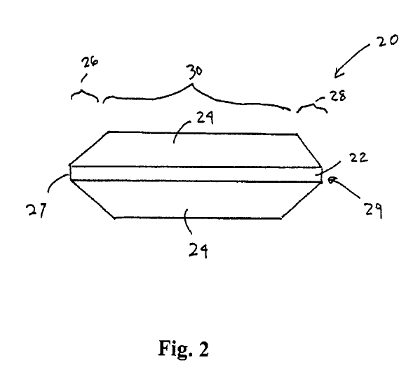

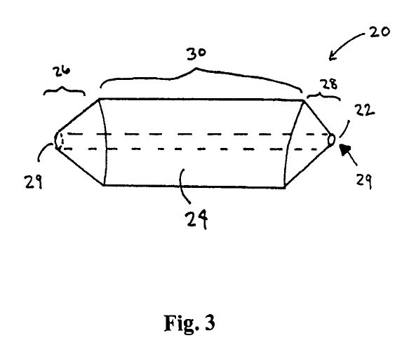

When used for subretinal delivery, the implant can be of any geometric shape and size that can be readily inserted into the eye. Further, once inserted, the implant should not be sized and/or shaped so as to interfere with the functions of the eye (such as vision) and should not cause unnecessary discomfort or damage to the eye. In some embodiments, the implant is rod-like or filament-like in shape. However, the geometry of the device is not limited to filament or rod shapes but, rather, it may also be provided in any other shape suitable for insertion into the eye (e.g., curved or C-shaped devices, coils, thin films, ribbons, blocks, foldable discs, pellets, etc.). In some embodiments, the implant is designed so as to facilitate insertion within the eye. For example, the distal end of the implant may be beveled, tapered, or sharpened so as to facilitate eye entry and/or penetration. Alternatively, the distal end may be blunt or rounded and the device may be inserted through an incision in the eye. While providing an implant with a sharpened distal end may facilitate penetration and entry into the eye, it can potentially make it more challenging for the user to position the implant and may contribute to the implant crossing multiple retinal tissue layers rather than conforming itself into the subretinal space at its final resting position (see, for example, FIGS. 2-3). However, these potential results can be overcome by the use of implantation techniques wherein these factors are taken into account, or by providing an implant with a blunt or rounded distal end. The implant is designed to provide sustained delivery of bioactive agent(s) without major trauma or the need for fluid dissection of the retina.

In some embodiments, the outer diameter of the implant is no greater than about 1000 μm to minimize the incidence of retinal detachments and hemorrhaging. In other embodiments, the outer diameter of the implant is 900 μm or less, in other embodiments 800 μm or less, in other embodiments 700 μm or less, in other embodiments 600 μm or less, in other embodiments 500 μm or less, in other embodiments 400 μm or less, in other embodiments 300 μm or less, in other embodiments 200 μm or less. In some embodiments the diameter of the implant ranges from about 200 μm to about 500 μm.

In some embodiments, the length of the implant is no greater than about 6 mm, in other embodiments no greater than 5 mm, in other embodiments no greater than 4.5 mm, in other embodiments no greater than 4.0 mm, in other embodiments no greater than 3.5 mm. In a specific embodiment the implant is no greater than about 3 mm in length as such lengths have been found to provide the additional benefit of coming to a final resting point within the eye that does not cross multiple tissue layers. However, it is possible to provide implants longer than 3 mm that can be inserted with special care so as to minimize the incidence of multiple tissue layer crossing. The flexibility of the implant can allow it to conform to its final implanted resting position. In yet further embodiments, the length of the implant is 2.9 mm or less, in other embodiments 2.8 mm or less, in other embodiments 2.7 mm or less, in other embodiments 2.6 mm or less, in other embodiments 2.5 mm or less, in other embodiments 2.4 mm or less, in other embodiments 2.3 mm or less, in other embodiments 2.2 mm or less, in other embodiments 2.1 mm or less, and in other embodiments 2.0 mm or less. In some embodiments, the length of the implant ranges from about 2.00 mm to about 3.00 mm.

As the implant becomes smaller in diameter, the insertion and handling of the device becomes more difficult and the amount of bioactive agent(s) that can be contained in the implant is limited. Such factors are taken into account in determining the size of the implant. In some embodiments, the implant is sufficiently rigid to be grasped by a microsurgical instrument that can direct it into the subretinal space and, thus, the implant is designed accordingly. Similarly, as the implant becomes smaller in length, the insertion and handling of the device becomes more difficult and the amount of bioactive agent(s) that can be contained in the implant is reduced. Thus, these factors are taken into account in determining the size of the implant. In one embodiment, the cross-sectional surface area of the device is preferably up to 196250 μm².

Referring to FIGS. 2 and 3, one embodiment of an implant of the type that has a core is shown. The implant configuration can be particularly useful for subretinal placement and treatment. Implant 20 includes core 22, having proximal end 27 and distal end 29, and coating layer 24 comprising polymer matrix-bioactive material. In the embodiment of FIGS. 2 and 3, the coating layer 24 of polymer matrix-bioactive material is coated over the entire length of core 22. The coating layer 24 of polymer matrix-bioactive material includes proximal transition segment 26, distal transition segment 28, and center portion 30. In this embodiment, proximal transition segment 26 and distal transition segment 28 have been feathered (for example, a sloped transition segment).

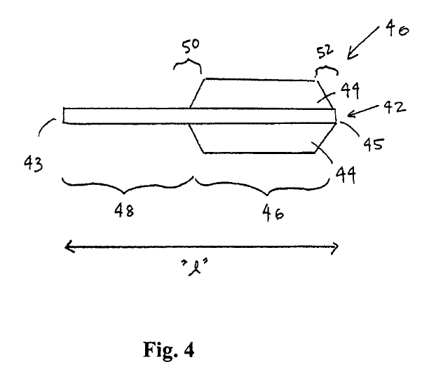

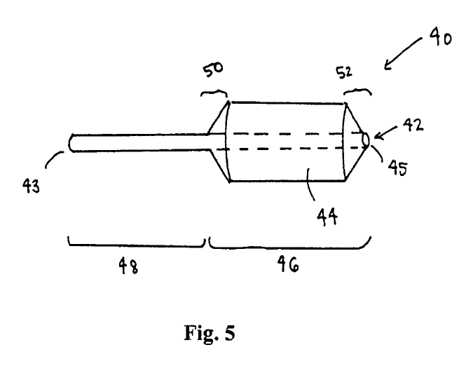

In another embodiment, as shown in FIGS. 4 and 5, implant 40 includes core 42, having proximal end 43 and distal end 45. A coating layer 44 of polymer matrix-bioactive material is coated over a portion of the length “l” of core 42, resulting in coated portion 46 and uncoated portion 48. The uncoated portion 48 may be useful to provide a handling portion by which the implant may be grasped or docked with a surgical instrument (for example, by microsurgical instruments) to prevent any potential damage to the polymer matrix-bioactive material 44 upon handling. In one embodiment, the uncoated portion of the implant device could be left periretinal for easy retrieval in follow-up surgery. In the embodiment of FIGS. 4 and 5, proximal transition segment 50 and distal transition segment 52 of coated portion 46 have been feathered (a sloped transition segment). Without being bound by theory, it is believed that feathering the distal and proximal ends of the implant may enhance the uniformity, processing reproducibility, and ease of implantation.

The cross-sectional shape of the core may be any desired shape, but is typically circular. The diameter of the core is typically less than about 200 μm, in some embodiments ranging from about 10 μm to about 200 μm. In some embodiments, the core comprises titanium-nickel wire. In one embodiment, the core is titanium-nickel wire having a diameter of 80 μm (or the smallest commercially available diameter), thereby maximizing the volume of bioactive agent that can be loaded, while still providing a structure for the coating material.

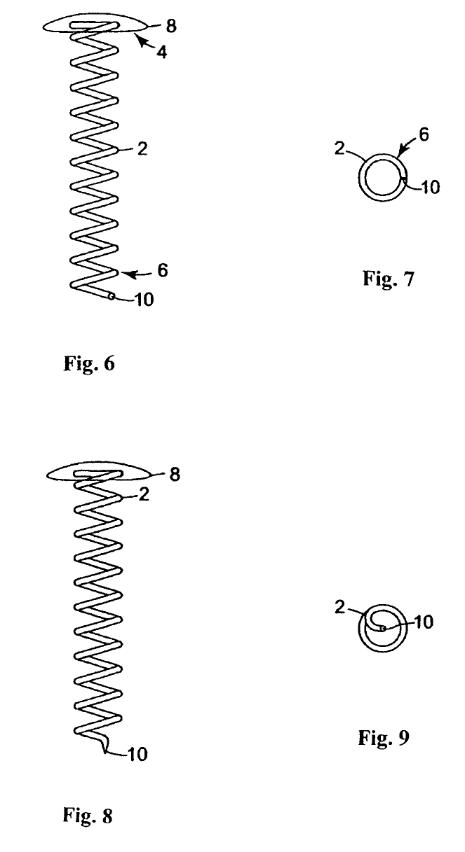

In some aspects, the implant can be utilized for intraocular application. Referring to FIGS. 6-10, an implant according to another embodiment is illustrated. Generally speaking, the implant illustrated in FIGS. 6-10 provides a controlled release bioactive agent delivery device comprising a body member having a direction of extension, a longitudinal axis along the direction of extension, and a proximal end and a distal end, wherein at least a portion of the body member deviates from the direction of extension, the body member including a biodegradable polymer matrix comprising a bioactive agent. As shown in FIG. 6, an implant includes a body member 2 having a proximal end 4 and a distal end 6. FIG. 6 illustrates the body member in a coil configuration. Illustrative implants, systems, and methods for intraocular application are described in U.S. Pat. No. 6,719,750 B2 (“Devices for Intraocular Drug Delivery,” Varner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and related applications.

The distal end 6 of the body member 2 can be positioned at any desirable location relative to the longitudinal axis of the body member. As shown in FIGS. 6 and 7, the distal end 6 of the body member according to one embodiment of the invention can include a tip 10 that is spaced from the longitudinal axis. This configuration is similar to a standard “cork screw” type configuration. In use, the device is inserted through the incision site and then twisted until the controlled delivery device is properly positioned at the treatment site.

Another embodiment is shown in FIGS. 8 and 9, wherein the distal end 6 of the body member includes tip 10 that is positioned at the longitudinal axis of the body member 2. In some embodiments, placement of the tip 10 of the body member 2 at the longitudinal axis can provide advantages, such as ease of insertion of the device at the distal end. It will be readily apparent that various other configurations of the distal end of the body member can be provided, depending upon the desired application.

Further, the proximal end 4 of the body member 2 can also be positioned at any desirable location relative to the longitudinal axis of the body member. FIGS. 6 and 8 illustrate the proximal end 4 of the body member as spaced from the longitudinal axis. However, the proximal end 4 of the body member can be provided at the longitudinal axis as well (not shown in the figures). In some embodiments, placement of the proximal end 4 of the body member 2 at the longitudinal axis can provide advantages, such as ease of fabrication of the device, increased mechanical strength, improved translation of force (since a uniform force can be applied and translated to the body member, with less risk of bending or other deformation of the body member), and the like.

According to the intraocular embodiments of the invention, the coil shape of the body member allows the device to be screwed or twisted into the body through an incision approximately the same size as the outer diameter of the material forming the body member 2. Still further, the coil shape of the body member can act as an anchoring mechanism to maintain the controlled delivery device within the implantation site, and can prevent unwanted movement of the device and unwanted ejection of the device from the implantation site and/or the body. As a result of the coil shape, the controlled delivery device is twisted and unscrewed out of the body during removal of the device.

Generally speaking, the body member of the implantable device is the portion of the controlled release device that is inserted into a patient. The body member can be described as including a proximal end (which is located, upon implantation, towards the exterior of the body), a distal end (which is located, upon implantation, towards the interior of the body), and a longitudinal axis. In use, at least a portion of the body member is inserted into a patient's body. For example, in some embodiments, it can be preferable to position less than 100% of the body member inside the patient's body. The amount of the body member positioned within the body can be determined by the interventionalist, based upon such factors as desired treatment parameters, the particular configuration of the device, the implantation site, and the like.

The body member further includes a direction of extension, and in preferred embodiments, at least a portion of the body member deviates from the direction of extension. In preferred embodiments, the body member includes at least two, three, four, five, six, seven, eight, nine, ten, or more deviations from the direction of extension. In some alternative embodiments, where the body does not include multiple deviations from the direction of extension, the body member can be provided in a “J” or a hook-type configuration.

The deviations from the direction of extension can be provided in any suitable configuration. Exemplary embodiments of such deviations will be described herein for illustrative purposes only, and without intending to be bound by any particular embodiment described herein. The deviations need not be rounded or arcuate. For example, in some embodiments, the body member is provided with a Z-shaped configuration, such that the deviations are angular. Moreover, the deviations need not be in a regular pattern, but can alternatively be provided in a random manner, such that the body member contains random curls or turns. In some embodiments, the deviations are provided in a patterned configuration about the longitudinal axis. Examples of these patterned embodiments include coils, spirals, or patterned Z-shaped turns in the body. Alternatively, the deviations can be provided in a random or non-patterned configuration about the longitudinal axis. According to these particular non-patterned embodiments, the distance of the individual deviations from the longitudinal axis to the outermost periphery of the body member can be selected to provide a desired overall profile of the body member, depending upon the application of the device. For example, it can be desirable, in some applications, to provide an overall profile of the body member having an hourglass shape, alternating ring circumference shapes, and the like.

In some embodiments, the deviations from the direction of extension can be provided in the form of rings. Such individual rings can be concentric (that is, having a common axis, or being coaxial about the longitudinal axis) or eccentric (deviating from a circular path). According to these embodiments, the individual rings are noncontiguous along the body member length, thereby forming individual ribs at positions along the direction of extension of the body member.

Preferred configurations of the body member are coiled or spiral. Generally, in a coil configuration, the individual rings of the coil rotate about the longitudinal axis, and the overall coil is substantially symmetrical about the longitudinal axis. A preferred coil is composed of multiple rings that are substantially similar in circumference along the length, from proximal to distal, of the device. In some preferred embodiments, the rings form a spiral pattern, wherein the circumference of the rings changes over the length of the device. Preferably, the circumference of the rings decreases toward the distal direction of the device, so that the largest ring circumference is located at the proximal region of the device, and the smallest ring circumference is located at the distal region of the device.

Inclusion of deviating portions of the body member provides an increased surface area for delivery of a bioactive agent to an implantation site as compared to a linear device having the same length and/or width. This can provide advantages during use of the device, since this configuration allows a greater surface area to be provided in a smaller length and/or width of the device. For example, in some applications, it can be desirable to limit the length of the device. For example, as will be discussed in more detail herein, it is desirable to limit the length of implants in the eye to prevent the device from entering the central visual field of the eye and to minimize risk of damage to the eye tissues. By providing a body member that has at least a portion of the body member deviating from the direction of extension, the device of the invention has greater surface area (and thus can hold a greater volume of bioactive agent) per length of the device without having to make the cross section of the device, and thus the size of the insertion incision, larger.

Still further, in preferred embodiments, the shape of the body member can provide a built-in anchoring system that reduces unwanted movement of the device and unwanted ejection of the device out of the patient's body, since the shape of the body member requires manipulation to remove it from an incision. For example, for a coil-shaped body member, the device would require twisting, and a Z-shaped body member would require back and forth movement, to remove the device from the implantation site. According to some preferred embodiments, the device does not require additional anchoring mechanisms (such as suturing) to the body tissues, as a result of the self-anchoring characteristics of the device itself. As described in more detail herein, inclusion of a cap 8 on the device can provide further anchoring features of the device.

In some embodiments, when the body member includes two or more deviations from the direction of extension, the spacing of the individual deviations can be selected to provide an optimum combination of such features as increased surface area available for coating, overall dimensions of the device, and the like. For example, when the body member is provided in the form of a coil that includes two or more deviations from the direction of extension, the distance between the individual coils can be selected to be equal to or greater than the diameter of the material forming the body member. In some aspects, if the distance between coils is less than the diameter of the material forming the body member, the amount of surface area available for coating of the body member can decrease, since it can be more difficult to access portions of the surface area of the body member with the coating compositions. In one illustrative embodiment of this aspect of the invention, the body member is formed of a material having a diameter of 0.5 mm, and the distance between each coil of the body member is at least 0.5 mm. These principals can be applied to any configuration of the body member and is not limited to coiled configurations.

The overall dimensions of the implantable device can be selected according to the particular application. For example, the length and/or width of the device can be selected to accommodate the particular implantation site. Some factors that can affect the overall dimensions of the implantable device include the potency of any bioactive agent to be delivered (and thus the volume of bioactive agent required, which impacts the surface area of the device, as discussed herein), the location of the implantation site within the body (for example, how far within the body the implantation site is located), the size of the implantation site (for example, a small area such as the eye or inner ear, or a larger area, such as a joint or organ area), the tissue surrounding the implantation site (for example, vascular tissue or hard, calcinous tissue, such as bone), and the like.

By way of example, when the implantable device is used to deliver bioactive agent(s) to the eye, the device is preferably designed for insertion through a small incision that requires few or no sutures for scleral closure at the conclusion of the surgical procedure. As such, the device is preferably inserted through an incision that is no more than about 1 mm in cross-section, for example, in the range of about 0.25 mm to about 1 mm in diameter, preferably in the range of about 0.25 mm to about 0.5 mm in diameter. As such, the cross-section of the material forming the body member 2 is preferably no more than about 1 mm, for example, in the range of about 0.25 mm to about 1 mm in diameter, preferably in the range of about 0.25 mm to about 0.5 mm in diameter. When the material forming the body member 2 is not cylindrical, the largest dimension of the cross-section can be used to approximate the diameter of the body member for this purpose, for example, when the body member cross-section is square.

When used to deliver bioactive agent(s) to the eye, the body member of the controlled release device preferably has a total length from its proximal end to its distal end that is less than about 1 cm, for example, in the range of about 0.25 cm to about 1 cm. Upon implantation, the body member is positioned within the eye, such that the portion of the controlled delivery device that delivers bioactive agent to the eye chamber is positioned near the posterior segment of the eye. When the controlled delivery device includes a cap 8, the cap is preferably provided with a thickness of less than about 1 mm, more preferably less than about 0.5 mm. According to this particular embodiment, the total length of the controlled delivery device is less than about 1.1 cm, preferably less than about 0.6 cm.

The distal end 6 of the body member can include any suitable configuration, depending upon the application of the device and the site of the body at which the device is to be implanted. For example, in some embodiments, the distal end 6 can be blunt or rounded. In preferred embodiments, the distal end 6 of the body member is configured to pierce the body during implantation of the device into the body. For example, the distal end 6 of the body member can include a sharp or pointed tip. In one preferred embodiment, the distal end 6 of the body member has a ramp-like angle. Preferably, the device according to this embodiment can be utilized to make an incision in the body, rather than requiring separate equipment and/or procedures for making the incision site. If the distal end 6 of the body member 2 is used to pierce the body during insertion, at least the distal end 6 is preferably fabricated of a rigid, non-pliable material suitable for piercing the body. Such materials are well known and can include, for example, polyimide and similar materials. In one such preferred embodiment, the distal end 6 of the body member 2 is utilized to pierce the eye for insertion of the controlled delivery device in the interior of the eye.

In another preferred embodiment, the distal end 6 of the body member 2 can be shaped or bent to form a portion (for example, the distal-most portion of the body member) that is parallel to the longitudinal axis. In one embodiment illustrated in FIGS. 3 and 4, for example, the distal end 6 includes a sharp or pointed tip that is parallel to the longitudinal axis. According to this particular embodiment, the tip located at the distal end 6 of the body member is perpendicular to the plane of incision, thus providing a self-starting tip of the device. While these figures illustrate a sharp tip of the body member, it is understood that any suitable configuration of the distal tip can be provided, utilizing the teaching herein.

The body member 2 can be fabricated from a solid material (a material that does not contain a lumen) or a material containing a lumen, as desired. In the embodiment illustrated in FIGS. 1 to 4, for example, the body member 2 is fabricated from a solid material that is shaped into a coil. Alternatively, the body member 2 can be fabricated from a tubular material that includes a lumen. The choice of a solid or lumen-containing material is not critical to the invention and can be determined based upon availability of materials and processing considerations.

When included, the lumen(s) can extend along the length of the body member 2 or only a portion of the length of the body member 2, as desired. In some embodiments, the lumen(s) can serve as a delivery mechanism for delivery of a desired substance to the implantation site. The substance delivered via the lumen can comprise any of the bioactive agents described herein. The substance delivered via the lumen can be the same or different bioactive agent(s) from that included in the polymer matrix. Further, the substance can be provided in addition to the bioactive agent of the polymer matrix, or in place of the bioactive agent. For example, in one embodiment, one or more substances can be delivered via the lumen, and one or more bioactive agents can be provided to the implantation site from the polymer matrix.

In some embodiments, the lumen can contain a polymer matrix as described herein. According to these particular embodiments, the body member of the device can be provided with or without a coating on its external surface. In some such embodiments, the lumen can be utilized to deliver the bioactive agent(s) to the implantation site. For example, the lumen can contain the polymer matrix, including bioactive agent. According to this particular embodiment, the body member can be provided with a coating on an external surface comprising a suitable polymer only (that is, lacking any bioactive agent). Thus, the bioactive agent is provided to the implantation site in this embodiment principally via the lumen of the body member. In other embodiments, the lumen can include the inventive polymer matrix (including biodegradable polymer and bioactive agent), and the body member is not provided with a coated composition on its external surface.

The lumen can contain any combination of elements, as desired. For example, in some embodiments, the lumen can include only the substance to be delivered. In other embodiments, the lumen can include the substance to be delivered, as well as the polymer matrix. The particular combination of elements to be included in the lumen can be selected depending upon the desired application of the device.

When the lumen is to be provided with a substance and/or polymer matrix, the lumen can be filled with the desired substance and/or polymer matrix prior to inserting the device into the body, or after the device has been inserted into the body. When it is desired to fill the device with the substance after insertion into the body, a port can be provided near the proximal end 4 of the body member 2 for such purpose. The port is in fluid communication with the lumen(s) of the body member and can also be used for refilling the device with the substance and/or polymer matrix before and/or after implantation, when desired.

When the device includes a port, the port is preferably designed such that the needle of an injection mechanism (for example, a syringe) can be inserted into the port and the material to be included in the lumen injected by the injection mechanism. Thus, the material can travel through the port and into the lumen(s) of the body member. The port preferably forms a snug seal about the needle of the injection mechanism to prevent leakage of the material out of the port around the injection mechanism and to provide sterile injection of material into the lumen(s). If desired, fittings or collars (not shown), through which an injection mechanism can be inserted and which form a snug seal about the injection mechanism, can be mounted on the port. Upon injection of the material into the delivery device, the needle of the injection mechanism is removed from the port and the port sealed. Sealing can be accomplished by providing a removable cover (not shown) on the port that can be removed for injection of the substance and replaced when the material has been injected. In a preferred embodiment, the port is fabricated of a self-sealing material through which the injection mechanism can be inserted and which seals off automatically when the injection mechanism is removed. Such materials are known and include, for example, silicone rubber, silicone elastomers, polyolefin, and the like.

In further embodiments, when the device includes more than one lumen, the device can include more than one port. For example, each lumen can be in fluid communication with a plurality of ports. These ports are similar to the single port described above. If desired, the lumens and ports can be arranged such that each lumen can be filled with a different material through a corresponding port (for example, each lumen has its own dedicated port). It can be desirable to include more than one lumen when it is desirable to deliver more than one additional material to the implantation site.

In embodiments where it is desired to deliver one or more additional substances to the implantation site via one or more lumens, the individual lumens can include one or more apertures to allow such delivery. In one embodiment, such apertures are provided at the distal end 6 of the device. In other embodiments, the apertures are provided along the length of the body member 2. The number and size of the apertures can vary depending upon the desired rate of delivery of the substance (when provided) and can be readily determined by one of skill in the art. The apertures are preferably designed such that the substance to be delivered is slowly diffused rather than expelled as a fluid stream from the device. For example, when the device is implanted in the eye, it is preferable to deliver the substance through slow diffusion rather than expulsion of the substance as a fluid stream, which can damage the delicate tissues of the eye. In some embodiments, the polymer matrix in contact with the body can provide a particular porosity to the substance and can assist in controlling the rate of diffusion of the substance from the lumen. When included in the device, the particular location of the apertures can be situated so as to deliver the substance at a particular location once the device is implanted into the body.

In another embodiment, when the body member 2 includes a lumen for delivery of an additional substance to the implantation site, the material forming the body member 2 can be chosen to be permeable (or semi-permeable) to the substance to be delivered from the lumen. According to this particular embodiment, the material can be chosen depending upon the particular application of the device and the substance to be delivered and can be readily determined by one of skill in the art. Examples of suitable permeable materials include polycarbonates, polyolefins, polyurethanes, copolymers of acrylonitrile, copolymers of polyvinyl chloride, polyamides, polysulphones, polystyrenes, polyvinyl fluorides, polyvinyl alcohols, polyvinyl esters, polyvinyl butyrate, polyvinyl acetate, polyvinylidene chlorides, polyvinylidene fluorides, polyimides, polyisoprene, polyisobutylene, polybutadiene, polyethylene, polyethers, polytetrafluoroethylene, polychloroethers, polymethylmethacrylate, polybutylmethacrylate, polyvinyl acetate, nylons, cellulose, gelatin, silicone rubbers, porous fibers, and the like.

According to these particular embodiments, the material used to fabricate the body member 2 can be chosen to provide a particular rate of delivery of the substance, which can be readily determined by one of skill in the art. Further, the rate of delivery of the substance can be controlled by varying the percentage of the body member 2 formed of the permeable (or semi-permeable) material. Thus, for example, to provide a slower rate of delivery, the body member 2 can be fabricated of 50% or less permeable material. Conversely, for a faster rate of delivery, the body member 2 can be fabricated of greater than 50% of permeable material. When one or more portions of the body member 2, rather than the whole body member 2, is fabricated of a permeable or semi-permeable material, the location of the permeable or semi-permeable material can be situated so as to deliver the substance at a particular location once the device is implanted at the implantation site.

In another embodiment, the lumen of the body member 2 can include impermeable dividers located along the length of the lumen. Thus, the lumen of the body member can contain a plurality of compartments, each of which can be filled with a different substance, as desired. These compartments could be filled prior to insertion through an injection port located, for example, in the side of each compartment. In another embodiment, the device can be filled after it is implanted by providing a plurality of conduits, each conduit in fluid communication with a corresponding compartment. These conduits can be provided within the wall of the body member 2, along the circumference of the body member 2. The substances could then be injected through a plurality of ports, each port in fluid communication with a corresponding conduit. Thus, a substance could be injected into the first compartment just below the cap 8 by a port in the center of the cap 8, which delivers the substance directly into the first compartment. A substance injected into the second port, would flow through conduit and would flow through an aperture in the wall of body member 2 into second compartment, and so on. The substance(s) to be delivered can be delivered to the implantation site via any of the methods described herein for the lumen(s).

In another embodiment, each lumen or compartment (as desired) can be designed for selected “opening” or activation by a laser (via heat or photodisruption). For example, a laser could be used to create apertures in the walls of the desired lumen and/or compartment when the particular substance is to be delivered. As such, release of each substance could be controlled upon demand by an interventionalist. Preferably, when a laser is utilized to create such apertures, the wavelength and temperature are controlled to minimize any effects on the polymeric coating composition.

In preferred embodiments, the body member 2 can be fabricated in a way that further increases the surface area of the body member, preferably without increasing the overall dimensions of the device. For example, in one embodiment, the device can be fabricated of multiple strands of material that are entwined or twisted around each other to form the body member 2 (for example, multiple strands of wire can be twisted around each other to form the body member). According to these particular embodiments, any number of individual strands can be utilized to form the body member, for example, 2, 3, 4, or more strands. The number of individual strands twisted to form the body member can be selected depending upon such factors as, for example, the desired diameter of the material forming the body member and/or the overall body member diameter, the desired flexibility or rigidity of the device during insertion and/or implantation, the size of the implantation, the desired incision size, the material used to form the body member, and the like.

As shown in FIG. 6, the body member 2 is preferably cylindrical in shape, with a circular cross-section. However, the cross-sectional shape of the body member 2 is not limited and, for example, can alternatively have square, rectangular, octagonal or other desired cross-sectional shapes.

As shown in FIGS. 6 and 8, a preferred embodiment can include a cap 8 positioned at the proximal end 4 of the body member 2. When included in the device, the cap 8 can assist in stabilizing the device once implanted in the body, thereby providing additional anchoring features of the device. Preferably, the device is inserted into the body through an incision until the cap 8 abuts the incision on the exterior of the body. If desired, the cap 8 can then be sutured to the body at the incision site to further stabilize and prevent the device from moving once it is implanted in its desired location. When the device is implanted in the eye, for example, the device can be inserted into the eye through an incision until the cap 8 abuts the incision. If desired, the cap 8 can then be sutured to the eye, to provide further stabilization as discussed above.

The overall size and shape of the cap 8 is not particularly limited, provided that irritation to the body at the incision site is limited. Preferably, the cap 8 is sized such that it provides a low profile. For example, the dimensions of the cap 8 are preferably selected to provide a small surface area to accomplish such desired features as additional anchoring characteristics of the device, without substantially increasing the overall profile of the device upon implantation. In some embodiments, for example, the cap can be covered by a flap of tissue at the incision site upon implantation, to further reduce potential irritation and/or movement of the device at the implantation and/or incision sites. One illustrative example described in more detail elsewhere herein is the covering of the cap with a scleral cap upon implantation of the device in the eye.

Further, while the cap 8 is illustrated with a circular shape, the cap can be of any shape, for example, circular, rectangular, triangular, square, and the like. In order to minimize irritation to the incision site, the cap preferably has rounded edges. The cap 8 is designed such that it remains outside the implantation site and, as such, the cap 8 is sized so that it will not pass into the implantation site through the incision through which the device is inserted.

As described herein, inclusion of a cap 8 in the device can provide additional anchoring features to the device itself. However, in some embodiments, it can be desirable to further secure the device to provide additional anchoring or securing features at the implantation site. Thus, when desired, the cap 8 can be further designed such that it can be easily sutured or otherwise secured to the surface surrounding the incision and can, for example, contain one or more holes (not shown) through which sutures can pass.

The materials used to fabricate the cap 8 are not particularly limited and include any of the materials previously described for fabrication of the body member 2. Preferably, the materials are insoluble in body fluids and tissues with which the device comes in contact. Further, it is preferred that the cap 8 is fabricated of a material that does not cause irritation to the portion of the body that it contacts (such as the area at and surrounding the incision site). For example, when the device is implanted into the eye, the cap 8 is preferably fabricated from a material that does not cause irritation to the portion of the eye that it contacts. As such, preferred materials for this particular embodiment include, by way of example, various polymers (such as silicone elastomers and rubbers, polyolefins, polyurethanes, acrylates, polycarbonates, polyamides, polyimides, polyesters, polysulfones, and the like), as well as metals (such as those described previously for the body member).

In some embodiments, the cap 8 can be fabricated from the same material as the body member 2. Alternatively, the cap 8 can be fabricated from a material that is different from the body member 2. The cap 8 can be fabricated separately from the body member 2, and subsequently attached to the body member 2, using any suitable attachment mechanism (such as, for example, suitable adhesives or soldering materials). For example, the cap 8 can be fabricated to include an aperture, into which the body member 2 is placed and thereafter soldered, welded, or otherwise attached. In alternative embodiments, the cap 8 and body member 2 are fabricated as a unitary piece, for example, utilizing a mold that includes both components (the body member 2 and cap 8) of the device. The precise method of fabricating the device can be chosen depending upon such factors as availability of materials and equipment for forming the components of the device.

In some aspects, and particularly when the body member is fabricated of a biodegradable material, the cap can be fabricated of a nondegradable material or a material that degrades more slowly than the degradable material forming the body member. This can be desirable, for example, to maintain the features provided by the cap (such as anchoring features) for a period of time at least as long as the time the body member retains some structural integrity at the implantation site. This can reduce risk of a significant intact portion of the body member breaking off the cap and losing an anchoring point at the implantation site.

In some embodiments, the cap 8 can be provided with a polymeric coating. According to these particular embodiments, the polymeric coating provided in connection with the cap 8 can be the same as, or different from, the polymeric coating provided in connection with the body member 2. For example, the particular bioactive agent included in the polymeric coating for the cap 8 can be varied to provide a desired therapeutic effect at the incision site. Exemplary bioactive agents that could be desirable at the incision site include antimicrobial agents, anti-inflammatory agents, and the like, to reduce or otherwise control reaction of the body at the incision site. It will be readily apparent upon review of this disclosure that the first polymer and second polymer can also be selected for the polymeric coating composition provided in connection with the cap 8, to provide a desired polymeric coating specific for the cap, when desired.

In some embodiments, the cap 8 can include a polymeric coating that is the same as the polymer coating provided in connection with the body member 2. According to these embodiments, the polymeric coating can be applied in one step to the entire controlled delivery device (body member and cap), if desired. Alternatively, the polymeric coating can be applied to the cap 8 in a separate step, for example, when the cap 8 is manufactured separately, and subsequently attached to the body member 2.

The inventive implants, systems and methods utilize a polymer matrix that includes biodegradable polymer and one or more bioactive agents. The biodegradable polymer aspects of the invention will now be described in more detail.

Polymer Matrix

As used herein, the term “aliphatic” refers to a linear, branched, or cyclic alkane, alkene, or alkyne. Preferred aliphatic groups in polymeric materials that include phosphoester linkages are linear or branched alkanes having 1 to 10 carbon atoms, or linear alkane groups having 1 to 7 carbon atoms.

As used herein, the term “aromatic” refers to an unsaturated cyclic carbon-containing compound with 4n+2π electrons.

As used herein, the term “heterocyclic” refers to a saturated or unsaturated ring compound having one or more atoms other than carbon in the ring, for example, nitrogen, oxygen or sulfur.

Polymers useful in the polymer matrix of an implant are biocompatible and biodegradable. Representative examples of biodegradable polymers that could be used in forming the polymer matrix of an implant include poly(L-lactic acid), polycaprolactone, poly(lactide-co-glycolide), poly(hydroxybutyrate), poly(hydroxybutyrate-co valerate), polydioxanone, polyorthoesters, polyanhydrides, poly(glycolic acid), poly(D,L lactic acid), poly(glycolic acid-co-trimethylene carbonate), poly(phosphate esters), polyphosphoester urethanes, poly(amino acids), cyanoacrylates, poly(trimethylene carbonates), biodegradable polycarbonates, poly(iminocarbonates), polyesters, copoly(ether-esters), polyalkylene oxalates, polyphosphazenes and copolymers and blends of the above polymers. Biodegradable materials such as cellulose, dextrans, polysaccharides, and hyaluronic acid could also be used.

Selection of the polymers for the polymer matrix can depend, for example, on the desired properties of the implant including the desired bioactive agent(s) that is to be delivered by the implant, and the rate and duration of desired bioactive agent release.

In some embodiments, the biodegradable polymer is made up, in whole or in part, of repeating caprolactone monomer units (e.g., poly(caprolactone) or co-polymers thereof). It has been found that polycaprolactone is well tolerated by the retinal tissue and can elute bioactive agents without eliciting inflammatory response or complications. For example, polycaprolactone can elute steroid for a period of at least 4 weeks without eliciting inflammatory response or complications. Thus, in one embodiment, the implant is formed using a biodegradable polycaprolactone polymer matrix. In one embodiment, the implant is rod-shaped and includes corticosteroid triamcinolone acetonide in a biodegradable polycaprolactone polymer matrix. Such embodiments may include a core or no core.

In some aspects, the biodegradable polymer for the polymer matrix comprises one or more particular degradable polymers containing ester linkages (polyetherester copolymers, terephthalate esters with phosphorus-containing linkages, and segmented copolymers with differing ester linkages); or polycarbonate-containing random copolymers; or copolymers and/or blends of any of these. Each of these polymeric biodegradable materials will be described in detail.

In some embodiments, the polyetherester copolymers are amphiphilic block copolymers that include hydrophilic (for example, a polyalkylene glycol, such as polyethylene glycol) and hydrophobic blocks (for example, polyethylene terephthalate).

In one embodiment, the polyetherester copolymer comprises a first component that is a polyalkylene glycol, and a second component which is a polyester formed from an alkylene glycol having from 2 to 8 carbon atoms and a dicarboxylic acid. The polyalkylene glycol, in one embodiment, is selected from the group consisting of polyethylene glycol, polypropylene glycol, and polybutylene glycol. In one embodiment, the polyalkylene glycol is polyethylene glycol.

In another embodiment, the polyester is selected from the group consisting of polyethylene terephthalate, polypropylene terephthalate, and polybutylene terephthalate. In a preferred embodiment, the polyester is polybutylene terephthalate.

In a particular embodiment, the copolymer is a polyethylene glycol/polybutylene terephthalate block copolymer.

In another embodiment, the polyester has the following structural formula I: 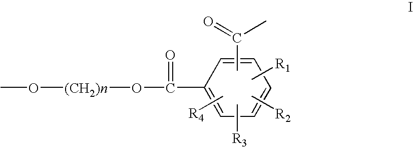
wherein n is from 2 to 8, and each of R₁, R₂, R₃, and R₄ is hydrogen, halogen (such as chlorine, iodine, bromine), nitro-, or alkoxy, and each of R₁, R₂, R₃ and R₄ is the same or different. In one particular embodiment, each of R₁, R₂, R₃ and R₄ are hydrogen. Alternatively, the ester is derived from a binuclear aromatic diacid having the following structural formula II: 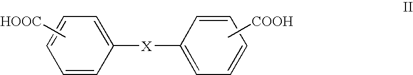
wherein X is —O—, —SO₂—, or —CH₂—.

In one embodiment, the copolymer is a segmented thermoplastic biodegradable polymer comprising a plurality of recurring units of the first component and units of the second component. The first component comprises about 30 weight percent to about 99 weight percent (based upon the weight of the copolymer) of units of the formula III:
—OLO—CO—R—CO— III
wherein L is a divalent organic radical remaining after removal of terminal hydroxyl groups from a poly(oxyalkylene)glycol, O represents oxygen, C represents carbon, and R is a substituted or unsubstituted divalent radical remaining after removal of carboxyl groups from a dicarboxylic acid.

The second component is present in an amount of about 1 weight percent to about 70 weight percent (based upon the weight of the copolymer), and is comprised of units of the formula IV:
—OEO—CO—R—CO— IV
wherein E is an organic radical selected from the group consisting of a substituted or unsubstituted alkylene radical having from 2 to 8 carbon atoms, and a substituted or unsubstituted ether moiety. R is a substituted or unsubstituted divalent aromatic radical.

The poly(oxyalkylene)glycol, in one embodiment, can be selected from the group consisting of poly(oxyethylene)glycol, poly(oxypropylene)glycol, poly(oxybutylene)glycol, and combinations of any one or more of these. In some embodiments, the poly(oxyalkylene)glycol is poly(oxyethylene)glycol.

The poly(oxyethylene)glycol can have a molecular weight in the range of about 200 to about 20,000, or about 200 to about 10,000. The precise molecular weight of the poly(oxyethylene)glycol is dependent upon a variety of factors, including the type of bioactive agent (if any) incorporated into the polymeric matrix.

In one embodiment, E is a radical selected from the group consisting of a substituted or unsubstituted alkylene radical having from 2 to 8 carbon atoms, preferably having from 2 to 4 carbon atoms. Preferably, the second component is selected from the group consisting of polyethylene terephthalate, polypropylene terephthalate, and polybutylene terephthalate. In one embodiment, the second component is polybutylene terephthalate.

In a particular embodiment, the copolymer is a polyethylene glycol/polybutylene terephthalate copolymer.

In one embodiment, the polyethylene glycol/polybutylene terephthalate copolymer can be synthesized from a mixture of dimethylterephthalate, butanediol (in excess), polyethylene glycol, an antioxidant, and catalyst. The mixture is placed in a reaction vessel and heated to about 180° C., and methanol is distilled as transesterification occurs. During the transesterification, the ester bond with methyl is replaced with an ester bond with butylene and/or the polyethylene glycol. In this step, the polyethylene glycol does not react. After transesterification, the temperature is raised slowly to about 245° C., and a vacuum (finally less than 0.1 mbar) is achieved. The excess butanediol is distilled and a prepolymer of butanediol terephthalate condenses with the polyethylene glycol to form a polyethylene glycol/polybutylene terephthalate copolymer. A terephthalate moiety connects the polyethylene glycol units to the polybutylene terephthalate units of the copolymer, and this copolymer is sometimes hereinafter referred to as a polyethylene glycol terephthalate/polybutylene terephthalate copolymer (also referred to as PEGT/PBT or PEG/PBT copolymer). Alternatively, the polyethylene glycol is present as free polyethylene glycol that is mixed with PEGT/PBT copolymer. In another alternative, polyalkylene glycol/polyester copolymers can be prepared as described in U.S. Pat. No. 3,908,201.

The PEGT/PBT copolymer can also be obtained from OctoPlus BV, Bilthoven, The Netherlands, under the product name PolyActive™.

The above discussion of illustrative copolymers is not intended to limit the invention to the specific copolymers discussed, or to any particular synthesis means thereof.

The polymeric matrix can be formulated to provide desired degradation rates. Degradation of the polymeric matrix occurs by hydrolysis of the ester linkages, and/or oxidation of ether groups. Further, when the polymeric matrix includes a bioactive agent, the formulation of the polymeric matrix can be adjusted to control the rate of diffusion of the bioactive agent from the polymer when desired.

In some embodiments, the degradation rate of PEGT/PBT copolymer can be controlled in two general manners. For example, the degradation rate can be increased by including hydrophilic antioxidants in the polymeric material. In addition, or alternatively, the degradation rate can be increased by partially replacing the aromatic groups with aliphatic groups. For example, the more hydrophobic aromatic groups, such as terephthalate groups, can be replaced with less hydrophobic groups, such as diacid groups (for example, succinate). In another example, more hydrophobic butylene groups can be at least partially replaced with less hydrophobic groups, such as dioxyethylene. The degree of replacement can be determined to provide a selected affect on degradation rate.

In accordance with the invention, an increased degradation of the polyetherester copolymer is not accompanied by a significant, deleterious increase in acid formation. Degradation of the copolymer takes place by hydrolysis of ester linkages and oxidation of ether groups, which can generate a certain amount of acid. However, the levels of acid generated during degradation are, in one aspect, lesser than the levels generated by other known degradable polymers (such as PLA), and in another aspect, are not deleterious to tissues and/or bioactive agent. The acidity of the degradation environment can impact the stability of bioactive agents in that environment. Optionally, hydrophilic antioxidants can be included in the polymer material. Such hydrophilic antioxidants will be described in more detail elsewhere herein and can be particularly desirable when the polymeric matrix includes peptide or protein molecules. According to this aspect of the invention, when the protein or peptide molecule is released from the polymeric matrix upon degradation thereof, the protein is not denatured by acid degradation products. This can provide significant advantages over degradable polymers that include polylactic acid (PLA) or copolymers of polylactic acid with glycolic acid (PLGA), where degradation increases acidity of the polymeric environment. These aspects of the invention will be described in more detail with respect to embodiments of the invention where bioactive agents are released from the polymeric matrix.

In some embodiments of the invention, the polymeric material comprises a biodegradable terephthalate copolymer that includes a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, 6,419,709. The respective structures of each of these three classes of compounds, each having a different side chain connected to the phosphorus atom, is as follows:

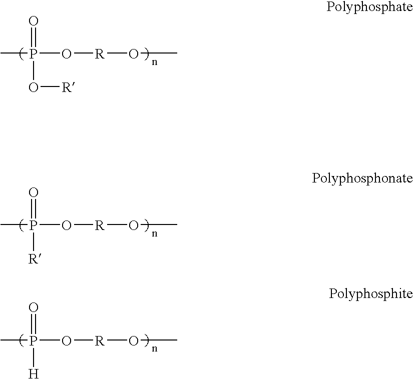

The versatility of these polymers is related to the versatility of the phosphorus atom, which is known for a multiplicity of reactions. Its bonding can involve the 3p orbitals or various 3s-3p hybrids; spd hybrids are also possible because of the accessible d orbitals. Thus, the physicochemical properties of the poly(phosphoesters) can be readily changed by varying either the R or R′ group. The biodegradability of the polymeric material according to these embodiments is related to the physiologically labile phosphoester bond in the backbone of the polymer. By manipulating the backbone or the side chain, a wide range of biodegradation rates are attainable.

An additional feature of the poly(phosphoesters) is the availability of functional side groups. Because phosphorus can be pentavalent, bioactive agents (such as drugs) can be chemically linked to the polymer. For example, bioactive agents with carboxyl groups can be coupled to the phosphorus via an ester bond, which is hydrolyzable. The P—O—C group in the backbone also lowers the glass transition temperature (Tg) of the polymer and, importantly, confers solubility in common organic solvents, which can be desirable for characterization and processing of the polymer.

In one embodiment, the terephthalate polyester includes a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same”). According to this embodiment, the polymeric material comprises recurring monomeric units of the following formula V: 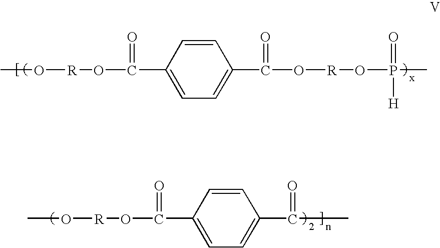
wherein R is a divalent organic moiety. R can be any divalent organic moiety so long as it does not interfere with the polymerization, copolymerization, or biodegradation reactions of the copolymer. Specifically, R can be an aliphatic group, for example, alkylene, such as ethylene, 1,2-dimethylethylene, n-propylene, isopropylene, 2-methylpropylene, 2,2-dimethylpropylene or tert-butylene, tert-pentylene, n-hexylene, n-heptylene, and the like; alkenylene, such as ethenylene, propenylene, dodecenylene, and the like; alkynylene, such as propynylene, hexynylene, octadecynylene, and the like; an aliphatic group substituted with a non-interfering substituent, for example, hydroxy-, halogen-, or nitrogen-substituted aliphatic group; or a cycloaliphatic group such as cyclopentylene, 2-methylcyclopentylene, cyclohexylene, and the like.

R can also be a divalent aromatic group, such as phenylene, benzylene, naphthalene, phenanthrenylene, and the like, or a divalent aromatic group substituted with a non-interfering substituent. Further, R can also be a divalent heterocyclic group, such as pyrrolylene, furanylene, thiophenylene, alkylene-pyrrolylene-alkylene, pyridylene, pyridinylene, pyrimidinylene, and the like; or can be any of these substituted with a non-interfering substituent.

Preferably, however, R is an alkylene group, a cycloaliphatic group, a phenylene group, or a divalent group having the formula VI: 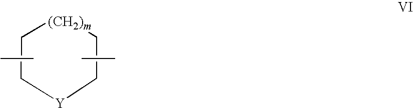
wherein Y is oxygen, nitrogen, or sulfur, and m is 1 to 3. In some preferred embodiments, R is an alkylene group having 1 to 7 carbon atoms and, preferably, R is an ethylene group.

The value of x can vary depending upon the desired solubility of the polymer, the desired Tg, the desired stability of the polymer, the desired stiffness of the final polymers, and the biodegradability and release characteristics desired in the polymer. In general, x is 1 or more, and typically, x varies between 1 and 40. In some embodiments, x is in the range of 1 to 30, or in the range of 1 to 20, or in the range of 2 to 20.

The number n can vary greatly depending upon the biodegradability and the release characteristics desired in the polymer, but typically varies from about 3 to about 7,500, preferably between 5 and 5,000. In some embodiments, n is in the range of about 5 to about 300, or in the range of about 5 to about 200.

The most common general reaction in preparing a poly(phosphite) is a condensation of a diol with a dialkyl or diaryl phosphite according to the following equation:

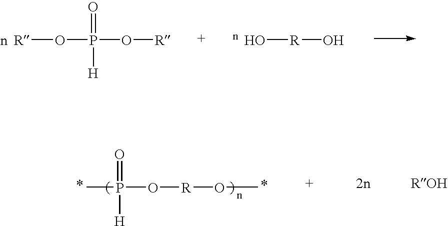

Poly(phosphites) can also be obtained by employing tetraalkyldiamides of phosphorus acid as condensing agents, according to the following equation:

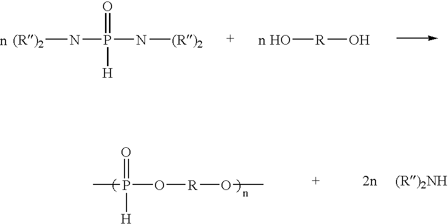

The above polymerization reactions can be either in bulk or solution polymerization. An advantage of bulk polycondensation is that it avoids the use of solvents and large amounts of other additives, thus making purification more straightforward. It can also provide polymers of reasonably high molecular weight.

Typical solvents for solution polycondensation include chlorinated organic solvents, such as chloroform, dichloromethane, or dichloroethane. The solution polymerization is preferably run in the presence of equimolar amounts of the reactants and a stoichiometric amount of an acid acceptor, usually a tertiary amine such as pyridine or triethylamine. The product is then typically isolated from the solution by precipitation with a nonsolvent and purified to remove the hydrochloride salt by conventional techniques known to those of ordinary skill in the art, such as by washing with an aqueous acidic solution, such as dilute hydrochloric acid.

Interfacial polycondensation can be used when high molecular weight polymers are desired at high reaction rates. Mild conditions minimize side reactions. Also, the dependence of high molecular weight on stoichiometric equivalence between diol and phosphite inherent in solution methods is removed. However, hydrolysis of the acid chloride may occur in the alkaline aqueous phase. Phase transfer catalysts, such as crown ethers or tertiary ammonium chloride, can be used to bring the ionized diol to the interface to facilitate the polycondensation reaction. The yield and molecular weight of the resulting polymer after interfacial polycondensation can be affected by reaction time, molar ratio of the monomers, volume ratio of the immiscible solvents, the type of acid acceptor, and the type and concentration of the phase transfer catalyst.

In one embodiment, the process of making the biodegradable terephthalate polymer of formula V comprises the steps of polymerizing p moles of a diol compound having formula VIII: 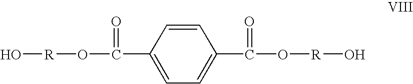
wherein R is as defined above for formula VI, with q moles of dialkyl or diaryl of formula IX: 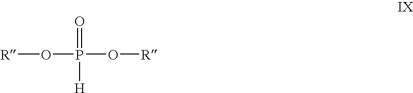
wherein p>q, to form q moles of a homopolymer of formula X, shown below: 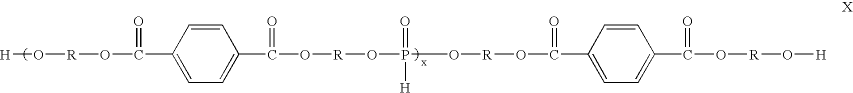
wherein R and x are as defined above for formulae V and VIII. The homopolymer so formed can be isolated, purified and used as is. Alternatively, the homopolymer, isolated or not, can be used to prepare a block copolymer composition of the invention by the steps of: (a) polymerizing as described above, and (b) further reaction the homopolymer of formula X with (p-q) moles of terephthaloyl chloride having the formula XI: 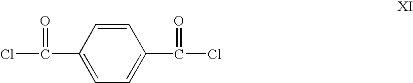
to form the copolymer of formula V.

The polymerization step (a) can take place at widely varying temperatures, depending upon the solvent used, the solubility desired, the molecular weight desired, and the susceptibility of the reactants to form side reactions. Preferably, however, the polymerization step (a) takes place at a temperature in the range of about −40° C. to about 160° C.; for solution polymerization, at a temperature in the range of about 0° C. to about 65° C.; for bulk polymerization, at temperatures of approximately 150° C.

The time required for the polymerization step (a) also can vary widely, depending upon the type of polymerization being used and the molecular weight desired. Preferably, however, the polymerization step (a) takes place in about 30 minutes to about 24 hours.

While the polymerization step (a) can be in bulk, in solution, by interfacial polycondensation, or any other convenient method of polymerization, preferably, the polymerization step (a) is a solution polymerization reaction. Particularly when solution polymerization reaction is used, an acid acceptor is advantageously present during the polymerization step (a). A particularly suitable class of acid acceptor comprises tertiary amines, such as pyridine, trimethylamine, triethylamine, substituted anilines, and substituted aminopyridines. The most preferred acid acceptor is the substituted aminopyridine 4-dimethyl-aminopyridine (“DMAP”).

The purpose of the copolymerization of step (b) is to form a block copolymer comprising (i) the phosphorylated homopolymer chains produced as a result of polymerization step (a), and (ii) interconnecting polyester units. The result is a block copolymer having a microcrystalline structure particularly well-suited to use as a controlled release polymeric matrix.

The copolymerization step (b) of the invention usually takes place at a slightly higher temperature than the temperature of the polymerization step (a), but also can vary widely, depending upon the type of copolymerization reaction used, the presence of one or more catalysts, the molecular weight desired, the solubility desired, and the susceptibility of the reactants to undesirable side reaction. However, when the copolymerization step (b) is carried out as a solution polymerization reaction, it typically takes place at a temperature in the range of about −40° C. to about 100° C. Typical solvents include methylene chloride, chloroform, or any of a wide variety of inert organic solvents.

The time required for the copolymerization of step (b) can also vary widely, depending upon the molecular weight of the material desired and, in general, the need to use more or less rigorous conditions for the reaction to proceed to the desired degree of completion. Typically, however, the copolymerization step (b) takes place during a time of about 30 minutes to about 24 hours.

The terephthalate-poly(phosphite) polymer produced, whether a homopolymer or a block copolymer, is isolated from the reaction mixture by conventional techniques, such as by precipitating out, extraction with an immiscible solvent, evaporation, filtration, crystallization, and the like. Typically, however, the polymer of formula V is both isolated and purified by quenching a solution of the polymer with a non-solvent or a partial solvent, such as diethyl ether or petroleum ether.

In another embodiment, the terephthalate polyester includes a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same”). According to this embodiment, the polymeric material comprises recurring monomeric units shown in Formula XII: 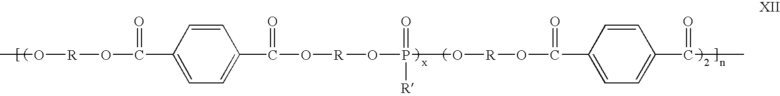
wherein R is a divalent organic moiety as defined above for terephthalate poly(phosphites) of formula V. R′ in the polymeric material of this embodiment is an aliphatic, aromatic, or heterocyclic residue. When R′ is aliphatic, it is preferably alkyl, such as methyl, ethyl, n-propyl, isopropyl, n-butyl, tert-butyl, —C₈H₁₇, and the like; or alkyl substituted with a non-interfering substituent, such as halogen, alkoxy, or nitro.

When R′ is aromatic, it typically contains about 5 to about 14 carbon atoms, or about 5 to about 12 carbon atoms and, optionally, can contain one or more rings that are fused to each other. Examples of suitable aromatic groups include phenyl, naphthyl, anthracenyl, phenanthranyl, and the like.

When R′ is heterocyclic, it typically contains about 5 to 14 ring atoms, preferably about 5 to 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,3-oxathiole, 1,2-pyran, 1,4-pyran, 1,2-pyrone, 1,4-pyrone, 1,2-dioxin, 1,3-dixoin, pyridine, N-alkylpyridinium, pyridazine, pyrimidine, pyrazine, 1,3,5-triazine, 1,2,4-triazine, 1,2,3-triazine, 1,2,4-oxazine, 1,3,2-oxazine, 1,3,5-oxazine, 1,4-oxazine, o-isoxazine, p-isoxazine, 1,2,5-oxathiazine, 1,2,6-oxathiazine, 1,4,2-oxadiazine, 1,3,5,2-oxadiazine, acepine, oxepin, thiepin, 1,2,4-diazepine, indene, isoindene, benzofuran, isobenzofuran, thionaphthene, isothinaphthene, indole, indolenin, 2-isobenzazole, 1,4-pyridein, pyrando-[3,4-b]-pyrrole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquino line, 1,2-benzo-diazine, 1,3-benzodiazine, naphthyridine, pyrido-[3,4-b]pyridine, pyrido[3,2-b]-pyridine, pyrido-[4,3-b]pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In some aspects, when R′ is heterocyclic, it is selected from the group consisting of furan, pyridine, N-alkyl-pyridine, 1,2,3- and 1,2,4-triazoles, indene, anthracene, and purine.

In one embodiment, R′ is an alkyl group or a phenyl group, or an alkyl group having 1 to 7 carbon atoms. In some particular embodiments, R′ is an ethyl group.

The value of x can be varied as described above for polymeric material containing phosphite ester linkages. Similarly, one method for controlling the value of x is to vary the feed ration of the “x” portion relative to the monomer. In this particular embodiment, feed ratios of the ethyl phosphonic dichloride “x” reactant (“EP”) can be used with the terephthaloyl chloride reactant (“TC”) to manufacture the polymer of formula XIII:

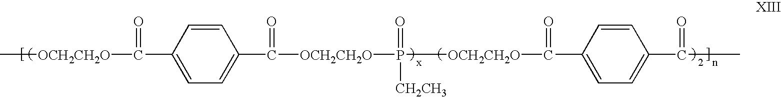

The most common general reaction in preparing a poly(phosphonate) is a dehydrochlorination between a phosphonic dichloride and a diol according to the following equation:

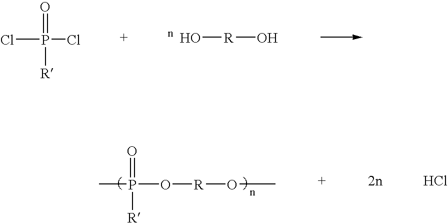

Bulk polycondensation, solution polycondensation, or interfacial polycondensation can be used to synthesize the polymers. A Friedel-Crafts reaction can also be used to synthesize poly(phosphonates). Polymerization typically is effected by reacting either bis(chloromethyl) compounds with aromatic hydrocarbons or chloromethylated diphenyl ether with triaryl phosphonates. Poly(phosphonates) can also be obtained by bulk condensation between phosphorus diimidazolides and aromatic diols, such as resorcinol and quinoline, usually under nitrogen or some other inert gas.

In one embodiment, the process of making the biodegradable terephthalate polymer of formula XIII comprises the steps of polymerizing p moles of a diol compound having formula VIII above, with q moles of a phosphonic dichloride of formula XIV: 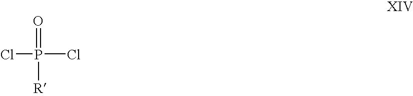
Wherein R′ is defined as above, and p>q, to form q moles of a homopolymer of formula XV shown below: 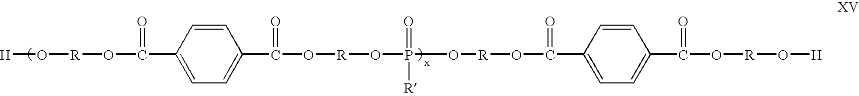
wherein R, R′ and x are as defined above. The homopolymer so formed can be isolated, purified and used as is. Alternatively, the homopolymer, isolated or not, can be used to prepare a block copolymer composition of the invention by: (a) polymerizing as described above, and (b) further reacting the homopolymer of formula XV and excess diol of formula VIII with (p-q) moles of terephthaloyl chloride having the formula XVI: 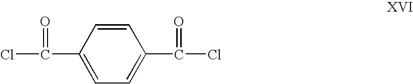
to form the copolymer of formula XII.

The function of the polymerization reaction of step (a) is to phosphorylate the di-ester starting material and then to polymerize it to form the homopolymer. As described above for polymeric material containing phosphite ester linkages, the polymerization step (a) can take place at widely varying temperatures and times.

The addition sequence of the polymerization step (a) can vary significantly depending upon the relative reactivities of the diol of formula VIII, the phosphonic dichloride of formula XIV, and the homopolymer of formula XV; the purity of these reactants; the temperature at which the polymerization reaction is performed; the degree of agitation used in the polymerization reaction; and the like. In some aspects, the diol of formula VIII is combined with a solvent and an acid acceptor, and the phosphonic dichloride is added slowly, for example, a solution of the phosphonic dichloride in a solvent can be trickled in or added dropwise to the chilled reaction mixture of diol, solvent, and acid acceptor, the control the rate of the polymerization reaction.

The purpose and conditions of the copolymerization of step (b) are as described above for polymeric material containing phosphite ester linkages.

The polymer of formula XII, whether a homopolymer or a block polymer, is isolated from the reaction mixture by conventional techniques, such as by precipitating out, extraction with an immiscible solvent, evaporation, filtration, crystallization, and the like. Typically, however, the polymer of formula XII is both isolated and purified by quenching a solution of the polymer with a non-solvent or a partial solvent, such as diethyl ether or petroleum ether.

The polymer of formula XII is usually characterized by a release rate of the bioactive agent in vivo that is controlled at least in part as a function of hydrolysis of the phosphoester bond or the polymer during biodegradation.

Further, the structure of the side chain can influence the release behavior of the polymer. For example, it is expected that conversion of the phosphorus side chain to a more lipophilic, more hydrophobic or bulky group would slow down the degradation process. Thus, for example, release is usually faster from copolymer compositions with a small aliphatic group side chain than with a bulky aromatic side chain.

The lifetime of a biodegradable polymer in vivo also depends upon its molecular weight, crystallinity, biostability, and the degree of crosslinking. In general, the greater the molecular weight, the higher the degree of crystallinity, and the greater the biostability, the slower biodegradation will be. Accordingly, degradation times can vary widely, for example, from less than one day to several months.

In another embodiment, the terephthalate polyester includes a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same”). According to this embodiment, the polymeric material comprises recurring monomeric units shown in Formula XVII: 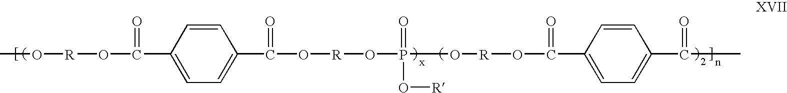
wherein R is a divalent organic moiety as described above for terephthalate poly(phosphites) of Formula V and terephthalate poly(phosphonates) of Formula XII. In some embodiments, R is an alkylene group, a cycloaliphatic group, a phenylene group, or a divalent group of the formula XVIII: 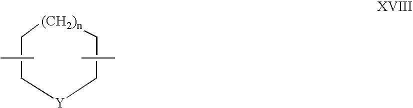
wherein X is oxygen, nitrogen, or sulfur, and n is 1 to 3. In some aspects, R is an alkylene group having 1 to 7 carbon atoms. In some embodiments, R is an ethylene group, a 2-methyl-propylene group, or a 2,2′-dimethylpropylene group. R′ is as describe above for terephthalate poly(phosphites) of Formula V and terephthalate poly(phosphonates) of Formula XII, with the proviso that R′ could also comprise an alkyl conjugated to a biologically active substance to form a pendant bioactive agent delivery system. The value x is 1 or more and can vary as described for terephthalate poly(phosphites) of Formula V and terephthalate poly(phosphonates) of Formula XII. Similarly, one method for controlling the value of x is to vary the feed ratio of the “x” portion relative to the other monomer (for example, varying the feed ratios of the ethyl phosphorodichloridate “x” reactant (“EOP”) relative to the terephthaloyl chloride reactant (“TC”)). The value of n is 0 to 5,000 as described above terephthalate poly(phosphites) of Formula V and terephthalate poly(phosphonates) of Formula XII.

The most common general reaction in preparing poly(phosphates) is a dehydrochlorination between a phosphodichlorinate and a diol according to the following equation:

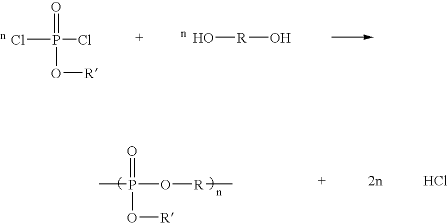

A Friedel-Crafts reaction can also be used to synthesize poly(phosphates). The principals described above for poly(phosphonates) can be utilized for synthesis of poly(phosphates) as well.

The polyphosphates can be synthesized via bulk polycondensation, solution polycondensation, and interfacial polycondensation as described above.

In one embodiment, the process of making a biodegradable terephthalate homopolymer of formula XVII comprises the step of polymerizing p moles of a diol compound having formula XIX: 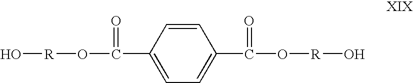
wherein R is as defined above, with q moles of a phosphorodichloridate of formula XX: 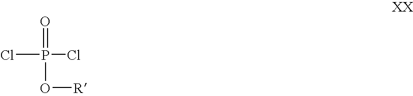
wherein R′ is defined above, and p>q, to form q moles of a homopolymer of formula XXI as shown below: 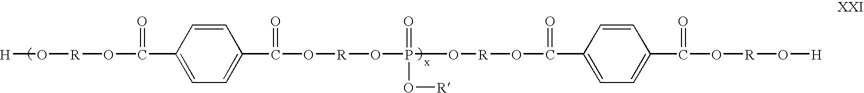
wherein R. R′ and x are as defined above. The homopolymer so formed can be isolated, purified and used as is. Alternatively, the homopolymer, isolated or not, can be used to prepare a block copolymer by (a) polymerizing as described above, and (b) further reacting the homopolymer of formula XXI and excess diol of formula XIX with (p-q) moles of terephthaloyl chloride having the formula XVI to form the polymer of formula XVII.

The function of polymerization steps (a) and (b), as well as conditions therefor are as described above for poly(phosphonates). The addition sequence for the copolymerization step (b) can vary significantly depending upon the relative reactivities of the homopolymer of Formula XXI and the terephthaloyl chloride of Formula XVI; the purity of these reactants; the temperature at which the copolymerization reaction is performed; the degree of agitation used in the copolymerization reaction; and the like. In some aspects, the terephthaloyl chloride of Formula XVI is added slowly to the reaction mixture, rather than vice versa. For example, a solution of the terephthaloyl chloride in a solvent can be trickled in or added dropwise to the chilled or room temperature reaction, to control the rate of the copolymerization reaction.

The polymeric materials comprising a biodegradable terephthalate copolymer that includes a phosphorus-containing linkage (poly(phosphates), poly(phosphonates) and poly(phosphites)) can comprise additional biocompatible monomeric units so long as they do not interfere with the biodegradable characteristics of the polymeric material. Such additional monomeric units can, in some embodiments, offer even greater flexibility in designing the precise release profile desired for targeted bioactive agent delivery or the precise rate of biodegradability desired for structural implants. Examples of such additional biocompatible monomers include, but are not limited to, the recurring units found in polycarbonates, polyorthoesters, polyamides, biodegradable polyurethanes, poly(iminocarbonates), and polyanhydrides.

In some aspects of the invention, the polymeric material of these embodiments is soluble in one or more common organic solvents for ease of fabrication and processing. Common organic solvents can include chloroform, dichloromethane, acetone, ethyl acetate, dimethyl acetamide (DMAC), N-methylpyrrolidone, dimethylformamide, and dimethylsulfoxide. In particular embodiments, the polymeric material is soluble in at least one of these solvents.

The Tg of the polymeric material according to these embodiments can vary widely depending upon the branching of the diols used to prepare the polymer, the relative proportion of phosphorus-containing monomer used to make the polymer, and the like. However, in some aspects, the Tg is within the range of about −10° C. to about 100° C., or in the range of about 0° C. to about 50° C.

When working with poly(phosphates) and poly(phosphonates), the structure of the side chain can influence the release behavior of the polymer. For example, it is generally expected that, with the classes of poly(phosphoesters) described herein, conversion of the phosphorus side chain to a more lipophilic, more hydrophobic or bulky group would slow down the degradation process. For example, release would usually be faster from copolymer compositions with a small aliphatic group side chain than with a bulky aromatic side chain.

The terephthalate poly(phosphites) of formula V are usually characterized by a release rate of the bioactive agent in vivo that is controlled at least in part as a function of hydrolysis of the phosphoester bond of the polymer during biodegradation. However, poly(phosphites) do not have a side chain that can be manipulated to influence the rate of biodegradation.

In the case of biodegradable terephthalate poly(phosphite) polymer in vivo depends sufficiently upon its molecular weight, crystallinity, biostability, and the degree of cross-linking to achieve acceptable degradation rates. In general, the greater the molecular weight, the higher the degree of crystallinity, and the greater the biostability, the slower biodegradation will be.

In still further embodiments of the invention, the polymeric material comprises a copolymer comprising a biodegradable, segmented molecular architecture that includes at least two different ester linkages. According to these particular embodiments, the polymeric material can comprise block copolymers (of the AB or ABA type) or segmented (also known as multiblock or random-block) copolymers of the (AB)ₙ type. These copolymers are formed in a two (or more) stage ring opening copolymerization using two (or more) cyclic ester monomers that form linkages in the copolymer with greatly different susceptibilities to transesterification. These embodiments are described, for example, in U.S. Pat. No. 5,252,701 (Jarrett et al., “Segmented Absorbable Copolymer”) and will now be described in some detail herein.

In one aspect, the polymeric material comprises a copolymer comprising a biodegradable, segmented molecular architecture that includes at least two different ester linkages. Generally speaking, the segmented molecular architecture comprises a plurality of fast transesterifying linkages and a plurality of slow transesterifying linkages. The fast transesterifying linkages have a segment length distribution of greater than 1.3. Sequential addition copolymerization of cyclic ester monomers is utilized in conjunction with a selective transesterification phenomenon to create biodegradable copolymer molecules with specific architectures.

The sequential addition polymerization process of this embodiment is a two (or more) stage ring opening copolymerization using two (or more) cyclic ester monomers that form linkages in the copolymer with greatly different susceptibilities towards transesterification (also referred to herein as “selective transesterification”). For example, such a pair of monomers is ε-caprolactones which forms slow reacting (transesterifying) caproate linkages and glycolide that forms fast reacting glycolate linkages when conventional tin catalysts are employed.

Other parent monomers that can be useful in this process include: p-dioxanone, dioxepanone, deltavalerolactone, beta-butyrolactone, ε-decalactone, 2,5-diketomorpholine, pivalolactone, alpha, alpha-diethylpropiolactone, 6,8-dioxabicyclo octane-7-one, ethylene carbonate, ethylene oxalate, 3-methyl-1,4-dioxane-2,5-dione, 3,3-dimethyl 1,4-dioxane-2,5-dione, substituted glycolides, and substituted lactides. Other cyclic esters described in the art can also be employed with the scope of this invention. These monomers can be categorized as to their susceptibility towards transesterification.

The first stage (Stage I) of the copolymerization consists of a statistical copolymer that has a high content of the slower transesterifying (for example, caproate) linkages and a low content of fast reaction (for example, glycolate) linkages. This prepolymer forms a framework of segments consisting of runs of consecutive caproate linkages with interspersed short glycolate segments. The length and distribution of these segments is affected by such factors as monomer feed composition, the reactivity ratios of the monomers, and the degree of transesterification that occurs in this stage of the reaction. This framework, then, consists of segments with different reactivities for transesterification.

The second stage (Stage II) of the copolymerization consists of the addition of the faster reacting monomer (for example, glycolide) and continuation of the reaction for a specified length of time. The difference in transesterification reactivities of the two segments in the prepolymer preserves the caproate segments in the final copolymer. The second stage initially forms long glycolate segments, most likely at the ends of the Stage I prepolymer. Through transesterification, glycolate linkages from the initially long Stage II glycolate segments are gradually transferred into the shorter glycolate segments in the Stage I prepolymer. The result is a more narrow distribution of glycolate segment lengths. The resulting copolymer has a segmented architecture, which is determined by the Stage I prepolymer framework, the final composition and the difference in transesterification rates. The distribution of segment lengths changes as a function of time after addition of the second stage. This distribution has a marked effect on material properties. In this way, a wide range of material properties can be easily achieved by varying the reaction time for the second and subsequent stages.

This mechanism is not necessarily limited to the caprolactone-glycolide pair. It is known that trimethylene carbonate shows similar behavior to caprolactone when copolymerized with glycolide, and 1-lactide behaves similarly to glycolide when copolymerized with trimethylene carbonate. The observed differences in transesterification rates can be due to the interaction of the linkages with the catalyst. Without intending to be bound by a particular theory, it is believed that linkages within the polymer chain that promote coordination with the catalyst complex would be expected to be more susceptible to undergo transesterification reactions. Such linkages are termed “fast reacting” linkages. It is believed that any combination of a linkage having a fast transesterification rate with a linkage having a slow transesterification rate (or “slow reacting linkage”) can be used to prepare specific architectures in a copolymer of those linkages.

Given the above reasoning, monomers, and the linkages formed from them, can be categorized according to their predicted susceptibilities toward transesterification. The following monomers would be expected to form fast reacting linkages: glycolide, 3-methyl-1,4-dioxane-2,5-dione, 3,3-diethyl-1,4-dioxan-2,5-dione, combinations of any of these, and other substituted “glycolide” type monomers.

The following monomers would be expected to form slow reacting linkages: 1,4-dioxan-2-one (hereafter referred to as “dioxanone linkages”), 1,4-dioxepan-2-one, 1,5-dioxepan-2-one, delta-valerolactone ε-decalactone, pivalolactone, gamma-butyrolactone, ethylene carbonate, trimethylene carbonate, ε-caprolactone, 6,8-dioxabicyclooctane-7-one. Other monomers known to copolymerize should be categorizable according to their reactivities. The reactivities of some of these monomers, however, are difficult to predict. These monomers include: 2,5-diketomorpholine, beta-butyrolactone, propiolactone, and ethylene oxalate. Other cyclic esters described in the art can also be employed with the scope of this invention. The above categorizations are based upon theory, and actual categorization of reactivities can be accomplished experimentally. In some embodiments, the slow transesterifying linkages are selected from trimethylene carbonate, caproate, and dioxanone linkages.

Determination of whether a monomer comprises a fast or slow transesterifying linkage can involve the following test. A copolymer of the monomer of interest and glycolide are prepared using the sequential addition method. The copolymer is made with 100% monomer in the first stage and 100% glycolide (GLY) in the second stage. The following reaction conditions are employed:

Stage I

| | |
|---|---|
| Time | 40 minutes |
| Temperature | 165° C. for 25 minutes, then increased to 180° C. |
| | over 15 minutes |
| Charge | Monomer: 65.10 g |
| | SnCl₂ 2H₂O: 4.09 mg |
| | Diethylene glycol: 7.8 μl |

Stage II

| | |
|---|---|
| Time | 2 hours |
| Temperature | 180° C. to 210° C. over 30 minutes |
| | 210° C. for 1.5 hours |
| Charge | Gly 134.9 g |

The resulting copolymer is ground and placed in vacuum oven at 110° C., <1 mmHg overnight. Thermal analysis and ¹³C NMR analysis are then performed on the sample. If the block length is equal to or greater than 30, the final glycolate weight percent is 68%, and the inherent viscosity is about 1.0 dL/g, then the monomer comprises a slow transesterifying linkage. An inherent viscosity substantially less than about 1.0 dL/g, means that the polymer formed is unstable at the test conditions.

In some embodiments, the copolymer has an inherent viscosity of greater than about 0.1 dL/g (concentration of 0.5 g/dL in a solvent, for example hexafluoroacetone sesquihydrate). For an article of manufacture, such as a surgical suture, requiring an industry acceptable tensile (or other) strength value, an inherent viscosity of about 1.0 dL/g (0.5 dL/g in a solvent) or greater is preferred. For an article of manufacture such as a controlled release device, where a strength value is not required, the copolymer can have an inherent viscosity of lower than about 1.0 dL/g (0.5 g/dL in a solvent).

According to the invention, the copolymer can be manufactured by sequential addition of at least two different cyclic ester monomers in at least two stages. The first cyclic ester monomer is selected from carbonates and lactones, and mixtures thereof. The second cyclic ester monomer is selected from lactides and mixtures thereof. The sequential addition comprises the following steps:
(1) first polymerizing a first stage at least the first cyclic ester monomer in the presence of a catalyst at a temperature in the range of about 160° C. to about 220° C. to obtain a first polymer melt;
(2) adding at least the second cyclic ester monomer to the first polymer melt; and
(3) copolymerizing in a second stage the first polymer melt with at least the second cyclic ester monomer to obtain a second copolymer melt.

The process also comprises transesterifying the second copolymer melt for up to about 5 hours at a temperature of greater than about 180° C.

In one embodiment of the process, the first polymerization step comprises polymerizing in the first stage from about 80 mole % of the first cyclic ester monomer. The remaining mole %, if any, comprises the second cyclic ester monomer. In another embodiment of the process, the first polymerizing step comprises polymerizing in the first stage up to about 90 mole % of the first cyclic ester monomer. In still another embodiment of the process, the step of adding at least the second cyclic ester monomer to the first polymer melt comprises adding more than about 80 mole % of the second cyclic ester monomer. The remaining mole percentage, if any, comprises the first cyclic ester monomer. In a specific embodiment of the process, the step of adding at least the second cyclic ester monomer to the first polymer melt comprises adding 100 mole % of the second cyclic ester monomer.

Another process for manufacturing a copolymer having a biodegradable, segmented molecular architecture comprises sequential addition of at least two different cyclic ester monomers in three stages. The first cyclic ester monomer is selected from carbonates, lactones, and mixtures of carbonates and lactones. The second cyclic ester monomer is selected from lactides and mixtures thereof. The sequential addition comprises the following steps:
(1) first polymerizing in a first stage at least the first cyclic ester monomer in the presence of a catalyst at a temperature in the range of about 160° C. to about 220° C. to obtain a first polymer melt;
(2) first adding at least the second cyclic ester monomer to the first polymer melt;
(3) second copolymerizing in a second stage the first polymer melt with at least the second cyclic ester monomer to obtain a second copolymer melt;
(4) second adding at least the second cyclic ester monomer to the second copolymer melt; and
(5) copolymerizing in a third stage the second copolymer melt with at least the second cyclic ester monomer to obtain a third copolymer melt.

The process also comprises transesterifying the third copolymer melt from up to about 5 hours at a temperature of greater than about 180° C.

In one embodiment of this three-stage process, the first polymerizing step comprises polymerizing in the first stage about 80 mole % or more of the first cyclic ester monomer. The remaining mole percentage, if any, comprises the second cyclic ester monomer. In another embodiment, the first stage comprises polymerizing up to about 90 mole % of the first cyclic ester monomer. In still another embodiment, the addition of the second cyclic ester monomer to the first polymer melt and/or the addition of the second cyclic ester monomer to the second copolymer melt comprise adding more than about 80 mole % of the second cyclic ester monomer. The remaining mole percentage, if any, comprises the first cyclic ester monomer. In a specific embodiment of the process, the addition of the second cyclic ester monomer to the first polymer melt and/or the addition of the second cyclic ester monomer to the second copolymer melt comprises adding 100 mole % of the second cyclic ester monomer.

Optionally, the process can involve polymerization in the presence of a metal coordination catalyst and/or an initiator. In some embodiments, the initiator can be selected from monofunctional and polyfunctional alcohols.

It is understood the catalyst type and level of catalyst employed will affect both the relative polymerization and transesterification rates of the cyclic esters of the invention. By proper choice of both catalyst type and level, copolymers with specific architecture can be prepared in a controllable manner and within a reasonable amount of time. Catalysts such as stannous octoate or stannous chloride dihydrate are preferred; however, other catalysts known in the art to be effective in the ring opening polymerization of cyclic esters are also suitable in accordance with these embodiments of the invention.

The types of architectures that can be made utilizing this process can be AB diblock, ABA triblock, or segmented copolymers with wide or narrow block length distributions. Diblocks and triblocks are made using monofunctional or difunctional initiators (alcohols) in the Stage I reaction and by using only the slow transesterification rate linkage to form a Stage I homopolymer. The Stage II linkages can only transesterify within the Stage II segment, preserving the diblock or triblock architecture.

It is generally preferred to conduct the sequential polymerization in a single reaction vessel, by sequentially adding the monomers thereto. However, if desired, one or more of the stages can be polymerized in separate reaction vessels, finally combining the stages for transesterification in a single reaction vessel. Such a process would allow the use of a cyclic polyester forming monomers for one or more of the stages.

Transesterification in aliphatic polyesters derived from cyclic monomers is known in the art. For example, Gnanou and Rempp, Macromol. Chem., 188:2267-2275 (1987) have described the anion polymerization of ε-caprolactone in the presence of lithium alkoxides as being a living polymerization that is accompanied by simultaneous reshuffling. According to this reference, if reshuffling occurs between two different molecules, it can be referred to as “scrambling.” If reshuffling occurs intramolecularly, it is called “back-biting,” and it results in the formation of cycles, the remaining linear macromolecules are of lower molecular weight, but they still carry an active site at the chain end.

In still further embodiments, the biodegradable polymeric matrix comprises a random copolymer comprising at least one carbonate unit as the major component, the carbonate copolymerized with at least one second monomeric component. According to these embodiments, certain aliphatic carbonates can form highly crystalline random copolymers with other monomer components, so long as the appropriate carbonate is present as the major component. These copolymers can provide one or more advantages, such as relatively high modulus and tensile strength, controllable biodegradation rates, blood compatibility, and biocompatibility with living tissue. In preferred aspects, these copolymers also induce minimal inflammatory tissue reaction, as biodegradation of the carbonate polymer by hydrolytic depolymerization results in degradation substances having physiologically neutral pH. Exemplary random copolymers are described, for example, in U.S. Pat. No. 4,891,263 (Kotliar et al.), U.S. Pat. No. 5,120,802 (Mares et al.), U.S. Pat. No. 4,916,193 (Tang et al.), U.S. Pat. No. 5,066,772 (Tang et al.), and U.S. Pat. No. 5,185,408 (Tang et al.).

According to these embodiments, the copolymers are random copolymers comprising as a minor component one or more recurring monomeric units, and as a major component, a recurring carbonate monomeric unit of the following general structures (XXII): 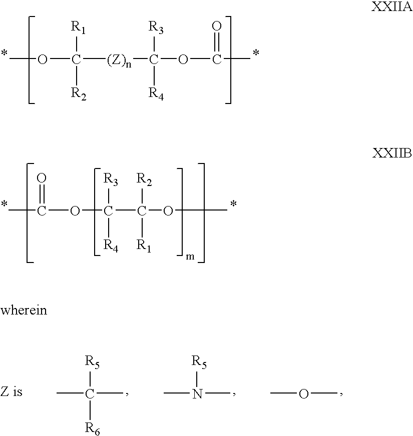
or combinations thereof, where Z is selected such that there are no adjacent heteroatoms;
n and m are the same or different and are integers from about 1 to about 8; and
R₁, R₂, R₃, and R₄ are the same or different at each occurrence and are hydrogen, alkoxyaryl, aryloxyaryl, arylalkyl, alkyarylalkyl, arylalkylaryl, alkylaryl, arylcarbonylalkyl, aryloxyalkyl, alkyl, aryl, alkylcarbonylalkyl, cycloalkyl, arylcarbonylaryl, alkylcarbonylaryl, alkoxyalkyl, or aryl or alkyl substituted with one or more biologically compatible substituents such as alkyl, aryl, alkoxy, aryloxy, dialkyamino, diarylamino, alkylarylamino substituents;
R₅ and R₆ are the same or different and are R₁, R₂, R₃, R₄, dialkylamino, diarylamino, alkylarylamino, alkoxy, aryloxy, alkanoyl, or arylcarbonyl; or any two of R₁ to R₆ together can form an alkylene chain completing a 3, 4, 5, 6, 7, 8, or 9 membered monocyclic, alicyclic, spiro, bicyclic, and/or tricyclic ring system, which system can optionally include one or more non-adjacent carbonyl, oxa, alkylaza, or arylaza groups;
with the proviso that at least one of R₁ to R₆ is other than hydrogen.

Illustrative of useful R₁, R₂, R₃, and R₄ groups are hydrogen; alkyl such as methyl, ethyl, propyl, butyl, pentyl, hexyl, septyl, octyl, nonyl, tert-butyl, neopentyl, isopropyl, sec-butyl, dodecyl, and the like; cycloalkyl such as cyclohexyl, cyclopentyl, cyclooctyl, cycloheptyl, and the like; alkoxyalkyl such as methoxymethylene, ethoxymethylene, butoxymethylene, propoxyethylene, pentoxybutylene, and the like; aryloxyalkyl and aryloxyaryl such as phenoxyphenylene, phenoxymethylene and the like; and various substituted alkyl and aryl groups such as 4-dimethylaminobutyl, and the like.

Illustrative of other R₁ to R₄ groups are divalent aliphatic chains, which can optionally include one or more oxygen, trisubstituted amino or carbonyl groups, such as —(CH₂)₂—, —CH₂(O)CH₂—, —(CH₂)₃—, —CH₂—CH(CH₃)—, —(CH₂)₄—, —(CH₂)₅—, —CH₂OCH₂—, —(CH₂)₂—N(CH₃)CH₂—, —CH₂C(O)CH₂—, —(CH₂)₂—N(CH₃)—(CH₂)₂—, and the like, and divalent chains to form fused, spiro, bicyclic or tricyclic ring systems, such as —CH(CH₂CH₂)₂CH—, —CH(CH₂CH₂CH₂)₂CH—, —CH(CH₂)(CH₂CH₂)CH—, —CH(CH₂)(CH₂—CH₂CH₂)CH—, —CH(C(CH₃)₂)(CH₂CH₂)CH—, and the like.

Illustrative of useful R₅ and R₆ groups are the above-listed representative R₁ to R₄ groups, including OCH₂C(O)CH₂, (CH₂)₂—NCH₃—, —OCH₂C(O)CH₂—, —O—(CH₂)₂—O—, alkoxy such as propoxy, butoxy, methoxy, isopropoxy, pentoxy, nonyloxy, ethoxy, octyloxy, and the like; dialkylamino such as dimethylamino, methylethylamino, diethylamino, dibutylamino, and the like; alkanoyl such as propanoyl, acetyl, hexanoyl, and the like; arylcarbonyl such as phenycarbonyl, p-methylphenyl carbonyl, and the like; and diarylamino and arylalkylamino such as diphenylamino, methylphenylamino, ethylphenylamino, and the like.

Preferred for use in accordance with these embodiment are random copolymers comprising as a major component, carbonate recurring units of the structure illustrated in Formula XXIIA, wherein Z is —(R₅—C—R₆)—, or a combination thereof; n is 1, 2, or 3; and R₁ to R₆ are as defined above, preferably where aliphatic moieties included in R₁ to R₆ include up to about 10 carbon atoms and the aryl moieties include up to about 16 carbon atoms.

Illustrative of these preferred copolymers are those wherein, in the major component, n is 1 and Z is of the formula XXIII: 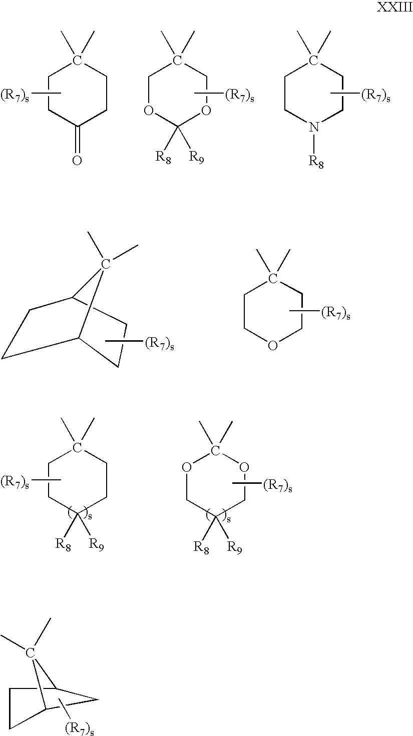
where —C— denotes the center carbon atom of Z, when Z is —C(R₅)(R₆)—; R₇ is the same or different and is aryl, alkyl or an alkylene chain completing a 3 to 16 membered ring structure, including fused, spiro, bicyclic and/or tricyclic structures, and the like; R₈ and R₉ are the same or different at each occurrence and are R₇ or hydrogen, and s is the same or different at each occurrence and is 0 to 3, and the open valencies are substituted with hydrogen atoms.

Also illustrative of these preferred major components are those comprising recurring units of the formula XXIV: 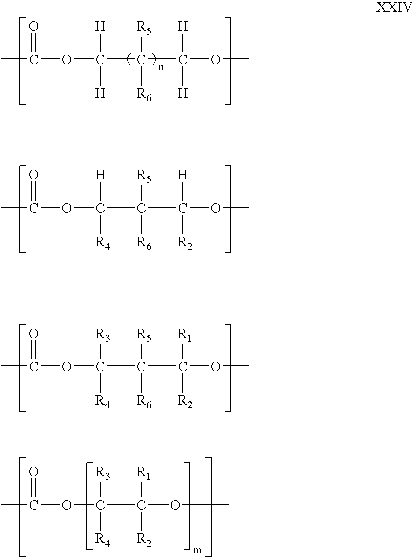
wherein:
R₁, R₂, R₃, and R₄, are the same or different at each occurrence and are hydrogen, alkyl such as methyl, ethyl, n-propyl, isopropyl, n-butyl, sec-butyl, t-butyl, neopentyl, and the like; phenyl; anisyl; phenylalkyl, such as benzyl, phenethyl, and the like; phenyl substituted with one or more alkyl or alkoxy groups such as tolyl, xylyl, p-methoxyphenyl, m-ethoxyphenyl, p-propoxyphenyl, and the like; and alkoxyalkyl such as methoxymethyl, ethoxymethyl, and the like; R₅ and R₆ are the same or different and are R₁ to R₄; alkoxy, alkanoyl, arylcarbonyl, dialkylamino; or any two of R₁ to R₆ together can form alkylene chain completing 4, 5, 6, 7, 8, or 9 membered monocyclic, spiro, bicyclic and/or tricyclic ring structure which structure can optionally include one or more non-adjacent divalent carbonyl, oxa, alkylaza, or arylaza groups with the proviso that at least one of R₁ or R₆ is other than hydrogen; and
n and m are the same or different and are 1, 2, or 3.

Particularly preferred for use in these embodiments are random copolymers comprising as a major component, recurring units of the formula XXV: 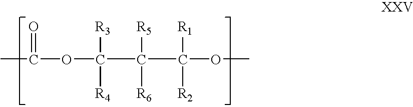
wherein:
R₁ to R₄ are the same or different and are alkyl, hydrogen, alkoxyalkyl, phenylalkyl, alkoxyphenyl, or alkylphenyl, wherein the aliphatic moieties include 1 to 9 carbon atoms; and
R₅ and R₆ are the same or different at each occurrence and are selected from the group of R₁ to R₄ substituents, aryloxy, and alkoxy, or R₅ and R₆ together can form an aliphatic chain completing a 3 to 1 membered spiro, bicyclic, and/or tricyclic structure which can include one or two non-adjacent oxa, alkylaza, or arylaza groups, with the proviso that at least one of R₁ to R₄ is other than hydrogen.

Preferably, the random copolymer comprises as a major component, recurring monomeric units of the following formula XXVI: 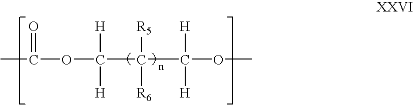
wherein:
n is 1;
R₅ and R₆ are the same or different and are hydrogen, phenyl, phenylalkyl, or phenyl or phenylalkyl substituted with one or more alkyl or alkoxy groups; or alkyl or R₅ and R₆ together make a divalent chain forming a 3 to 6 membered spiro, bicyclic, and/or tricyclic ring structure which can include one or two non-adjacent carbonyl, oxa, alkylaza, or arylaza groups, with the proviso that at least one of R₅ and R₆ is other than hydrogen.

In some aspects of the invention, the random copolymer comprises as a major component, recurring monomeric units of Formula XXVI, particularly when R₅ and R₆ are the same or different and are alkyl, phenyl, phenylalkyl, or phenyl or phenylalkyl substituted with one or more alkyl or alkoxy groups; or a divalent chain forming a 3 to 10 membered, preferably to 7 membered, spiro or bicyclic ring structure that can optionally include one or two non-adjacent oxa, carbonyl, or disubstituted amino groups. It can be particularly preferred that R₅ and R₆ are the same or different and are phenyl, alkylphenyl or phenylalkyl such as tolyl, phenethyl or phenyl, or lower alkyl of 1 to 7 carbon atoms such as methyl, ethyl, propyl, isopropyl, n-butyl, tertiary butyl, pentyl, neopentyl, hexyl, and secondary butyl.

In particular embodiments utilizing Formula XXVI, R₅ and R₆ are the same or different, and are lower alkyl having about 1 to about 4 carbon atoms, and do not differ from each other by more than about 3 carbon atoms, and preferably by not more than about 2 carbon atoms. In some aspects, R₅ and R₆ are the same and comprise alkyl of about 1 to 2 carbon atoms, and in some aspects, methyl for each of R₅ and R₆.

According to these embodiments, the copolymers include a minor component comprising one or more other recurring monomer units. The minor component of the random copolymers of the invention can vary widely.

Illustrative of the second recurring monomeric components are those derived from carbonates, including but not limited to certain of the monomeric units included within the scope of Formula XXIIA wherein n is 0 to 8 within (Z)ₙ, and Formula XXIIB and Formula XXVI, wherein n=1, particularly those less preferred as the major component, and those derived from substituted or nonsubstituted ethylene carbonates, tetramethylene carbonates, trimethylene carbonates, pentamethylene carbonates, and the like. Also illustrative of the second recurring monomeric unit are those that are derived from monomers that polymerize by ring opening polymerization as, for example, substituted and unsubstituted beta, gamma, delta, omega, and other lactones such as those of the formula XXVII: 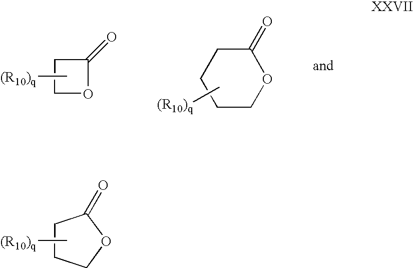
where R₁₀ is alkoxy, alkyl or aryl, and q is 0 to 3, wherein the open valencies are substituted with hydrogen atoms. Such lactones include caprolactones, valerolactones, butyrolactones, propiolactones, and the lactones of hydroxy carboxylic acids such as 3-hydroxy-2-phenylpropanoic acid, 3-hydroxy-3-phenylpropanoic acid, 3-hydroxybutanoic acid, 3-hydroxy-3-methylbutanoic acid, 3-hydroxypentanoic acid, 5-hydroxypentanoic acid, 3-hydroxy-4-methylheptanoic acid, 4-hydroxyocatnoic acid, and the like; and lactides such as 1-lactide, d-lactide, d,l-lactide; glycolide; and dilactones such as those of the formula XXVIII: 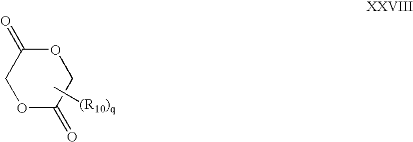
where R₁₀ and q are as defined above in Formula XXVII, and where the open valencies are substituted with hydrogen atoms. Such dilactones include the dilactones of 2-hydroxybutyric acid, 2-hydroxy-2-phenylpropanoic acid, 2-hydroxyl-3-methylbutanoic acid, 2-hydroxypentanoci acid, 2-hydroxy-4-methylpentanoic acid, 2-hydroxyhexanoic acid, 2-hydroxyoctanoic acid, and the like.

Illustrative of still further useful minor components are units derived from dioxepanones such as those described in U.S. Pat. No. 4,052,988 and U.K. Patent No. 1,273,733. Such dioxepanones include alkyl and aryl substituted and unsubstituted dioxepanones of the formula XXIX: 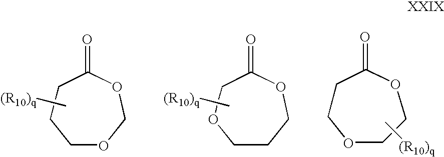
and monomeric units derived from dioxanones such as those described in U.S. Pat. Nos. 3,952,016, 4,052,988, 4,070,375, and 3,959,185, as for example, alkyl or aryl substituted and unsubstituted dioxanones of the formula XXX: 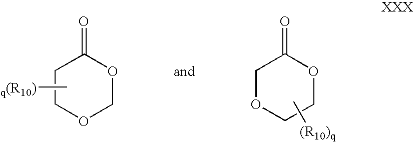
   wherein q is as defined above; R₁₀ is the same or different at each occurrence and are hydroxycarbonyl groups such as alkyl and substituted alkyl, and aryl or substituted aryl; and the open valencies are substituted with hydrogen atoms. Preferably R₁₀ is the same or different and are alkyl groups containing 1 to 6 carbon atoms, preferably 1 or 2 carbon atoms, and q is 0 or 1.

Suitable minor components also include monomeric units derived from ethers such as 2,4-dimethyl-1,3-dioxane, 1,3-dioxane, 1-,4-dioxane, 2-methyl-5-methoxy-1,3-dioxane, 4-methyl-1,3-dioxane, 4-methyl-4-phenyl-1,3-dioxane, oxetane, tetrahydrofuran, tetrahydropyran, hexamethylene oxide, heptamethylene oxide, octamethylene oxide, nonamethylene oxide, and the like.

Still further minor components include monomeric units derived from epoxides such as ethylene oxide, propylene oxide, alkyl substituted ethylene oxides such as ethyl, propyl, and butyl substituted ethylene oxide, the oxides of various internal olefins such as the oxides of 2-butene, 2-pentene, 2-hexene, 3-hexene, an like epoxides; and also including units derived from epoxides with carbon dioxide; and monomeric units derived from orthoesters or orthocarbonates such as alkyl or aryl substituted or unsubstituted orthoesters, orthocarbonates, and cyclic anhydrides which may optionally include one or more oxa, alkylaza, arylaza, and carbonyl groups of the formula XXXI: 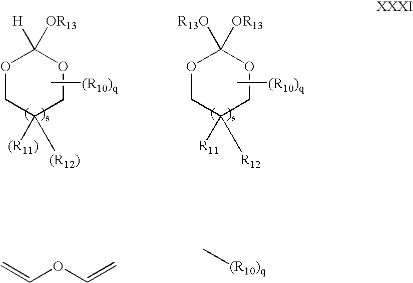
where q and R₁₀ are as described above, r is 0 to about 10, R₁₃ is the same or different at each occurrence and is alkyl or aryl, and R₁₁ and R₁₂ are the same or different and are hydrogen, alkyl or aryl.

Monomeric units derived from precursors and derivatives of lactides, lactones, dioxanones, orthoesters, orthocarbonates, anhydrides, and dioxepanones such as the various hydroxycarboxylic acids, substituted or non-substituted diacids such as oxa, aza, alkyl, aryl, hydroxy substituted oxacarboxylic acid acids, functionalized esters, and acid halide derivatives, and the like can also be used as the minor component.

Relative percentages of each of the recurring monomeric units that make up the copolymers of these embodiments can vary widely. The only requirement is that at least one type of recurring monomeric unit within the scope of Formula XXIIA be in the major amount, and that the other type of recurring unit or units be in the minor amount. As used herein, “major amount” is more than about 50 weight % based upon the total weight of all recurring monomeric units in the copolymer and “minor amount” is less than about 20 weight % based upon the total weight of all recurring monomeric units in the copolymer.

In addition, for certain applications, end-capping of these biopolymers can be desirable. End-capping can be accomplished by, for example, acylating, alkylating, silylating agents and the like.

In some embodiments, the random copolymers of these embodiments can be spun into fibers by any suitable fiber-forming technique, which fibers can then be fabricated in medical devices using conventional techniques. For example, once the random copolymers are formulated, the copolymers can formed into fibers by conventional processes such as spinning techniques, including melt, solution, dry, gel, and the like. Methods for spinning fibers from copolymers and polymers are well known in the art and will not be discussed further herein. Such fibers can be useful when these random copolymers are used to fabricate an implant comprising the polymer matrix alone, or when the random copolymers are used to form the core (described in more detail elsewhere herein).

The molecular weight of the random copolymer can vary widely depending upon the use of the copolymer formed. In general, the molecular weight of the copolymer is sufficiently high to allow its use in the fabrication of medical devices. Useful average molecular weight ranges of the copolymers for use in any particular situation will vary depending upon such features as the ultimate fiber properties and characteristics desired, such as modulus, tensile strength, bioresorption and biodegradation rates, and the like. In general, copolymer molecular weights useful for forming fibers are equal to or greater than about 10,000. Suitable average molecular weight ranges are about 10,000 to about 5,000,000, or about 20,000 to about 1,000,000, or about 30,000 to about 500,000.

Other polymeric components such as fillers and binders can be combined with the copolymers prior to and/or during the formation of fibers or devices, or subsequent to their formation. Suitable fillers and binders are known and will not be discussed further herein.

In addition, other degradable polymeric systems can be used according to the invention, such as polysaccharides and polypeptides. One of skill in the art, upon review of this disclosure, will readily appreciate the application of the inventive concepts to these additional degradable polymeric materials.

Formulation of Polymer Material

The polymer matrices of the invention are composed of at least one of the biodegradable polymers described herein, in combination with one or more bioactive agents. In some aspects, the biodegradable polymer comprises one or more of polyether ester copolymers (such as PEGT/PBT), terephthalate esters with phosphorus-containing linkages, and segmented copolymers with differing ester linkages, or polycarbonate-containing random copolymers.

Selection of the biodegradable polymer can be impacted by one or more considerations, such as, for example, the bioactive agent release rate desired for a particular application, the hydrophobicity of the polymer or polymers, and solvent compatibility. As an initial step, a bioactive agent is selected for treatment. Next a release rate that would provide a therapeutic or prophylactic dosage of the bioactive agent to a patient can be determined, based upon (for example) many of the considerations mentioned herein. Once a release rate is determined, this rate can be utilized to establish parameters for selection of the biodegradable polymer system to be utilized for the polymer matrix.

The bioactive agent release rate can be modulated in a number of ways. In some aspects, the relative amounts of biodegradable polymer(s) to bioactive agent(s) can be adjusted to further modulate the bioactive agent release rate. In some aspects, the composition of the biodegradable copolymer can be modified to modulate release rate. For example, when the biodegradable copolymer comprises an amphiphilic copolymer having hydrophilic units and hydrophobic units, the proportion of faster degrading polymer components (such as hydrophilic units) can be increased relative to the slower degrading polymer components (such as hydrophobic units) to provide a faster biodegradable composition release rate. In some embodiments, when most of the bioactive agent dosage is desired to be released over a long time period, the proportion of slower releasing polymer component can be increased relative to the faster releasing polymer component within the biodegradable copolymer.

Another selection parameter for the biodegradable polymer can be solvent compatibility. In some preferred aspects, the solvent system for the biodegradable polymer(s) and bioactive agent(s) are compatible.

The principle mode of degradation for many of the biodegradable polymers is hydrolysis. Degradation proceeds first by diffusion of water into the material followed by random hydrolysis, fragmentation of the material, and finally a more extensive hydrolysis accompanied by phagocytosis, diffusion, and metabolism. The hydrolysis can be affected by the size and hydrophilicity of the particular polymer material, the crystallinity of the polymer, and the pH and temperature of the environment. Once the polymer is hydrolyzed, the products of hydrolysis are either metabolized or secreted.

In one illustrative embodiment, when a relatively small-sized bioactive agent (for example, many antimicrobial agents, antiviral agents, and the like) is included in a PEGT/PBT polymeric material, the polyethylene glycol component of the copolymer preferably has a molecular weight in the range of about 200 to about 10,000, or in the range of about 300 to about 4,000. Also, the polyethylene glycol terephthalate is preferably present in the copolymer in an amount in the range of about 30 weight percent to about 80 weight percent of the weight of the copolymer, or in the range of about 50 weight percent to about 60 weight percent of the weight of the copolymer. According to these particular embodiments, the polybutylene terephthalate is present in the copolymer in an amount in the range of about 20 weight percent to about 70 weight percent of the copolymer, or in the range of about 40 weight percent to about 50 weight percent of the copolymer.

Suitable solvents that can be used to formulate the polymer matrix include, but are not limited to, chloroform, water, alcohol, acetone, acetonitrile, ether, methyl ethyl ketone (MEK), ethyl acetate, tetrahydrofuran (THF), dioxane, methylene chloride, xylene, toluene, N,N-dimethylformamide (DMF), dimethyl sulfoxide (DMSO), N,N-dimethylacetamide (DMAC), N-methylpyrrolidone (NMP), combinations of these, and the like.

To form polymer matrix with bioactive agent, the selected biodegradable polymer or polymers are combined and mixed with a bioactive agent. The bioactive agent can be present as a liquid, a finely divided solid, or any other appropriate physical form. The variety of different bioactive agents that can be used in conjunction with the biodegradable polymers of the invention is vast. Optionally, the polymer matrix can include one or more additives, such as diluents, carriers, excipients, stabilizers, or the like.

Upon contact with body fluids, the polymer matrix undergoes gradual degradation (mainly through hydrolysis) with concomitant release of the bioactive agent for a sustained or extended period. It is understood that bioactive active agent can diffuse out of the polymer mztrix as well, such that the mechanism for bioactive agent release is not dependent solely on degradation of the polymer matrix.

The inventive polymer matrices result in prolonged delivery (such as a period of several weeks) of therapeutically or prophylactically effective amounts of the bioactive agent. The therapeutically and/or prophylactically effective amount can be determined based upon such factors as the patient being treated, the severity of the condition, the judgment of the prescribing physician, and the like. In light of the teaching herein, those skilled in the art will be capable of preparing a variety of formulations.

In some aspects, the biodegradable composition includes polymers that are surface erodible and bulk erodible biodegradable materials. Surface erodible materials are materials in which bulk mass is lost primarily at the surface of the material that is in direct contact with the physiologic environment, such as body fluids. Bulk erodible materials are materials in which bulk mass is lost throughout the mass of the material; in other words, loss of bulk mass is not limited to mass loss that occurs primarily at the surface of the material in direct contact with the physiological environment.

In still further aspects, the composition of the copolymers themselves can be manipulated to provide desirable features. For example, when the copolymers include hydrophobic and hydrophilic portions, the relative amounts of these portions can be varied within the copolymer to provide a particular degradation rate. Likewise, the relative amounts of these portions can be varied within the copolymer to provide a desired bioactive agent release rate. It will be readily appreciated that bioactive agent release rate can be impacted by the degradation rate of the polymer, as well as the ability of the bioactive agent to diffuse from the polymer. Also, the ability for liquids (such as aqueous fluids) to permeate the polymer can impact the bioactive agent release rate and/or degradation rate. The present description provides various degradable polymer systems that can be utilized to deliver bioactive agent to limited access regions of the body, such as the eye. It will be appreciated that these illustrative degradable polymer systems can be manipulated to adjust bioactive release rate and/or degradation rate of the polymer.

Bioactive Agent

According to the invention, the polymer matrix includes a bioactive agent for sustained delivery of the bioactive agent to a treatment site. As used herein, “bioactive agent” refers to an agent that affects physiology of biological tissue. Bioactive agents useful according to the invention include virtually any substance that possesses desirable therapeutic and/or prophylactic characteristics for application to the implantation site.

For ease of discussion, reference will repeatedly be made to a “bioactive agent.” While reference will be made to a “bioactive agent,” it will be understood that the invention can provide any number of bioactive agents to a treatment site. Thus, reference to the singular form of “bioactive agent” is intended to encompass the plural form as well. Moreover, for purposes of discussion, reference will be made to association of the bioactive agent with a polymeric material composed of PEGT/PBT. However, it will be apparent upon review of this disclosure that the bioactive agent can be associated with any of the polymeric systems described herein. Further, the additives described herein are applicable to all polymer systems disclosed as well.

Exemplary bioactive agents include, but are not limited to, thrombin inhibitors; antithrombogenic agents; thrombolytic agents (such as plasminogen activator, or TPA: and streptokinase); fibrinolytic agents; vasospasm inhibitors; calcium channel blockers; vasodilators; antihypertensive agents; clotting cascade factors (for example, protein S); anti-coagulant compounds (for example, heparin and nadroparin, or low molecular weight heparin); antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, α-methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopoiamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl, betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, anti-VEGF antibody such as anti-VEGF fragment—ranibizumab (Lucentis)); dopamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Another group of useful bioactive agents are antiseptics. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Another group of useful bioactive agents are enzyme inhibitors. Examples of enzyme inhibitors include chrophonium chloride, N-methylphysostigmine, neostigmine bromide, physostigmine sulfate, tacrine HCL, tacrine, 1-hydroxymaleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor 1, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−)deprenyl HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+) p-aminoglutethimide tartrate, S(−)₃-iodotyrosine, alpha-methyltyrosine, L(−)alpha methyltyrosine, D,L(−)cetazolamide, dichlorophenamide, 6-hydroxy-2-benzothiazolesulfonamide, and allopurinol.

Another group of useful bioactive agents are anti-pyretics and antiinflammatory agents. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Biodegradable compositions can be formulated by mixing one or more bioactive agents with the polymers. The bioactive agent can be present as a liquid, a finely divided solid, or any other appropriate physical form. Typically, but optionally, the biodegradable composition will include one or more additives, such as diluents, carriers, excipients, stabilizers, or the like.

The particular bioactive agent, or combination of bioactive agents, can be selected depending upon one or more of the following factors: the application of the device (for example, subretinal implant, intraocular implant, and the like), the amount of the device composed of the polymer material (for example, percentage of the device fabricated of degradable material, inclusion of a biodegradable material as a coating on a surface of a core, as well as the amount of surface provided with the coating), the medical condition to be treated, the anticipated duration of treatment, characteristics of the implantation site, the number and type of bioactive agents to be utilized, and the like.

The concentration of the bioactive agent in the polymer matrix can be provided in the range of about 0.01% to about 75% by weight, or about 0.01% to about 50% by weight, based on the weight of the final polymer matrix. Preferably, the bioactive active agent is present in the polymer matrix in an amount in the range of about 75% by weight or less, preferably about 50% by weight or less. The amount of bioactive agent in the polymer matrix can be in the range of about 1 μg to about 10 mg, or about 100 μg to about 1000 μg, or about 300 μg to about 600 μg.

In some aspects, the concentration of bioactive agent can also be selected to provide a desired elution rate from the device. As discussed herein, some aspects of the invention provide methods including steps of selecting one or more bioactive agents to administer to a patient, determining a treatment course for a particular patient, and formulating the polymeric material to achieve the treatment course.

In some embodiments, a subretinal implant has a bioactive agent elution rate of at least 0.0001 μg per day, in other embodiments at least 0.001 μg per day, in other embodiments at least 0.01 μg per day, in other embodiments at least 0.1 μg per day, in other embodiments at least 1 μg per day, in other embodiments at least 10 μg per day. In some embodiments, an intraocular implant has an elution rate of at least 0.01 μg per day, in other embodiments at least 0.1 μg per day, in other embodiments at least 1 μg per day, in other embodiments at least 10 μg per day, in other embodiments at least 100 μg per day, and in other embodiments at lesat 1000 μg per day. The elution rate can vary and can be customized as desired for each type of eye condition treated, the nature of the ocular tissue being treated (for example, subretinal versus intraocular), the selected bioactive agent(s), the potency of bioactive agent(s), the size of the bioactive agent(s), and the severity of the condition being treated. In some aspects, the elution rate can be customized depending upon any physiological barriers that may exist between the implant site and the tissue to be treated. In general, it is desired to maximize the total bioactive agent(s) loading while maintaining mechanical integrity of the implant.

In one aspect, an intraocular implant including triamcinolone provides an elution rate in the range of about 1 to about 5 μg per day. One of skill in the art, upon review of the present disclosure, can readily determine a desirable elution rate for a particular implant, bioactive agent, condition, and the like.

The inventive implants can be utilized to deliver any desired bioactive agent or combination of bioactive agents to the eye, such as the bioactive agents described herein. The amount of bioactive agent(s) delivered over time is preferably within the therapeutic level, and below the toxic level. For example, a preferred target dosage for intraocular delivery of triamcinolone acetonide for use in treating diseases or disorders of the eye is preferably in the range of about 0.1 μg/day to about 10 μg/day, or in the range of about 0.5 μg/day to about 2 μg per day. Preferably, the treatment course is greater than 6 months, more preferably greater than one year. Thus, in preferred embodiments, the bioactive agent is released from the coated composition in a therapeutically effective amount for a period of 6 months or more, or 9 months or more, or 12 months or more, or 36 months or more, when implanted in a patient.

The inventive implants are formulated and configured to degrade upon implantation for a degradation period, and to release bioactive agent in a controlled manner for a release period. Generally speaking, the degradation period is longer than the bioactive agent. Put another way, the inventive implants release bioactive agent for a selected amount of time within the degradation period. In some aspects, the bioactive agent release period is 75% or less of the degradation period, or 70% or less of the degradation period, or 60% or less of the degradation period, or 50% or less than the degradation period, or 40% or less of the degradation period, or 30% or less of the degradation period, or 25% or less of the degradation period, or 20% or less of the degradation period. As mentioned, the degradation period comprises a longer period of time, relative to the bioactive agent release period. In some aspects, the degradation period comprises the amount of time a significant amount of the implant remains intact within the body (such as the amount of time a detectable, intact portion of the initial implant can be found at the implantation site). In some embodiments, the degradation period is 3 years or less, or 2 years or less, or 1 year or less, or 6 months or less. In some embodiments, the degradation period is in the range of 0.5 to 2 years.

In some aspects, the concentration of bioactive agent can be selected to provide a desired tissue concentration of bioactive agent at the treatment site. Given the site-specific nature of the inventive devices, methods and systems, it will be apparent that the tissue concentration of bioactive agent will be greater at the treatment site than at areas within the patient outside the treatment site. As discussed herein, this provides several benefits to the patient, such as reduced risk of toxic levels of the bioactive agent within the body, reduced risk of adverse affects caused by bioactive agent outside the treatment site, and the like. The location of the bioactive agent on or within the device and on or within the polymer can also affect tissue concentration of bioactive agent (for example, when substantially the entire implant includes bioactive agent, or selected portion(s) of the implant include bioactive agent). Moreover, inclusion of optional coating layers that contain bioactive agent can also impact tissue concentration of bioactive agent.

Implants of the invention provide significant advantages because they are designed for insertion, implantation and bioactive agent delivery directly at the desired treatment site. In some embodiments, the implants are designed for the treatment of disorders or diseases of the choroid and the retina. As such, the implants are inserted and implanted directly in the choroid, the retina or subretinal space, so as to deliver the bioactive agent precisely to the portion of the tissue being treated. In other embodiments, the implants are desired for treatment of disorders or dieases via intraocular routs. These implants are inserted and implanted in the vitreous of the eye. Such localized delivery to various targeted portions of the eye is efficient and delivers the bioactive agent substantially only to the portion of the eye being treated and does not deliver any significant amount of bioactive agent to healthy tissues. As used herein, the terminology delivery substantially only to the portion of the eye being treated is understood to mean that at least 5%, more preferably at least 10%, more preferably at least 20%, more preferably at least 30%, more preferably at least 40%, more preferably at least 50%, more preferably at least 60%, more preferably at least 70%, more preferably fit least 75%, more preferably at least 80% more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably all of the bioactive agent delivered by the implant is delivered to the treatment site. As used herein, the terminology “does not deliver any significant amount of bioactive agent to healthy tissues” is understood to mean that less than 95%, more preferably less than 90%, more preferably less than 80%, more preferably less than 70%, more preferably less than 60%, more preferably less than 50%, more preferably less than 40%, more preferably less than 30%, more preferably less than 20%, more preferably less than 15%, more preferably less than 10%, more preferably less than 5%, more preferably less than 4%, more preferably less than 3%, more preferably less than 2%, more preferably less than 1% of the total bioactive agent delivered by the implant.

This is in contrast to systemic, topical, and whole organ delivery mechanisms that have previously been used to treat diseases and disorders of the eye, as such mechanisms require the administration of significantly larger dosages of bioactive agents systemically, topically, orally or organ-wide so as to deliver a therapeutically effective amount of bioactive agent to the treatment site. For example, in order to administer a therapeutically effective dose of bioactive agent to treat a retinal disorder, approximately 1000 to 1,000,000 times the therapeutically effective dose may need to be administered systemically or orally. Not only does this result in the unnecessary waste of bioactive agent, but it also can cause undesirable toxicity and/or side effects from the delivery of such large amounts of bioactive agent. In some cases the systemic, oral and whole organ toxicity is so severe that a therapeutic dose may not be achievable by these conventional methods of administration. Further such delivery systems deliver bioactive agent to tissue and portions of the body that do not require the administration of such bioactive agent. In general, for example, such delivery systems deliver the bioactive agent to diseased and non-diseased portions of the eye. Likewise, whole organ delivery systems are also inefficient and require the delivery of substantially larger dosages of bioactive agents so as to provide a therapeutically effective amount of agent to the treatment site. As used herein, “local organ delivery system” is understood to mean a delivery system that delivers a bioactive agent generally to the organ being treated. Thus, for example, a local, whole visual/eye organ delivery system used to treat a retinal disease would deliver a bioactive agent to the diseased organ (the eye) rather than the diseased portion of the organ. The drawback with such systems is that the whole visual/eye organ receives a therapeutic level of drug even though only the diseased portion of the organ (e.g. the retina) actually requires treatment. Nonetheless, such local organ delivery systems deliver the bioactive agent to the entire eye, including the diseased tissues of the retina and healthy tissues of the eye. Further, such systems deliver the bioactive agent to a portion of the eye some distance away from the desired treatment site. As a result, the amount of bioactive agent that must be administered to the organ (the entire eye) may be in excess of the therapeutically effective dosage that would be required to treat the disorder or disease if the bioactive agent was delivered directly and only to the diseased eye tissues. For example, in order to administer a therapeutically effective dose to treat a retinal disorder, approximately 100 to 1000 times the therapeutically effective dose must be administered using whole organ delivery systems. The administration of bioactive agent to portions of an organ that do not require such administration may cause undesirable toxicity or side effects. For example, the delivery of a bioactive agent to an eye having a retinal disorder, but that is otherwise healthy, can potentially cause cataracts, raised intraocular pressure, and blurred vision from precipitated drug in the vitreous. While treatment of the retina and choroid of the eye has been discussed in particular, it is to be understood that the implants may similarly be used for the treatment of other ocular site specific disorders.

Because the present implants are more efficient than whole organ delivery systems they have a more discrete geometry than whole organ delivery systems. In particular, because whole organ delivery systems are required to hold and deliver a larger amount of bioactive agent to achieve the same therapeutic drug level at the disease site and, they are designed so as to maximize the amount of bioactive agent that can be held and delivered. Thus, for example, larger implants and/or implants with complex geometries (e.g. coiled or curved profiles) are required to provide a greater surface area and/or a larger interior reservoir for holding bioactive agent. The present implants need not hold such large quantities of bioactive agent and thus, need not possess larger and/or more complex geometries, although they may if desired.

Additives

In some aspects, it can be desirable to provide one or more additives to the biodegradable polymer matrix. Such additives can be included to impact the release of bioactive agent from the implant. Suitable additives according to these aspects include, but are not limited to, hydrophobic antioxidants, hydrophobic molecules, and hydrophilic antioxidants. Alternatively, additives can be included to impact imaging of the device once implanted. Illustrative additives will now be described in more detail.

Additives—Hydrophobic Antioxidant

In some embodiments, the biodegradable polymer matrix can optionally include at least one hydrophobic antioxidant. For example, when the polyetherester material (such as PEGT/PBT) includes a hydrophobic small-sized drug (such as, for example, a steroid hormone), the polymer matrix can include at least one hydrophobic antioxidant. Exemplary hydrophobic antioxidants that can be employed include, but are not limited to, tocopherols (such as α-tocopherol, β-tocopherol, γ-tocopherol, 6-tocopherol, ε-tocopherol, zeta₁-tocopherol, zeta₂-tocopherol, and eta-tocopherol), and ascorbic acid 6-palmitate. Such hydrophobic antioxidants can retard the degradation of the polyetherester copolymer material, and/or retard the release of the bioactive agent contained in the biodegradable polymer matrix. Thus, the use of a hydrophobic or lipophilic antioxidant can be desirable particularly to the formation of polymer matrices that include bioactive agents that tend to be released quickly from the polymer matrix, such as, for example, small drug molecules having a molecular weight less than 500 (in other words, the use of a hydrophobic or lipophilic antioxidant can slow release of the drug from the polymer matrix if desired).

In some embodiments, the antioxidant can improve drug stability as well. For example, inclusion of rapamycin in biodegradable ocular implants can be problematic, as rapamycin can be less stable than desired. Thus, inclusion of a hydrophobic antioxidant can, in some embodiments, improve the stability of rapamycin in a polymer matrix, and thus in the implant as a whole.

Typically, the hydrophobic antioxidant(s) can be present in the polymer matrix in an amount up to about 10 weight percent, or in the range of about 0.1 weight percent to about 10 weight percent of the total weight of the polymer matrix, or in the range of about 0.5 weight percent to about 2 weight percent.

Additives—Hydrophobic Molecule

In some embodiments, the polymer matrix can optionally include one or more hydrophobic molecules. For example, when the polyetherester material includes a hydrophilic small-sized bioactive agent (for example an aminoglycoside such as gentamycin), the polymer matrix can also include, instead of, or in addition to, the hydrophobic antioxidant herein described, at least one hydrophobic molecule. Illustrative hydrophobic molecules useful with the polymer matrix include cholesterol, ergosterol, lithocholic acid, cholic acid, dinosterol, betuline, and/or oleanolic acid. One or more hydrophobic molecules can act to retard the release rate of the bioactive agent from the polyetherester copolymer. Such hydrophobic molecules can prevent water penetration into the polymer matrix, but do not compromise the degradability of the polymer matrix. In addition, such molecules have melting points in the range of 150° C. to 200° C. or more. Therefore, a small percentage of these molecules increase the Tg of the polymer matrix, which decreases the matrix diffusion coefficient for the bioactive agent to be released. Thus, such hydrophobic molecules can, in some embodiments, provide for a more sustained release of a bioactive agent from the polymer matrix.

The hydrophobic molecule(s) can be present in the polymer matrix in an amount up to about 20 weight percent, or in the range of about 0.1 weight percent to about 20 weight percent, or about 1 weight percent to about 5 weight percent, based upon the total weight of the polymer matrix.

Additivies—Hydrophilic Antioxidant

When the polymer matrix (such as polyetherester copolymer) contains a protein, the copolymer can also optionally include a hydrophilic antioxidant. Examples of hydrophilic antioxidants include, but are not limited to, those having the following structural formula XXXII:
(X₁)_{Y}A-(X₂)_{Z} XXXII
wherein each of Y and Z is 0 or 1, wherein at least one of Y and Z is 1. Each of X₁ and X₂ is independently selected from the group consisting of compounds of the formula XXXIII: 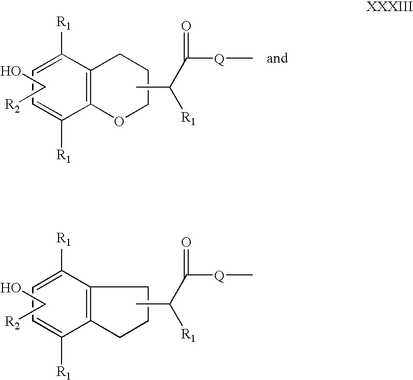
wherein each R₁ is hydrogen or an alkyl group having 1 to 4 carbon atoms, preferably methyl, and each R₁ is the same or different. R₂ is hydrogen or an alkyl group having 1 to 4 carbon atoms, preferably methyl. Q is NH or oxygen. Each of X₁ and X₂ can be the same or different. A is:
—(—R₃—O)ₙ—R₄ XXXIV
wherein R₃ is an alkyl group having 1 or 2 carbon atoms, preferably 2 carbon atoms; n is 1 to 100, preferably from 4 to 22; R₄ is an alkyl group having 1 to 4 carbon atoms, preferably 1 or 2 carbon atoms.

In one embodiment, one of Y and Z is 1, and the other of Y and Z is 0. In another embodiment, each of Y and Z is 1.

In yet another embodiment, R₃ is ethyl.

In a further embodiment, R₄ is methyl or ethyl.

In yet another embodiment, R₁ is methyl, R₂ is methyl, R₃ is ethyl, R₄ is methyl, one of Y and Z is 1 and the other of Y and Z is 0, Q is NH, n is 21 or 22, and the antioxidant has the following structural formula XXXV: 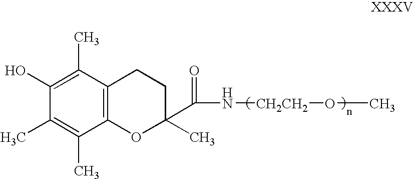
In another embodiment, the hydrophilic antioxidant has the following structural formula:
(X₃)_{Y}-A-(X₄)_{Z} XXXVI
wherein each of Y and Z is 0 or 1, wherein at least one of Y and Z is 1. Each of X₃ and X₄ is: 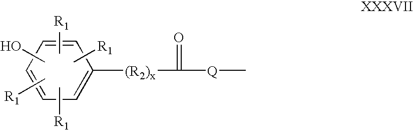
wherein each R₁ is hydrogen or an alkyl group having 1 to 4 carbon atoms, R₂ is an alkyl group having 1 to 4 carbon atoms, x is 0 or 1, and Q is NH or oxygen. Each R₁ is the same or different, and each of the X₃ and X₄ is the same or different. A is:
—(R₃—O—)ₙ—R₄ XXXVIII
wherein R₃ is an alkyl group having 1 or 2 carbon atoms, preferably 2 carbon atoms; n is from 1 to 100, preferably from 4 to 22; and R₄ is an alkyl group having 1 to 4 carbon atoms, preferably 1 or 2 carbon atoms.

In one embodiment, at least one, preferably two, of the R₁ moieties is a tert-butyl moiety. When two of the R₁ moieties are tert-butyl moieties, each tert-butyl moiety is preferably adjacent to the —OH group.

The hydrophilic antioxidant(s) can be present in the polymer matrix in an amount up to about 10 weight percent, or in the range of about 0.1 weight percent to about 10 weight percent, or about 1 weight percent to about 5 weight percent, based upon the total weight of the polymer matrix.

As discussed herein, the polymer matrix can include one or more hydrophobic antioxidants, hydrophobic molecules, and/or a hydrophilic antioxidants in the amounts described herein. The type and precise amount of antioxidant and/or hydrophobic molecule employed can be dependent upon the molecular weight of the bioactive agent (protein), as well as properties of the polymer matrix itself. If the polymer matrix includes a large peptide or protein (such as, for example, insulin), the matrix can also optionally include a hydrophilic antioxidant such as those described herein and in the amounts described herein, and can also include polyethylene glycol having a molecular weight in the range of about 1,000 to about 4,000, in an amount in the range of about 1 weight percent to about 10 weight percent, based upon the total weight of the copolymer.

Additives—Imaging Materials

In some embodiments, the polymer material can further include imaging materials. For example, materials can be included in the polymer material to assist in medical imaging of the device once implanted. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in any fabrication method or absorbed into or sprayed onto the surface of part or all of the implant. The degree of radiopacity contrast can be altered by controlling the concentration of the radiopacifier within or on the implant. Radiopacity can be imparted by covalently binding iodine to the polymer monomeric building blocks of the elements of the implant. Common radio opaque materials include barium sulfate, bismuth subcarbonate, and zirconium dioxide. Other radio opaque materials include cadmium, tungsten, gold, tantalum, bismuth, platinum, iridium, and rhodium. In some embodiments, iodine can be employed for both its radiopacity and antimicrobial properties. This can be useful for detection of medical devices that are implanted in the body (that are emplaced at the treatment site) or that travel through a portion of the body (that is, during implantation of the device). Paramagnetic resonance imaging, ultrasonic imaging, x-ray means, fluoroscopy, or other suitable detection techniques can detect medical devices including these materials. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep. 1996); other vapor phase chemicals useful for ultrasonic imaging can be found in U.S. Pat. No. 6,261,537 (Issued 17 Jul. 2001).

Thus, additives can be included in the polymer matrix to control release of bioactive agent, impact degradation of the polymer matrix, and/or impact imaging of the device once implanted. In some aspects, release of bioactive agent can also be impacted by modification of the polymer matrix itself. Another technique for impacting release of bioactive agent can involve modifying the configuration of the device.

Additives—Excipients

In some aspects, the polymer matrix can include an excipient. A particular excipient can be selected based upon its melting point, solubility in a selected solvent (such as a solvent that dissolves the biodegradable polymer and/or the bioactive agent), and the resulting characteristics of the composition. Excipients can comprises a few percent, about 5%, 10%, 15%, 20%, 25%, 30%, 40%, 50%, or higher percentage of the particular polymer in which it is included.

Buffers, acids, and bases can be incorporated in the polymer matrix to adjust pH. Agents to increase the diffusion distance of bioactive agents released from the polymer matrix can also be included. Illustrative excipients include salts, PEG or hydrophilic polymers, and acidic compounds.

Thus, additives can be included in one or more polymers comprising the biodegradable polymer matrix to assist in controlling release of bioactive agent, impacting degradation of the polymer matrix, and/or impacting imaging of the device once implanted.

Optionally, the biodegradable polymer itself can be modified to affect the degradation rate and release rate of a bioactive agent. For example, the polymer can be modified by replacing components (monomeric units) with a particular hydrophobicity with a component (monomeric unit) that has a differing hydrophobicity. In some aspects, the individual monomeric units of biodegradable polymers that comprise copolymers can be modified to achieve desired degradation rate and/or release of bioactive agent. In one illustrative embodiment, PEGT/PBT can be formulated to include a protein having a molecular weight greater than 10,000. In this instance, the polyethylene glycol component of the copolymer can have a molecular weight in the range of about 1,000 to about 20,000. The polyethylene glycol terephthalate can be present in the copolymer in an amount in the range of about 30 weight percent to about 90 weight percent, or about 60 weight percent to about 70 weight percent of the weight of the copolymer. The polybutylene terephthalate can be present in the copolymer in an amount in the range of about 10 weight percent to about 70 weight percent, or about 30 weight percent to about 40 weight percent of the weight of the copolymer.

These concepts can be applied to the other degradable polymer systems described herein as well. The composition of the copolymer can be modified whether additives are included in the copolymer or not.

Core

Optionally, the implant comprises a biocompatible core material that is coated with a coating layer of a polymer matrix-bioactive material (a polymer matrix including one or more bioactive agents). A “coating” as described herein can include one or more “coated layers,” each coated layer including one or more coating components (such as polymeric components, and/or bioactive agent). When more than one coated layer is applied to the surface of a device, it is typically applied successively. For example, a coating is typically formed by dipping, spraying, or brushing a coating material on a device to form a layer, and then drying the coated layer. The process can be repeated to provide a coating having multiple coated layers, wherein at least one layer includes a bioactive agent. Typically (but not always), at least the coated layer located nearest the device surface includes bioactive agent. In some aspects, more than two coated layers can be present. Such other layers can be the same or different than the first coated layer and/or second coated layer. Optionally, topcoats and/or priming layers can be included the coatings, and these topcoats and/or priming layers can be provided with or without bioactive agent. The suitability of the coating for use with a particular medical device, and in turn, the suitability of the application technique, can be evaluated by those skilled in the art, given the present description.

Reference is made to FIGS. 2-4 for illustrative implants including a core having a polymer matrix as a coating. In alternative embodiments, implants having a configuration as shown in FIGS. 5-10 can include a core. In these embodiments, the body member includes a core and a polymer matrix, the polymer matrix forming a coating on a surface of the core, or contained in a lumen within the body member. Reference to a “core” herein is thus intended to encompass any of the configurations described herein. For purposes of discussion herein, including (but not limited to) discussion of the embodiments illustrated in FIGS. 5-10, the terms “core” and “body member” can be used interchangeably. In some aspects, the core can be fabricated of any of the biodegradable materials described herein as suitable for the coating. Some illustrative biodegradable core materials include polyglycolic acid (PGA), polydioxanone (PDO), surgical gut (for example, derived from serosal layer of bovien or sheep intestines), polylactic acid (PLA), polyglyconate (or polytrimethylene carbonate), polyglactin, and polyglecaprone.

In other aspects, the core can be fabricated of a biostable material. Representative biostable polymers include polyurethanes, silicones, polyesters, polyolefins (such as polyethylene or polypropylene), polyisobutylene, acrylic polymers, vinyl halide polymers, polyvinyl ethers, polyvinyl methyl ether, polyvinylidene halides, polyacrylonitrile, polyvinyl ketones, polyvinyl aromatics, polyvinyl esters (e.g., poly(alkyl(meth)acrylates) such as poly((methyl)methacrylate) or poly((butyl)methacrylate)), polyvinyl amides, polyamides, polycaprolactam, polycarbonates, polyoxymethylenes, polyimides, polyethers, polyurethanes, rayon, rayon-triacetate, cellulose acetate, cellulose butyrate, cellophane, cellulose nitrate, cellulose propionate, cellulose ethers, carboxymethyl cellulose and copolymers (such as polyethylene vinyl acetate) and blends of the above polymers.

Non-polymer based biocompatible materials may be used as the core of an implant of the invention. Representative examples include include titanium-nickel alloy wire (e.g., Nitinol wire, commercially available from Nitinol Devices and Components, Fremont, Calif.), titanium alloys, nickel-cobalt base alloys, stainless steel, cobalt-chromium alloys, and biodegradable magnesium alloys. It is to be understood that the core material is not limited to the examples provided herein and can be any conventional material used in implant devices.

The size, geometry and materials used in forming the core can be selected to provide the desired characteristics. For example, thinner cores can be used to provide less rigidity and to allow for thicker coatings of polymer matrix-bioactive material, thereby maximizing the volume of bioactive agent loading in the implant. Further, the material forming the core can be selected to provide the desired rigidity/flexibility. Still further, core materials can be selected so as to facilitate the ability of the polymer matrix-bioactive material to adhere as a coating to the core material. Still further, the surface of the core material could be primed, roughened, or chemically modified to further facilitate the ability of the polymer matrix-bioactive material to adhere to the core material.

When the core is fabricated of polymer materials, the core can be formed by any known method for forming polymeric devices such as filaments, discs, and the like.

For example, in one illustrative embodiment, PEGT/PBT copolymer is utilized to fabricate a filament. The filament can be formed by any number of well-known methods including melt extrusion or solvent extrusion. The extrusion procedure can be varied depending upon the stability of bioactive agent (if any) to be included in the core. In the solvent extrusion method, bioactive agent and polymer solutions are prepared at high concentrations (approximately 1 g/ml), and are forced through a narrow syringe needle. Filament thickness can be varied easily between approximately 150 to 1000 μm.

In another aspect, the invention provides methods for preparing an implantable device. In some embodiments the method comprises the steps of: (a) dissolving one or more polymers in a solvent to form a complex fluid; (b) adding at least one bioactive agent to the complex fluid to produce a homogeneous solution of the one or more bioactive agents and/or a solution with a dispersed phase of one or more bioactive agents; (c) optionally drying the complex fluid to a solid form; (d) optionally heating the solid form to a temperature just below the melting point of the polymer(s); and (e) forming the implant device out of the solution of (b) or the solid form of (c).

In some embodiments, the methods for preparing an implantable device comprise use of a low temperature process (e.g., from about 20° C. to about 100° C., more preferably from about 50° C. to about 90° C.) to form implants. In one embodiment, the method comprises a process that involves homogenously mixing the polymer and one or more bioactive agents in solvent, drying, and melt-extrusion-drawing the prepared solid-form into the implant shape. More specifically, the method comprises: dissolving one or more polymers in a suitable solvent solution to produce a complex fluid; adding one or more bioactive agents to the complex fluid to produce a homogeneous solution of one or more bioactive agents and/or a solution with a dispersed phase of one or more bioactive agents; drying the solution to a solid form; heating the solid form to a temperature below the melting point of the polymer (e.g., about 1° C. to about 5° C. below the melting point); forming the implant device out of this semi solid; and shaping the filament into the desired shape by drawing it into a lengthy filament and mechanically sectioning it into a fixed length. Bending the implant device can add curvature.

In other embodiments the complex fluid is not dried to a solid form. In these embodiments, heating may not be required during the forming step because of the presence of the solvent in the complex fluid.

The steps of forming the implant device and shaping the filament into the desired shape can be accomplished by a variety of conventional methods for forming and shaping a device out of a solid. For example, the solid form may be processed by melt-extrusion-drawing (applying tensile force) to form the solid into the desired shape and thickness. The length can be modified by cutting the device with any conventional cutting tool. The distal and/or proximal ends of the implant can be shaped by cutting, sanding, and other methods for forming tapered, rounded, beveled and other desired end shapes.

In some embodiments, the implant is fabricated by: solubilizing polycaprolactone in chloroform at a temperature below boiling, overnight, under still or continuous stirring conditions; adding a bioactive agent to the solution in a ratio that preferably ranges from 1:99 to 70:30 (wt. bioactive agent:wt. polymer) depending on the prepared formulation; allowing the solvent to evaporate under still or stirring conditions after the solution becomes translucent or dispersed; transferring the solid-form of the loaded polymer to an extrusion device; heating the extrusion device to about 50° C. to about 90° C., depending on the molecular weight of the polycaprolactone (Mₙ=3,000 to 120,000), such that the polymer temperature approaches the melt temperature but does not exceed it; drawing the solid form to its desired geometry once the extrusion device reaches the desired sub melt temperature; shaping the implant to the desired implantation length after the temperature of the drawn implant falls. In another embodiment, filaments can be made by subjecting the polymeric melt to extrusion molding to produce filaments having a desired diameter (for example, in the range of 1 to 2 mm). The filaments can be drawn (to induce orientation and self-reinforcement) at a temperature (T) of Tm>T>Tg (where Tg is polymer glass transition temperature and Tm is polymer melting temperature) to a specified diameter (for example, 1 mm). If it is desired to fabricate a device consisting of multiple filaments in a woven configuration, the filaments can then wound in a hot state around a substrate (such as a metal pipe having a diameter of 5 mm), cooled, and removed from the surface of the substrate. The devices so formed can then be immersed in buffer solutions, if desired, to maintain pH in a desired range.

In some aspects, the core can be formed by melt spinning. Spinning from solution can be used in lieu of high temperature (about 190° C.) melt extrusion. Methylene chloride (b.p. 55° C.) is a preferred solvent for use in such a process. The solvent can be removed during the spinning process by: (i) evaporating solvent from the protofibers descending from a spinneret with warm air (dry spinning) or (ii) squirting the polymer solution into a liquid bath, the liquid being a non-solvent for the polymer but miscible with the solvent in the spinning solution, for example, methyl alcohol (wet spinning).

In some embodiments, the implant (with or without the core material) can further include a layer of material that modifies the bioactive agent release rate characteristics. For example, a thin layer of polycaprolactone can be coated on the implant. Such a polycaprolactone layer can also provide a degradation rate-controlling barrier, protection of the bioactive agent from environmental degradation prior to implantation, or even delay the time point of release of the drug.

When the implant comprises a core, the implant can be fabricated by applying a coating composition comprising one or more polymers and one or more bioactive agents over at least a portion of the outer surface of a core material. Typically, biodegradable coatings are provided to a surface of the core after fabrication of the core. In this way, stability and activity of the bioactive agent can preferably be protected from the conditions of fabrication of the structural portion (core) of the device (conditions such as heat, pressure, and the like).

The coatings of the invention can be applied to a surface in a manner sufficient to provide a suitably durable and adherent coating on the surface. Typically, the coatings are provided in a manner such that they are not chemically bound to the surface. Rather, the coatings can be envisioned as encapsulating the device surface. Given the nature of the association between the coating and surface of the core, it will be readily apparent that the coatings can be applied to virtually any surface material to provide a suitably durable and adherent coating. Moreover, in some embodiments, a suitable surface pretreatment can be utilized, to enhance the association between the coating and the device surface.

The coating composition can be applied to the outer surface of the core using any suitable method. For example, the coating composition may be applied by dipping, spraying, and other known methods for applying coating compositions to substrates. The suitability of the coating composition for use on a particular material can be evaluated by those skilled in the art.

In some embodiments, the coating composition is applied to the core utilizing a precision coating system wherein the coating material is atomized ultrasonically (an ultrasonic coating system). Exemplary ultrasonic coating systems and methods are described in U.S. Published Application 2004/0062875 (Chappa et al.); and in U.S. application Ser. No. 11/102,465, filed Apr. 8, 2005 and entitled “Medical Devices and Methods for Producing Same.”

In some embodiments, a core (such as a TiNi wire) to be coated is mounted in a pin vise, or similar device, that is capable of rotating the device about its longitudinal axis. The device is rotated and the ultrasonic spray head is passed back and forth relative to the rotating core.

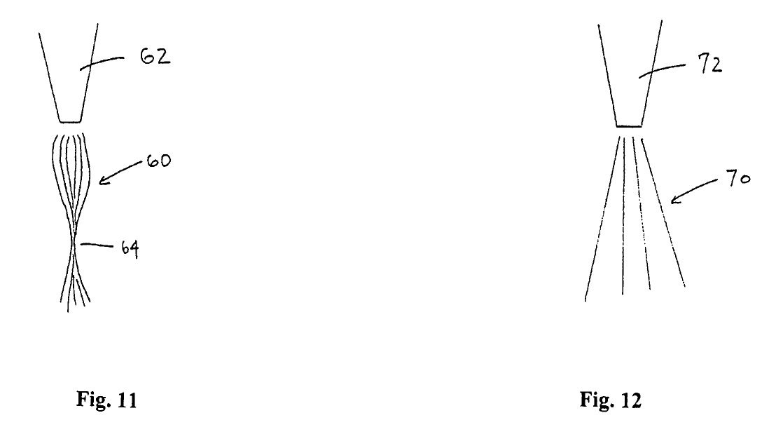

Ultrasonic coating systems can produce a spray stream that narrows down as it moves away from the coating head. Referring to FIG. 11, the spray stream 60 narrows as it travels away from the coating head 62 before passing through a focal point 64 (or point of smallest spray stream diameter) before starting to expand. In an embodiment, the focal point has a cross-sectional diameter of about 0.5 mm to about 1.0 mm. In contrast, other types of spray systems frequently produce a spray stream that continuously expands in diameter as it leaves the spray head. Referring to FIG. 12, the spray stream 70 continues to get wider as it travels away from the coating head 72.

Ultrasonic coating systems can be used to coat a core with a large degree of accuracy, particularly where the core to be coated is positioned at or near the focal point of the spray stream. This is because the spray stream has a relatively small cross-sectional area at or near the focal point because the spray stream has a relatively small amount of spray droplets that are outside of the focal point. As the spray stream has a relatively small cross-sectional area, the position of the spray stream with respect to the core to be coated must be moved if a broader area of the core is to be covered with a coating. Either the core or the spray head may be moved to cover a broad area.

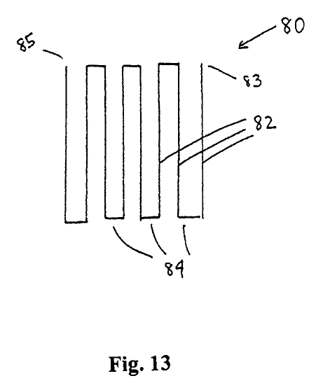

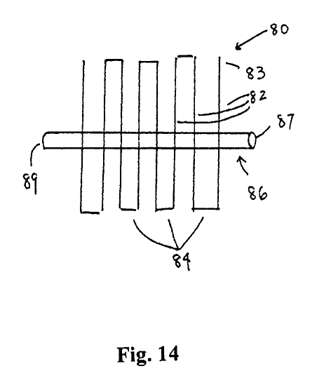

In an embodiment, the ultrasonic spray head is moved back and forth over the rotating core in a grid-like pattern. By way of example, an exemplary grid-like pattern 80 is shown in FIG. 13. The grid-like pattern starts at point 83 and ends at point 85. The grid like pattern has a series of transverse sweeps 82 and longitudinal movements 84. Depending upon the length of the longitudinal movements 84, any number of transverse sweeps can be used to cover the length of a given coating layer. In embodiments of the invention, the grid-like pattern 80 includes between 3 and 100 transverse sweeps 82. In embodiments of the invention, the grid-like pattern 80 includes between 3 and 100 longitudinal movements 84. Referring now to FIG. 14, grid-like pattern 80 is superimposed over an exemplary core material 86 having distal end 87 and proximal end 89 to illustrate how core material 86 would be coated with reference to the grid-like pattern 80.

The length of the longitudinal movements can be varied depending upon various factors including the cross-sectional diameter of the spray pattern as it meets the surface of the device to be coated. It has been found that when the longitudinal movements are greater than a desired amount, and when the grid-like pattern is followed from the same place on each pass, the surface of the coating may become bumpy. The specific limit on the size of the longitudinal movements will depend upon a number of factors including the diameter of the spray pattern and the relative spray density of various parts of the spray pattern.

In some embodiments, the ultrasonic coating head follows the grid-pattern multiple times (that is, multiple passes) in order to deposit a coating layer onto a core. On each pass, an amount of the coating layer is deposited. Thus, the precise number of passes made by the ultrasonic coating head can be changed based on the total coating thickness desired. In some embodiments, the mass of the coating layer comprises between about 10 μg and about 1000 μg dry weight. In other embodiments, the mass of the coating layer comprises between about 50 μg to about 300 μg dry weight.

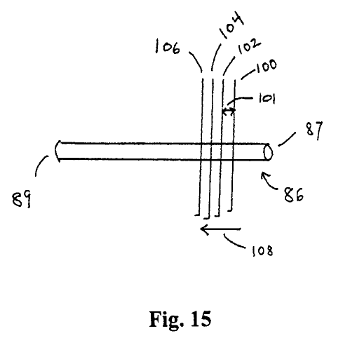

In some embodiments, the same longitudinal starting position is used with respect to the core for each pass of the ultrasonic coating head. For example, for each pass, the ultrasonic coating head would start at the same longitudinal point and follow the same pattern. In other embodiments, the longitudinal starting position of the ultrasonic coating head may change with each additional pass. Referring to FIG. 15, the first transverse sweep of the first pass may start at point 100. Then, the first transverse sweep of the second pass may start at an offset position 102 that is offset at a distance 101 from starting point 100. Similarly, the first transverse sweep of the third pass and fourth pass begin at points 104 and 106, respectively. This technique of moving the starting position in the direction of arrow 108 can be used to extend the distance over which the coating builds up to its full thickness thereby controlling the slope of the transition segment of the coating layer. By way of example, the offset distance between successive passes could be 0.5 mm. This would generally result in a longer transition segment with a lower slope in comparison with a coating layer that was applied with an offset between successive passes of less than 0.5 mm, for example 0.2 mm. The slope of the transition segment may be desirably low (for example, less than about 1.0) when the implant will undergo stresses (for example, frictional stresses) that may result in delamination or failure of the coating. The slope of the transition segment may be desirably high (for example, greater than about 1.0) where it is desired to maximize the amount of the coating layer on the implant. The proximal and distal transition segments of the coating layer may have slopes that are the same or different. For example, in some embodiments, the distal transition segment has a slope that is less than the proximal transition segment.

In some embodiments, the coating comprises at least two layers, wherein each layer comprises the same composition, or comprises different compositions. In one such embodiment, a first layer having either bioactive agent alone, or bioactive agent together with one or more of the biodegradable polymers is applied, after which one or more additional layers are applied, each with or without bioactive agent. These different layers, in turn, can cooperate in the resultant composite coating to provide an overall release profile having certain desired characteristics, and is particularly preferred for use with bioactive agents having high molecular weight. According to the invention, the composition of individual layers of the coating can include any one or more of the following: one or more bioactive agents, and/or a biodegradable polymer, as desired.

Preferably, the coating composition is applied to the core of the implant in one or more applications. The method of applying the coating composition to the body member is typically governed by such factors as the geometry of the device and other process considerations. The coated composition can be subsequently dried by evaporation of the solvent. The drying process can be performed at any suitable temperature, (for example, room temperature or elevated temperature), and optionally with the assistance of vacuum.

In some preferred embodiments, the coating composition is applied to the core under conditions of controlled relative humidity. As used herein, “relative humidity” is the ratio of the water vapor pressure (or water vapor content) to the saturation vapor pressure (or the maximum vapor content) at a given temperature of the air. The saturation vapor pressure in the air varies with air temperature: the higher the temperature, the more water vapor it can hold. When saturated, the relative humidity in the air is 100% relative humidity. According to some embodiments of the invention, the coating composition can be applied to the core under conditions of increased or decreased relative humidity as compared to ambient humidity.

According to the invention, humidity can be controlled in any suitable manner, including at the time of preparing and/or applying the coating composition to the body member. For example, when humidity is controlled at the time of preparing the coating composition, the water content of the coating composition can be adjusted, before and/or after the coating composition is applied to the body member. When humidity is controlled at the time of applying the coating composition, the coating composition can be applied to the body member in a confined chamber or area adapted to provide a relative humidity that differs from ambient humidity. Generally, it has been found that applying coating compositions under conditions of increased humidity will typically accelerate release of the bioactive agent, while applying coating compositions under conditions of decreasing humidity levels will tend to decelerate release of the bioactive agent. As contemplated in the invention, even ambient humidity can be considered “controlled” humidity if it has been correlated with and determined to provide a corresponding controlled release of the bioactive agent.

Moreover, and particularly when coating a plurality of coating compositions onto the body member of the controlled delivery device to provide the final coated composition, humidity can be controlled in different ways (for example, using a controlled environment as compared to adjusting the water content of the coating composition) and/or at different levels to provide a desired release profile for the resulting coated composition. As described previously, a coated composition can be provided using a plurality of individual steps or layers of coating composition, including, for instance, an initial layer having only bioactive agent (or bioactive agent with one or both polymers), over which is coated one or more additional layers containing suitable combinations of bioactive agent and polymeric material, the combined result of which is to provide a coated composition of the invention.

Thus, in preferred embodiments, the invention provides the ability to reproducibly control the release of a bioactive agent from a controlled delivery device.

In some embodiments, a plurality of coating compositions and corresponding coating steps can be employed, each with its own controlled humidity (when desired), in order to provide a desired combination of layers, each with its corresponding release profile. Those skilled in the art will appreciate the manner in which the combined effect of these various layers can be used and optimized to achieve various effects in vivo.

Other coating techniques can be utilized for providing a coating on a core. In some embodiments, the implant can be immersed in a biodegradable composition solution to form a coating. In other embodiments, the biodegradable coating composition is spray coated onto a surface of an implantable device.

The inventive biodegradable coating compositions can be applied to any desired portion of the device surface. For example, in some embodiments, the biodegradable coating composition can be provided on the entire surface of the device. In other embodiments, only a portion of the device can include the biodegradable coating composition. The portion of the device carrying the biodegradable coating composition can be selected based upon such factors as the application of the device, the amount of bioactive agent to be applied at a treatment site, the number and types of bioactive agents to be delivered, and like factors.

Moreover, each coated layer of the biodegradable coating composition can be provided on the surface of the device in any number of applications. The number of applications can be selected to provide individual coated layers of suitable thickness, as well as a desired total number of multiple coated layers of biodegradable composition, as desired. In some embodiments, the number of applications can be controlled to provide a desired overall thickness to the polymer coating. Generally, the thickness of the coating is selected so that it does not significantly increase the profile of the device for implantation and use within a patient.

Typically, for use in connection with subretinal implants, the overall thickness of the biodegradable coating composition is up to about 400 μm, or up to 350 μm, or up to 300 μm, or up to about 250 μm, or up to about 200 μm, or up to about 150 μm, or up to about 100 μm. When a core is included, the overall diameter of the implant can be up to about 500 μm, or up to about 450 μm, or up to about 400 μm, or up to about 350 μm, or up to about 300 μm, or up to about 250 μm, or up to about 200 μm, or up to about 150 μm, or up to about 100 μm. In some aspects, the overall diameter of a subretinal filament is in the range of about 0.1 to about 300 μm, or in the range of about 100 μm to about 250 μm.

Typically, for use in connection with intraocular implants, the final coating thickness of the coated composition on the controlled delivery device is up to about 100 μm, or in the range of about 0.1 μm to about 100 μm, or in the range of about 5 μm to about 60 μm, or in the range of about 10 μm to about 40 μm. This level of coating thickness is generally effective to provide a therapeutically effective amount of bioactive agent to the implantation site under physiological conditions. The final coating thickness can be varied, and at times be outside the preferred ranges identified herein, depending upon such factors as the total amount of bioactive agent to be included in the coated composition, the type of bioactive agent, the number of bioactive agents to be included, the treatment course, the implantation site, and the like.

In some aspects, the composition of individual layers of the coating can be the same or different, as desired. For example, each layer can include bioactive agent and/or one or more biodegradable polymers. In some aspects, the device can include an outer coating that comprises a lubricious coating. In these aspects, a lubricious surface is provided at the surface that encounters body tissue during implantation and use of the device. Such lubricious surfaces can reduce complications of the implantation procedure by reducing or minimizing adhesion of vitreal tissue.

When the implantable devices of the invention include a coating, the coating can be provided with the same or different bioactive agent or agents as the core. Moreover, when the coating is composed of multiple layers of degradable polymer material, each individual layer, or groupings of layers, can include different bioactive agents. For example, in a subretinal filament, a coating can include an antibiotic (such as neomycin) to reduce or prevent infection, an inner layer with an anti-inflammatory (such as a steroid, for example triamcinolone or dexamethasone) to reduce or prevent inflammatory response, and the core can include anti-angiogenic factor to treat the underlying disease or disorder (for example, to prevent or reduce formation of new blood vessels).

When more than one component is utilized to form a core, the polymer matrix coating can be provided in a number of ways. For example, when multiple fibers or filaments are combined to form a core (such as by winding or molding the fibers or filaments to form a core), provision of the polymeric coating composition to the core can be achieved in any desirable manner. For example, each individual strand can be provided with a polymeric coating composition prior to twisting the strands to form the core. Alternatively, the individual, uncoated, strands can be twisted to form the core, and the formed core can be provided with the polymeric coating composition.

In another embodiment, the surface area of the core can be increased by including surface configurations on the core. According to these embodiments, any suitable type of surface configuration can be provided to the core, such as, for example, dimples, pores, raised portions (such as ridges or grooves), indented portions, and the like. Surface configuration can be accomplished by roughening the surface of the material used to fabricate the core. In one such embodiment, the surface of the body member is roughened using mechanical techniques (such as mechanical roughening utilizing such material as 50 μm silica), chemical techniques, etching techniques, or other known methods. In other embodiments, surface configuration can be accomplished by utilizing a porous material to fabricate the core. Examples of porous material are described elsewhere herein. Alternatively, materials can be treated to provide pores in the material, utilizing methods well known in the art. In still further embodiments, surface configuration can be accomplished by fabricating the core of a machined material, for example, machined metal. The material can be machined to provide any suitable surface configuration as desired, including, for example, dimples, pockets, pores, and the like.

In preferred embodiments, surface configuration of the core can provide advantages, such as, for example, increased surface area of the core for application of the polymeric coating composition, increased durability of the device, increased tenacity of the polymeric coating composition to the core (for example, by virtue of a roughened surface, increased surface area for adherence, and the like), enhanced removability of the device after a desired treatment duration, and the like.

When the implant is provided in a configuration similar to that depicted in FIGS. 5-10, increased device surface area can be provided by utilizing a body member configured as a threaded shaft that is tapered or untapered, as desired. Such threaded shaft embodiments are similar to a typical wood screw. The threaded shaft can be fabricated using any suitable techniques, such as molding or machining the threads of the shaft. Further, the threading on the shaft can be a continuous spiral thread that runs continually from the proximal to the distal end of the body member, or the threading can be provided as noncontiguous rings about the body member. Although these particular embodiments can require a larger incision site for implantation of the device in a patient, in some applications, the increased surface area provided by the threaded shaft (discussed in more detail herein) can outweigh the larger incision required.

The core can include surface configurations along its entire length, or only a portion of the length of the body member, as desired.

The coated composition is provided in contact with at least a portion of the core of the device. In some embodiments, for example, it can be desirable to provide the coated composition in contact with the entire surface of the core. Alternatively, the coated composition can be provided on a portion of the core (such as, for example, an intermediate portion of the core located between the proximal and distal ends thereof). In some preferred embodiments, for example, it can be desirable to provide the coated composition in contact with a portion of the core that does not include a sharp distal tip of the core. This can be desirable, for example, to reduce risk of delamination of the coated composition at the sharp tip and/or to maintain the sharpness of the tip. The amount of the core that is in contact with the coated composition can be determined by considering such factors as the amount of bioactive agent to be provided at the implantation site, the choice of polymer matrix for the coated composition, the characteristics of the implantation site, risk of delamination of the coated composition, and the like. For example, in some embodiments, it can be desirable to provide the coated composition on portions of the core other than the proximal and distal ends of the device, so as to reduce risk of delamination upon implant and/or explant of the device. Optionally, such delamination can also be minimized, in some embodiments, by providing a stepped coating thickness, such that the coating thickness decreases towards the proximal and/or distal ends of the core. In still further optional embodiments, the core can be provided with a coated composition at its distal and/or proximal ends that differs from the composition of the coating at other portions of the core. One example of such an embodiment includes a core having a lubricious coating at the distal and/or proximal end of the core, with a different coated composition in the intermediate portion of the core that is located between the proximal and distal ends of the core. Utilizing the concepts described herein, one of skill in the art can determine the amount of core to be provided in contact with the coated composition, and/or the composition of coated composition provided at one or more distinct regions of the core, as desired.

Thickness of the coated composition on the controlled delivery device can be assessed using any suitable techniques. For example, portions of the coated composition can be delaminated by freezing the coated controlled delivery device, for example, utilizing liquid nitrogen. The thickness at the edge of a delaminated portion can then be measured by optical microscopy. Other visualization techniques known in the art can also be utilized, such as microscopy techniques suitable for visualization of coatings having the thickness described herein of the invention.

The overall weight of the coated composition upon the surface of the controlled delivery device is typically not critical. The weight of the coated composition attributable to the bioactive agent can be in the range of about 1 μg to about 10 mg of bioactive agent per cm² of the surface area of the controlled delivery device. In some embodiments, the surface area can comprise all or a portion of the body member 2 of the device. In alternative embodiments, the surface area can comprise the body member 2 and the cap 8 of the device. Preferably, the weight of the coated composition attributable to the bioactive agent is in the range of about 0.01 mg to about 10 mg of bioactive agent per cm² of the surface area of the controlled delivery device. This quantity of bioactive agent is generally effective to provide adequate therapeutic effect under physiological conditions. As used herein, the surface area is the macroscopic surface area of the device.

According to the invention, the device can optionally further include a sheath that is configured to surround and enclose the device. Generally, the sheath is composed of crosslinked polymer to maintain some structural integrity during biodegradation. Optionally, the sheath can include bioactive agent. When included, one or more bioactive agents within the sheath can be the same or different from the bioactive agent(s) included in the body of the device.

It will be readily appreciated that the sheath is an optional component. The sheath can be included when it is desirable to contain pieces of the biodegradable polymer as the polymer degrades. In some embodiments, the sheath is configured to allow only pieces of polymer material of a selected size to pass through, and thereby enter the body. These configurations can be particularly desirable, for example, in intraocular applications, where it can be significant to reduce the occurrence of undesirably large particles entering the vitreous, thereby posing risk of interference with vision, damage to eye tissues, and the like. In some aspects, the sheath can function to retain the portions of the biodegradable device after the applicable portions have degraded. In other words, when the implant is fabricated from biodegradable material, the sheath can function to retain portions of the device once the overall integrity of the implant has been reduced to non-functional (for example, when all or substantially all of the bioactive agent has been delivered) pieces of polymeric material. Likewise, when the biodegradable material forms a coating on an implant core, the sheath can function to retain portions of the coating that have separated from the core during the degradation process. Such portions/pieces of the polymeric material can be retained by the sheath unless or until such portions/pieces are reduced to a size that does not pose a risk (for example, a risk of causing vision impairment, ocular tissue damage, and the like) to the patient.

The sheath can be coupled with the implant (for example, utilizing photoreactive groups or thermochemically reactive groups, as described herein). Alternatively, the sheath can be fabricated to encase the implant without being coupled with the implant. According to this latter embodiment, the sheath can form a cladding around the implant and remain associated with the implant by virtue of encasing the implant (as opposed to being chemically coupled to the implant). Put another way, the sheath need not be chemically bonded to the implant according to the invention. According to some aspects of the invention, coupling of the sheath to the polymeric material (such as PEGT/PBT) forming the surface of the implant does not significantly adversely affect biodegradability of the PEGT/PBT polymeric material.

The sheath can be fabricated from a number of materials. In one embodiment, for example, the sheath is fabricated from a matrix of polymeric material such as those described in U.S. Patent Application Publication No. 2003/0129130 A1 (Guire et al., “Particle Immobilized Coatings and Uses Thereof,” Published Jul. 10, 2003).

According to this embodiment, the matrix can be composed of a variety of polymeric material. As used herein, “polymer” and “polymeric material” refer to polymers, copolymers, and combinations and/or blends thereof that can be used to form the matrix. The polymeric material utilized for formation of the matrix can also be referred to as “matrix-forming material,” or “matrix-forming polymeric material.” In some cases the polymeric material is referred to as a “soluble polymer.” Illustrative materials for the matrix of polymeric material include, but are not limited to, synthetic hydrophilic polymers that include polyacrylamide, polymethacrylamide, polyvinylpyrrolidone (PVP), polyacrylic acid, polyethylene glycol, polyvinyl alcohol, poly (HEMA), and the like; synthetic hydrophobic polymers such as polystyrene, polymethylmethacrylate (PMMA), polybutyl methacrylate (PBMA), polyurethanes, and the like; copolymers thereof, or any combination of polymers and copolymers. Natural polymers can also be used and include polysaccharides, for example, polydextrans, glycosaminoglycans, for example hyaluronic acid, and polypeptides, for example, soluble proteins such as albumin and avidin, and combinations of these natural polymers. Combinations of natural and synthetic polymers can also be used.

In one embodiment, the polymers and copolymers as described are derivatized with a reactive group, for example, a latent reactive group such as a thermochemically reactive group or a photoreactive group. The reactive groups can be present at the terminal portions (ends) of the polymeric strand or can be present along the length of the polymer. In one embodiment, the reactive groups are located randomly along the length of the polymer.

The choice of reactive group (for example, the particular type of photoreactive group, or the choice of thermochemically reactive group over photoreactive groups) can depend upon a number of factors. For example, when the invention includes bioactive agent, it can be desirable to utilize thermochemically reactive groups as the reactive group, since many bioactive agents can be susceptible to inactivation during irradiation by light in certain wavelength ranges. Alternatively, inactivation of the bioactive agent can be reduced or avoided by choosing photoreactive groups that are activated by light outside the wavelength range that can affect the bioactive agent. According to these aspects of the invention, inactivation of the bioactive agent means degradation of the bioactive agent sufficient to reduce or eliminate the therapeutic and/or prophylactic effectiveness of the bioactive agent.

In some embodiments, polymer crosslinking compounds, for example photoreactive or thermochemically activated polymer crosslinkers, can be added to the polymeric material and can be treated to form the matrix. As used herein, “polymer crosslinking compound” refers to a compound that can be used to crosslink polymers, copolymers, or combinations thereof, together. The polymer crosslinking compound can include one or more reactive groups, and these groups can be used to crosslink the polymer and/or attach the polymer to the surface of the implant. One example of a useful polymer crosslinking compound is bisacrylamide.

In forming the polymeric matrix, the polymer and a polymer crosslinking compound can be applied to the implant and then treated to crosslink the polymers. The polymer can be crosslinked, for example, by activation of reactive groups provided by the polymer. Addition of polymer crosslinking compounds can serve to make the matrix of polymeric material more durable to use conditions and also can create matrices with controllable pore sizes. The applicability of pore size in the sheath (polymeric matrix material) is described in more detail elsewhere herein.

In some embodiments, the reactive groups provided on the polymer can be photoreactive groups, and the photoreactive polymer can be crosslinked by irradiation. The reactive groups can also serve to bind the polymer to the surface of the implant upon activation of the photoreactive groups.

According to the invention, a “photoreactive polymer” can include one or more “photoreactive groups.” A “photoreactive group” includes one or more reactive moieties that respond to a specific applied external energy source, such as radiation, to undergo active species generation, for example, active species such as nitrene, carbenes and excited ketone states, with resultant covalent bonding to adjacent targeted chemical structure. Examples of such photoreactive groups are described in U.S. Pat. No. 5,002,582 (Guire et al., commonly owned by the assignee of the present invention). Photoreactive groups can be chosen to be responsive to various portions of the electromagnetic spectrum, typically ultraviolet, visible or infrared portions of the spectrum. “Irradiation” refers to the application of electromagnetic radiation to a surface.

Photoreactive aryl ketones are preferred photoreactive groups on the photoreactive polymer, and can be, for example, acetophenone, benzophenone, anthraquinone, anthrone, quinone, and anthrone-like heterocycles (heterocyclic analogs of anthrone such as those having N, O, or S in the 10-position), or their substituted (ring substituted) derivatives. Examples of preferred aryl ketones include heterocyclic derivatives of anthrone, including acridone, xanthone and thioxanthone, and their ring substituted derivatives. Particularly preferred are thioxanthone, and its derivatives, having excitation wavelengths greater than about 360 nm.

The azides are also a suitable class of photoreactive groups on the photoreactive polymer and include arylazides (C₆R₅N₃) such as phenyl azide and particularly 4-fluoro-3-nitrophenyl azide, acyl azides (—CO—N₃) such as ethyl azidoformate, phenyl azidoformate, sulfonyl azides (—SO₂—N₃) such as benzensulfonyl azide, and phosphoryl azides (RO)₂PON₃ such as diphenyl phosphoryl azide and diethyl phosphoryl azide.

Diazo compounds constitute another suitable class of photoreactive groups on the photoreactive polymers and include diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as 3-trifluoromethyl-3-phenyldiazirine, and ketenes (—CH═C═O) such as ketene and diphenylketene. Exemplary photoreactive groups are shown as follows.

TABLE 1

| Photoreactive Group | Bond Formed |
|---|---|
| aryl azides | amine |
| acyl azides | amide |
| azidoformates | carbamate |
| sulfonyl azides | sulfonamide |
| phosphoryl azides | phosphoramide |
| diazoalkanes | new C—C bond |
| diazoketones | new C—C bond and ketone |
| diazoacetates | new C—C bond and ester |
| beta-keto-alpha-diazoacetates | new C—C bond and beta-ketoester |
| aliphatic azo | new C—C bond |
| diazirines | new C—C bond |
| ketenes | new C—C bond |
| photoactivated ketones | new C—C bond and alcohol |

The photoreactive polymer can, in some embodiments, comprise a photoreactive copolymer. The polymer or copolymer can have, for example, a polyacrylamide backbone or be a polyethylene oxide-based polymer or copolymer. One example of a photoreactive polymer comprises a copolymer of vinylpyrrolidone and N-[3-(4-Benzoylbenzamido)propyl]methacrylamide (BBA-APMA); another example is a copolymer of acrylamide and BBA-APMA.

The photoreactive groups of the photoreactive polymer can allow the formation of a covalent bond between the substrate and the photoreactive polymer thereby binding the polymer to the surface of the substrate. The photoreactive groups of the photoreactive polymer can also serve to crosslink polymeric strands together, allowing the formation of a network of covalently crosslinked polymeric strands. When microparticles are included in the polymeric material (as described elsewhere herein), the crosslinked structure can serve as the matrix in which the microparticles can be entrapped. In some embodiments, a non-photoreactive crosslinking agent can be used to promote the formation of crosslinked polymeric strands. The use of a polymer crosslinking agent can depend, for example, on the location and number of photoreactive groups that are present on the polymeric strand. A polymer crosslinking agent can be added that can be a target for the photoreactive groups, that can initiate further polymerization of the polymers, or that can be thermochemically activated crosslinker, for example a DSS(N,N-disuccinimidyl suberate) crosslinker. The crosslinking agents can further solidify the matrix by bonding to other parts of the polymer.

According to some aspects of the invention, the pore size of the polymeric material comprising the sheath can be selected depending upon the application of the inventive implantable device. The pore size should be selected to provide permeability of the sheath to elements required for degradability of the polymeric material of the implant. For example, in embodiments where the implant is fabricated or coated with PEGT/PBT polymer, the sheath should include pores sufficient to allow passage of water through the sheath, thereby permitting hydrolysis of the PEGT/PBT polymeric material. In some embodiments, the pore size can be selected to allow release of elements to the implantation site. In some embodiments, when bioactive agent delivery is also accomplished by the inventive device, the sheath should include pores of sufficient size to allow release of the bioactive agent included in the implant.

In still further embodiments, the sheath can include microparticles that can contain bioactive agent. Preferably, the pore size is sufficient to provide desired features, such as containment of microparticles within the sheath, containment of degradation products, and the like. In other words, the sheath can function to retain microparticles and/or retain degradation particles of microparticles and/or any biodegradable material utilized in association with the device and located within the sheath. For example, the pore size can be selected to permit entrapment of the microparticles within the polymeric matrix material comprising the sheath. For example, if entrapping microparticles with an average diameter of 2.5 μm, it can be useful to have a pore size in the range of 50 nm to 2.5 μm, and more preferably in the range of 100 nm to 1 μm. In any event, one of skill in the art can select a pore size by determining the maximum size of particle (regardless of source of the particle, and thereby including degradation products as well as microparticles themselves) that can be released from the degradable device. In some embodiments, particularly ocular applications of the device, such maximum size can be related to the size of particles believed to be a risk for causing vision impairment, eye tissue damage, and the like.

In one embodiment, the matrix of polymeric material is permeable to various compounds, the compounds typically being smaller than the smallest microparticle immobilized in the matrix. For example, in polymeric matrices that include an insoluble polymeric material, aqueous solutions which can include proteins and other molecules smaller than proteins can diffuse through the matrix.

In one embodiment, a matrix is formed from polymeric material sufficient to entrap the microparticles of the invention and also sufficient to allow the diffusion of molecules in and out of the matrix. In this embodiment, the matrix allows the immobilization of microparticles that are at least 100 nm diameter and allows the diffusion of molecules that are 50 nm or less, and more preferably, 25 nm or less, in and out of the matrix.

Generally speaking, the pore size can be selected depending upon the size of elements to diffuse through the sheath during use. Such passage can be determined by the size of the elements intended to pass through the sheath to reach the device, as well as the size of the elements intended to leave the device and reach the implantation site.

In some aspects of the invention, microparticles can be included in one or more components of the device. According to the invention, microparticles can be provided in the form of microspheres and/or fibers (also referred to herein as “fibrous elements”). The microparticles can be provided with or without bioactive agent. The microparticles can be biodegradable, but this is not required. Microparticles can be included in association with the device to provide one or more features, such as, for example, enhanced imaging of the device, and/or bioactive agent delivery.

In some aspects of the invention, microparticles are included in the sheath. According to these aspects, a mixture is prepared that includes microparticles and polymer material, and the mixture is disposed on the implant and treated to provide the implant with a coating of microparticles immobilized in a matrix of polymer material. In some embodiments, the microparticles are coupled to or associated with one or more functional agents. Such functional agent can be a compound or composition that provides the device with a useful property, such as a biologically, chemically, or physically useful property.

In other aspects, the polymeric material comprising the device (such as an ocular implant) can include microparticles, either alone or in combination with microparticles in the sheath.

The inclusion of microparticles in the sheath and/or the body of the device can provide one or more desirable features to the inventive device and methods. In one aspect, inclusion of microparticles can provide a simple and efficient method for preparing surfaces having diverse properties. For example, inclusion of microparticles can be utilized to provide a surface that can have both biologically useful and detectable properties. In another aspect, the use of microparticles can provide surfaces that are capable of delivering bioactive agent that are not typically compatible in one solvent. In still another aspect, the presence of microparticles in association with the sheath and/or device body can provide a fast and accurate method for preparing surfaces having a precise amount of bioactive agent.

When microparticles are associated with the sheath, a mixture containing a polymeric material and microparticles can be directly disposed on a surface of an implant and then treated to form a polymeric matrix to immobilize the microparticles in the matrix on the surface. Alternatively, the polymeric material can be disposed on an implant and treated, and microparticles can be subsequently disposed on the treated material and immobilized on the implant.

When microparticles are associated with the implant body itself, the microparticles can be included in polymeric material that forms the implant body and/or polymeric material that forms a coating on the surface of the core of the implant. Similar to the embodiment described above, a mixture containing polymeric material and microparticles can be directly disposed on a surface of an implant body and then treated to form a polymeric matrix and thereby immobilize the microparticles in the matrix on the surface. Alternatively, the polymeric material can be disposed on an implant and treated, and microparticles can be subsequently disposed on the treated material and thereby immobilized on the implant body. When the microparticles are incorporated in the implant body itself, the polymeric material can be formed into the implant body (utilizing any of the methods described herein), and the microparticles can be provided in the polymeric material during formation of the implant body.

The microparticles of the invention can comprise any three-dimensional structure that can be immobilized within a polymeric matrix. In some embodiments, the microparticle can also be associated with at least one agent. In these embodiments, the agent or agents associated with the microparticle can impart a desirable property to the surface of the substrate.

According to the invention, the microparticle can be fabricated from any differentially soluble or solid material. Suitable materials include, for example, synthetic polymers such as poly(methylmethacrylate), polystyrene, polyethylene, polypropylene, polyamide, polyester, polyvinylidenedifluoride (PVDF), and the like; degradable polymers such as poly(lactide-co-glycolide) (PLGA) and chitosan (poly-(1,4)-β-D-glucosamine), and the like; glass, including controlled pore glass (CPG) and silica (nonporous glass); metals such as gold, steel, silver, aluminum, silicon, copper, ferric oxide, and the like; natural polymers including cellulose, crosslinked agarose, dextran, and collagen; magnetite, and the like. Examples of useful microparticles are described, for example, in “Microparticle Detection Guide,” from Bangs Laboratories, Fishers, Ind. Optionally, microparticles can be obtained commercially from, for example, Bangs Laboratories (Fishers, Ind.), Polysciences (Germany) Molecular Probes (Eugene, Oreg.), Duke Scientific Corporation (Palo Alto, Calif.), Seradyn Particle Technology (Indianapolis, Ind.), and Dynal Biotech (Oslo, Norway).

In some embodiments, the microparticles are not modified prior to preparation of the microparticle-containing mixture and disposing of the microparticles on the substrate. In these embodiments, the microparticle itself can provide a desirable or useful property when associated with the polymeric matrix on a substrate. For example, paramagnetic microparticles composed of, for example, iron oxide, can provide the surface of a substrate with paramagnetic properties; silica can provide the surface of a substrate with refractive properties; and metallic microparticles can provide the surface of a substrate with reflective properties.

When microparticles are provided in the form of microspheres, they can be provided in any suitable size, but preferably the microsphere is in the range of 5 nm to 100 μm in diameter, or in the range of 100 nm to 20 μm in diameter, or in the range of 400 nm to 20 μm in diameter.

In one embodiment, degradable microparticles can be utilized in association with the sheath. Degradable microparticles can include, for example, dextran, polylactic acid, poly(lactide-co-glycolide), polycaprolactone, polyphosphazene, polymethylidenemalonate, polyorthoesters, polyhydroxybutyrate, polyalkeneanhydrides, polypeptides, polyanhydrides, polyesters, and the like. Degradable polymers useful in the invention can be obtained from, for example, Birmingham Polymers, Inc. (Birmingham, Ala.). Degradable polymers and their synthesis have also been described in various references including Mayer, J. M., and Kapalan, D. L. (1994) Trends in Polymer Science 2:227-235; and Jagur-Grodzinski, J. (1999) Reactive and Functional Polymers: Biomedical Application of Functional Polymers, 39:99-138.

In some cases, the degradable microparticles can be a mixture of a degradable material and a plastic. The degradable material is also preferably nontoxic, although in some cases the microparticles can include an agent that is useful for the selective prevention of prokaryotic or eukaryotic cell growth, or elimination of cells, such as chemotherapeutic agents or antimicrobials. Degradable microparticles can include bioactive agents that can be released from the sheath upon degradation of the microparticle.

In one embodiment, the degradable microparticles can contain a bioactive agent. Degradable microparticles can be prepared incorporating various bioactive agents by established techniques, for example, the solvent evaporation technique (See, for example, Wiehert, B. and Rohdewald, P., J Microencapsul. (1993) 10:195). The bioactive agent can be released from the microparticle, which is immobilized in the polymeric matrix on an implant, upon degradation of the microparticle in vivo. Microparticles having bioactive agent can be formulated to release a desired amount of the bioactive agent over a predetermined period of time. It is understood that factors affecting the release of the bioactive agent and the amount released can be altered by the size of the microparticle, the amount of agent incorporated into the microparticle, the type of degradable material used in fabricating the microparticle, the amount of microparticles immobilized per unit area on the substrate, and the like. The bioactive agent or agents associated with the microparticle can be the same or different from any bioactive agent or agents associated with the polymeric material utilized to fabricate the implant and/or coating on an implant.

In one embodiment, the invention advantageously allows for preparation of surfaces having two, or more than two, different functional agents, wherein the functional agents are mutually incompatible in a particular environment, for example, as hydrophobic and hydrophilic bioactive agents (drugs) are incompatible in either a polar or non-polar solvent. Different functional agents may also demonstrate incompatibility based on protic/aprotic solvents or ionic/non-ionic solvents. For example, the invention allows for the preparation of one set of degradable microparticles containing a hydrophobic drug and the preparation of another set of degradable microparticles containing a hydrophilic drug; the mixing of the two different sets of microparticles into a polymeric material used to form the matrix; and the disposing of the mixture on the surface of a substrate. Both hydrophobic and hydrophilic drugs can be released from the surface of the coated device at the same time, or the composition of the degradable microparticles or polymeric matrix can be altered so that one drug is released at a different rate or time than the other one.

As mentioned herein, the implant body can be fabricated to include the bioactive agent in the body itself, either in addition to, or as a substitute for, bioactive agent included on the surface of the implant. Optionally, a sheath can be provided as well. Use of microparticles in the implant body itself can provide the ability to prepare the device to include otherwise incompatible functional agents, as described above.

In some cases it can be advantageous to prepare degradable microparticles having a composition that is more suitable for either hydrophobic or hydrophilic drugs. For example, useful degradable polymers or degradable copolymers for hydrophobic drugs have a high lactide or high caprolactone content; whereas useful degradable polymers or degradable copolymers for hydrophilic drugs have a high glycolide content.

Traditional coating procedures directed at disposing at least two different types of functional agents have often required that the functional agents be put down onto a substrate separately. In one such example, the coating procedure can involve solubilizing a hydrophobic drug in a non-polar solvent, coating the surface of the substrate with the non-polar mixture, drying the non-polar mixture, solubilizing the hydrophilic drug in a polar solvent, coating the layer of the dried non-polar mixture with the polar mixture, and then drying the polar mixture. This process can be inefficient and can also result in undesirable surface properties (for example, the layering of the drugs can cause one drug to be released before the other one is released). According to the invention, the method of preparing a sheath having two, or more than two, different functional agents, in particular when the two different functional agents are released from the sheath polymeric material, is a significant improvement over traditional methods of coating substrates and delivering functional agents from the surface of the substrates.

Other types of non-degradable microparticles can also be useful for the release of a functional agent from the sheath. Such non-degradable microparticles include pores and can be silica microparticles, for example. Porous non-degradable microparticles can also be used for incorporation of an agent, such as a bioactive agent. Microparticles having particular pore sizes can be chosen based on the type and size of the agent to be incorporated into the pores. Generally, the microparticle having pores can be soaked in a solution containing the desired agent wherein the agent diffuses into the pores of the microparticle. Substrates can be prepared having a coating of these microspheres in a polymeric matrix. Upon placing the coated substrate in fluid-containing environment, for example in a patient, the agent can be released from the microspheres and be delivered to the patient.

The type of polymer, as well as the concentration of the polymer and the extent of polymer crosslinking in the polymeric matrix, can have an affect on the delivery of the bioactive agent from the sheath. For example, polymeric matrix material having charged portions can either decrease or increase the rate of release of a charged bioactive agent from the sheath, depending on whether there are attractive or repulsive forces between the two. Similarly, hydrophilic and hydrophobic polymeric matrix material can also have an affect on the rate of release of hydrophilic and hydrophobic bioactive agents, in particular hydrophilic and hydrophobic drugs. In polymeric matrices having a high concentration of polymer or in matrices wherein the polymer is highly crosslinked, the rate of delivery of the drug can be decreased.

Microparticles can also have an outer coating to control the availability of the agent or agents that are associated with the microparticle. For example, microparticles can include an outer coating of poly(ethylene glycol) (PEG) which can provide sustained or controlled availability of the functional agent that is associated with the microparticle. Another useful outer coating can include, for example, a silane or polysiloxane coating.

In some applications, swellable microparticles can be employed for incorporation of the functional agent. Such swellable microparticles are typically composed of polystyrene or copolymers of polystyrene, and they are typically swellable in an organic solvent. Microparticles can be soaked in organic solvents containing the functional agent to allow incorporation of the agent into the microparticle. The solvent swells the polymeric microparticles and allows the functional agent to penetrate into the microparticles' cores. Excess solvent is then removed, for example, by vacuum filtration, thereby entrapping the functional agent in the hydrophobic interior regions of the microparticles. In one such embodiment, poly(methylstyrene)-divinyl benzene microparticles are rinsed in dimethylformamide. A solution containing the functional agent in dimethylformamide is then added to the microparticles, and the microparticles and solution are incubated with agitation overnight. Excess functional agent is removed from the suspension by vacuum filtration using membrane filters, such as those provided by Millipore Company (Bedford, Mass.). The filtered microparticles are then sonicated and washed by centrifugation in distilled water containing 0.01% Tween 20 to remove residual functional agent on the outside of the microparticles.

In some embodiments it is preferable that the swellable microparticle is impregnated with a functional agent that is detectable using common imaging techniques, for example a paramagnetic material, such as nanoparticular iron oxide, Gd, or Mn, a radioisotope, and non-toxic radio-opaque markers (for example, cage barium sulfate and bismuth trioxide). This can be useful for detection of medical devices that are implanted in the body (that are emplaced at the treatment site) or that travel through a portion of the body (that is, during implantation of the device). Such coated medical devices can be detected by paramagnetic resonance imaging, ultrasonic imaging, or other suitable detection techniques. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep. 1996); other vapor phase chemicals useful for ultrasonic imaging can be found in U.S. Pat. No. 6,261,537 (Issued 17 Jul. 2001).

The microparticles of the invention can possess one or more desirable properties, such as ease of handling, dimensional stability, optical properties, sufficient size and porosity to adequately provide the desired amount of agent or agents to a sheath and/or device body, and the like. The microparticles can be chosen to provide additional desired attributes, such as a satisfactory density, for example, a density greater then water or other solvent used in application of the microparticles to the substrate.

Optionally, the microspheres can include a “coupler” that can allow the coupling of a functional agent to the microparticle. As used herein, the terms “coupler,” “coupling compound,” and “coupling moiety” refer to any sort of entity that allows a functional agent to be attached to the microparticle. The coupler can have one member or more than one member. For example, the coupler can be a small molecule, or can be a binding pair that consists of more than one larger molecule, for example a pair of interacting proteins.

The microparticles can be prepared to include a coupler having reactive groups. The coupler having reactive groups can be used for coupling one or more functional agents to the microparticle, for example, bioactive agents or functional agents conferring optical properties. In other embodiments, reactive groups provided on the microparticle can be used for coupling the microparticle to the polymeric material or for coupling the microparticle to the surface of the substrate, or any combination of the above. Suitable reactive groups can be chosen according to the nature of the functional agent that is to be coupled to the microparticle. Examples of suitable reactive groups include, but are not limited to, carboxylic acids, sulfonic acids, phosphoric acids, phosphonic acids, aldehyde groups, amine groups, thiol groups, thiol-reactive groups, epoxide groups, and the like. For example, carboxylate-modified microparticles can be used for covalent coupling of proteins and other amine-containing molecules using water-soluble carbodiimide reagents. Aldehyde-modified microparticles can be used to couple the microparticles to proteins and other amines under mild conditions. Amine-modified microparticles can be used to couple the microparticle to a variety of amine-reactive moieties, such as succinimidyl esters and isothiocyanates of haptens and drugs, or carboxylic acids of proteins. In another application, sulfate-modified microparticles can be used for passive absorption of a protein such as bovine serum albumin (BSA), IgG, avidin, streptavidin, and the like.

In another embodiment, the reactive groups can include such binding groups as biotin, avidin, streptavidin, protein A, and the like. These and other modified microparticles are commercially available from a number of commercial sources, including Molecular Probes, Inc. (Eugene, Oreg.).

Another method for coupling moieties of the invention is through a combination of chemical and affinity interactions, herein referred to as “chemi-affinity” interactions, as described by Chumura et al. (2001) Proc. Natl. Acad. Sci., 98:8480. Binding pairs can be engineered that have high binding specificity and a negligible dissociation constant by functionalizing each member of the binding pair, near the affinity binding sites of the pair, with groups that will react to form a covalent bond. For example, the constituents of each functionalized member can react, for example by Michael addition or nucleophilic substitution, to form a covalent bond, for example a thioether bond.

The surface of the microparticle can also be coated with crosslinking compounds. Various functional agents can be coupled to the microparticle via crosslinking agents. Commercially available crosslinking agents obtained from, for example, Pierce Chemical Company (Rockford, Ill.) can be used to couple the microparticles to functional agents via, for example, amine groups, provided on the surface of the microparticles. Useful crosslinking compounds include homobifunctional and heterobifunctional crosslinkers. Two examples of crosslinking compounds that can be used on microparticles presenting, for example, amine groups, are di-succinimidyl suberate and 1,4-bis-maleimidobutane.

In some embodiments, the microparticles are associated with a functional agent. As used herein, a “functional agent” refers to a compound that can be coupled to, or associated with, the microparticles to provide the surface of the coated substrate with a property that is conferred by that compound. Useful functional agents include bioactive agents, compounds with detectable properties, such as paramagnetic compounds, and compounds with optical properties. The microparticles of the invention can be coupled to, or associated with, any physiologically active substance that produces a local or systemic effect. For ease of discussion, reference will repeatedly be made to a “functional agent.” While reference will be made to a “functional agent,” it will be understood that the invention can provide any number of functional agents to a treatment site. Thus, reference to the singular form of “functional agent” is intended to encompass the plural form as well.

The quantity of functional agents associated with each individual microparticle can be adjusted by the user to achieve the desired effect. The density of functional agents coupled to, or associated with, the microparticles can vary and can depend upon, for example, the dose of a particular bioactive agent intended to be provided on the sheath. Bioactive agents can be provided by the microparticles in a range suitable for the application. In another example, protein molecules can be provided by microparticles. For example, the amount of protein molecules present can be in the range of 1-250,000 molecules per 1 μm diameter microparticle. However, depending on microparticle source and preparation the amount of agent coupled to, or associated with, the microsphere can vary.

The quantity and organization of the microparticles themselves within or on a sheath can also impart desirable properties to the implant, for example, in imagining the device within the patient's body. For paramagnetic resonance or ultrasonic imaging applications, the number of microparticles associated with a device can be directly correlated with the imaging signal strength. To increase imaging signal strength, a high density of microparticles can be immobilized in a localized area on the device. Alternately, the density of microparticles over the device can vary, thereby allowing different regions of the device to be imaged distinctly. This can be accomplished by coating the different regions of the device with two or more different coating slurries with differing concentrations of microparticles.

Coupling the functional agent to, or associating the functional agent with the microparticle prior to disposing the microparticle on the sheath can provide benefits. It is understood that the functional agent can be provided within or on the surface of microparticles. For example, as compared to directly coupling an agent to a substrate, a higher density of agent per surface area of substrate can be achieved by first loading the functional agent on or in the microparticle. Also, coupling of an agent to the microparticle in solution is generally more efficient than the direct coupling of a functional agent to a substrate, resulting in a lower loss of functional agent during the coupling procedure. Additionally, coupling of a functional agent to a microparticle in solution generally allows for more variability during the coupling process. For example, coupling procedures that require agitation of the coupling solution, such as stirring, can readily be achieved using microparticles in the stirred solution. Additionally, determination of the amount of functional agent coupled per microparticle can readily be achieved by performing, for example, immunofluorescence flow cytometry or a protein assay, such as a BCA assay, on a portion of the microparticles following coupling to the functional agent. Once the microparticles have been coupled with the desired amount and type of functional agent, these functional agent-coupled microparticles can then be included in a mixture containing a suitable polymeric material or can be disposed on a substrate that has been coated with a polymeric material.

In some embodiments, the functional agent can be modified prior to coupling with the microparticle. In other words, a portion of the coupler can be attached to the functional agent prior to the functional agent being coupled to the microparticle. For example, the functional agent can be derivatized with one member of a binding pair, and the microparticles derivatized with the other member of the binding pair. Suitable binding pairs include avidin:biotin, streptavidin:biotin, antibody:hapten, for example anti-digoxigenin Ab:digoxigenin or anti-trinitrophenyl Ab:trinitrophenyl. For example, the functional agent can be biotinylated by, for example, cross-linking the biotin to the functional agent using methods known in the art. The biotinylated agent or agents can then be coupled with streptavidin provided on the surface of the microparticles. Members of the binding pair can be functionalized to provide chemi-affinity interactions as indicated elsewhere herein.

As described herein, the microparticles can be immobilized in the polymeric matrix forming the sheath by entrapment of the microparticles. In another embodiment, immobilization of the microparticles can be performed by chemical bonding of the microparticle to the matrix and the matrix to the substrate. A variety of bonds can be formed between the microparticles and the matrix material, and the matrix material and the substrate. These bonds include, for example, ionic, covalent, coordinative, hydrogen and Van der Waals bonds. For example, it can be desirable to maintain the microparticles within the sheath (as opposed to releasing the particles and/or allowing the microparticles to degrade over time within the patient). This can occur, for example, when the microparticles are utilized for imaging the device within the patient, or when bioactive agent is provided on the surface of the sheath and it is desired to maintain the bioactive agent surface on the implant while the implant is in the patient.

In one embodiment, slurries including polymeric material and microparticles, which can be coupled to, or associated with, a functional agent, are dip-coated onto the surface of the implant to form a coated surface (sheath). In another embodiment the polymeric material is dip-coated to form a coated surface (sheath). Alternatively, the polymeric material can be applied by jet printing to the surface of the substrate through utilization of a piezoelectric pump. Printing techniques can allow the application of a relatively small amount of the mixture at precise locations on the surface of the substrate. In another embodiment, the polymeric material is disposed on the substrate and treated; the microparticles are then placed and immobilized on the substrate via the treated material.

In some embodiments, the thickness of the matrix of polymeric material forming the sheath is greater than the diameter of the largest microparticle being associated with the sheath. However, providing a matrix having a thickness greater than the diameter of the largest microparticle is not required, and microparticles can be immobilized without completely entrapping the microparticle within the matrix material. In some applications, the implant can be subject to more than one step of coating with a mixture of polymeric material and microparticles and treating, thereby allowing the formation of a sheath composed of multiple layers.

Use

In use, the implantable device is placed within a patient at a desired implantation site. Upon contact with body fluids, the body fluids initially permeate at least a portion of the biodegradable composition, allowing for dissolution and diffusion of the bioactive agent from the biodegradable composition. The biodegradable composition undergoes gradual degradation (usually primarily through hydrolysis) with concomitant release of the dispersed bioactive agent for a sustained or extended period. This can result in prolonged delivery of therapeutically and/or prophylactically effective amounts of the bioactive agent.

In some embodiments, the implants are inserted directly into the eye “as is.” In other embodiments, implant insertion devices (for example, a tube-like device in which the implant is loaded and inserted into the eye) may be used to facilitate insertion of the implant into the subretinal space. Such insertion devices can eliminate the need to use micro-forceps and similar devices to load and position the implant within the eye.

In some aspects, the invention features methods for the treatment and prevention of disorders and or diseases of the eye, in particular retinal/choroidal disorders or diseases, by administering to a desired treatment site, particularly the choroid and the retina, one or more bioactive agents. In particular, the methods provide administering one or more bioactive agents to a treatment site by implanting the bioactive agents within the eye. In one embodiment, an implant of the invention is inserted within the eye to provide sustained delivery of the bioactive agent to the desired treatment site. Such methods provide localized, sustained delivery of the bioactive agent subretinally at the treatment site without major trauma or the need for fluid dissection of the retina.

In one illustrative method, the implant is located in one or more tissue layers above the choroid below the nerve fiber layer. Further, if desired, two or more implants may be simultaneously implanted, potentially encircling the disease site. Further, it is desired that the implant acts both as a sustained release drug delivery system and as a body that is capable of self-piercing and/or penetrating the eye structure to achieve implantation within the eye. However, it is also possible to provide an incision in the eye through which the implant is inserted.

One method for inserting the implant involves performing a standard pars plana vitrectomy, and inserting the implant into the subretinal space through the pars plana vitrectomy. In particular, the subjects are first anesthetized, for example, with an intramuscular injection of ketamine hydrochloride and xylazine hydrochloride. Next the pupils are dilated with phenylephrine and tropicamide. A peritomy is then made at the superotemporal and superonasal quadrants. An infusion pipe line may be inserted through the superonasal sclerotomy and a vitreous cutter inserted through the superotemporal sclerotomy. The vitreous cutter and infusion pipe may then be used to perform a 2-port core vitrectomy. The illumination provided by an operating microscope is sufficient for the operation. Using intraocular microscopic forceps, the implant is inserted in the subretinal space through a small self-starting retinotomy. The implant may have a bevel shaped tip, thereby facilitating insertion into the subretinal space. The implant is left in position and the forceps withdrawn from the eye. No laser retinopexy need be applied to seal the retinal break. The infusion line is removed and the sclerotomies and conjuctival openings closed.

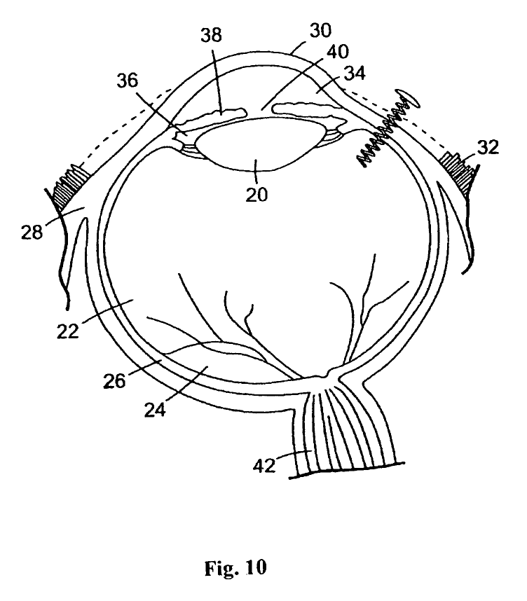

For intraocular implants, the following procedure can be applicable. A sclerotomy can be created for insertion of the controlled delivery device into the posterior portion of the eye. Conventional techniques can be used for the creation of the sclerotomy. Such techniques include the dissection of the conjunctiva 32 and the creation of pars plana scleral incisions through the sclera 28. As shown in FIG. 10, the dissection of the conjunctiva 32 typically involves pulling back the conjunctiva 32 about the eye so as to expose large areas of the sclera 28, and the clipping or securing of the conjunctiva 32 in that pulled back state (the normal position of the conjunctiva is shown in phantom). In other words, the sclera 28 is exposed only in the areas where the pars plana scleral incisions are to be made. Surgical instruments used in the procedure are then passed through these incisions. Thus, the incisions should be made large enough to accommodate the instruments required for the procedure.

Alternatively, the creation of the sclerotomy can be accomplished by use of an alignment device and method, such as that described in U.S. patent application Ser. No. 09/523,767, that enables sutureless surgical methods and devices thereof. In particular, such methods and devices do not require the use of sutures to seal the openings through which instruments are inserted. The alignment devices are inserted through the conjunctiva and sclera to form one or more entry apertures. Preferably, the alignment devices are metal or polyimide cannulas through which the surgical instruments used in the procedure are inserted into the eye.

In further embodiments, the device can be implanted directly through a self-starting transconjunctival trans-scleral “needle stick.” For example, the body member 2 of the device can include a sharp tip 10, such as that illustrated in FIG. 8. According to this embodiment, the sharp tip 10 can be utilized to pierce the body and thereby create the incision site and access to the implantation site. In this case, no conjunctival surgery or extraneous alignment device is necessary.

In further embodiments, the conjunctival tissue can be dissected to expose a portion of the pars plana region, and a needlestick can be made into the sclera in the exposed region. A self-starting coil that includes a sharp tip is then inserted through the pars plana at the site of the needlestick, and the coil is rotated through the sclera until the cap of the device abuts the sclera. In some preferred embodiments, the needlestick is smaller than the diameter of the body member of the implantable device (for example, a 30-gauge needlestick can be used with an implantable device having a body member with a diameter of 0.5 mm or less). The conjunctival tissue is then pulled over the cap, to provide a flap or “seal” over the device, thus minimizing irritation of the implantation site, foreign body sensation, and the like. Optionally, the conjunctival tissue can be further secured by a single suture (in preferred embodiments, a biodegradable suture).

In some embodiments, it can be preferable to create an incision site that is slightly larger than the dimensions of the proximal portion of the body member. For example, when the device includes a cap 8 and is implanted into the eye, it can be preferable to create an incision that is larger than the largest diameter of the cap 8, such that the cap sits below the outer surface of the sclera. For example, a partial incision in the sclera can be made to create a scleral flap. Once the device has been implanted, and the cap 8 is placed so that it abuts the incision site, the scleral flap can be folded back over the device, thus providing a covering over the cap. Alternatively, when the proximal end of the body member does not include a cap 8, a flap-like cover can still be utilized to cover the proximal end of the device, in accordance with the description above. Preferably, these embodiments minimize the contact of the proximal end (for example, the cap 8) of the device with other body tissues, thereby reducing such risks as irritation of body tissues, and/or translation of movement of the eye to the device, thereby potentially damaging eye tissues. This can provide one or more advantages, such as reduced tendency for movement of the eye to be translated to the controlled delivery device, since the proximal end of the device will not be sitting at the surface of the eye and thus in contact with other body tissues; and reduced irritation of surrounding tissues.

The body member 2 is then inserted into the eye. For example, in embodiments wherein the body member 2 has a coil shape, the body member 2 is inserted into the eye by rotating or twisting the body member 2 into the eye until the cap 8 abuts the outer surface of the eye. In embodiments wherein the body member 2 is fabricated of a shape memory material, the shape memory material is first cooled to a temperature at which the martensite phase is stable and the device is deformed, for example, into a linear shape. The device is then inserted into the eye. To return the device to its memory shape, the device is left unrestrained and is simply allowed to reach a temperature (for example, by heating the device) above the martensite phase temperature. For example, the shape memory material can be heated by a laser to return the device to a temperature above the martensite phase temperature. The shape memory material can also be selected such that the martensite phase temperature is below body temperature so that the material is simply cooled to below body temperature, deformed to a linear shape, and inserted into the eye. Then, as the material warms up within the eye to body temperature, the device can return to its remembered shape. As discussed herein, when laser application is utilized, conditions are preferably controlled to maintain such parameters as wavelength and temperature, to minimize adverse effect on the polymeric coated composition.

FIG. 10 illustrates a controlled delivery device according to one embodiment of the invention that is implanted in the eye. When implanted into the eye, it is desirable to limit the length L of controlled delivery devices to prevent the controlled delivery device from entering the central visual field. If the implant enters the central visual field, this can result in blind spots in the patient's vision and can increase the risk of damage to the retinal tissue and lens capsule. Thus, for example, when the controlled delivery device is inserted at the pars plana (as shown in FIG. 10), the distance from the implantation site on the pars plana to the central visual field is preferably less than about 1 cm.

Optionally, after the device is implanted into the eye, the cap 8 can then be sutured or otherwise secured to the sclera to maintain the controlled delivery device in place. In preferred embodiments, no further manipulation of the device is required for delivery of one or more bioactive agents to the interior of the eye. The conjunctiva can be adjusted to cover the cap 8 of the device, when desired, and the surgical procedure is completed.

In other embodiments, when a lumen is included in the device for delivery of one or more additional substances to the interior of the eye, further steps can be included as follows. If a cover is used to close the port(s), it is removed at this time, and if used, a collar for providing a snug fit about the injection mechanism (such as a syringe) is provided. The injection mechanism is then connected with the port(s) for injection of one or more substances to the controlled delivery device. If the port(s) are composed of a self-sealing material through which the needle of an injection mechanism can be inserted and which seals off automatically when the injection mechanism is removed, the injection mechanism is simply inserted through the port and the substance injected. Following injection, the conjunctiva can be adjusted to cover the cap 8 of the device, if desired.

In some embodiments, the core can be provided in the form of one or more fibrous elements. Optionally, the fibrous element can comprise a non-biodegradable element of the overall device. Alternatively, the fibrous element can comprise a biodegradable element of the device. In still further embodiments, the fibrous element can be selected and formulated to degrade at a different rate than other elements of the overall medical device. Generally, fibrous elements (whether biodegradable or not) can be desired, for example, to provide additional structural support to the device, and/or to provide a suitable surface area for delivery of bioactive agent without exceeding implantation site constraints. For example, fibers can be particularly useful for subretinal implants in accordance with the invention. The choice of biodegradable or non-degradable material to fabricate the fibrous element can depend upon the application of the device, and whether the user desires to maintain the fibrous elements within the patient's body after other portions of the device degrade. The fibrous elements can be embedded within the degradable polymeric material.

In one such embodiment, non-biodegradable fibers are included in the degradable polymeric material used to make an implant for subretinal delivery of bioactive agent. According to this embodiment, the polymeric material comprising the polymer matrix will degrade over time, leaving the non-biodegradable fibers at the implantation site. The fibers can provide structural integrity of the implant at the implantation site, and during residence of the medical device

Fibrous elements can be included within the degradable polymer matrix in a number of ways. In one embodiment, fibers are added to a mixture of dimethylterephthalate, butanediol (in excess), polyethylene glycol, an antioxidant, and catalyst. The reaction mixture is then subject to a synthesis procedure described elsewhere herein (the particular synthesis procedure will depend, of course, upon the polymeric material; for example, when the polymeric material comprises PEGT/PBT, the synthesis generally includes steps of transesterification, distillation of excess butanediol, and condensation of a prepolymer of butanediol terephthalate with the polyethylene glycol to form a PEGT/PBT copolymer). In an alternative embodiment, a polymer (such as a PEGT/PBT copolymer) can be formed and subsequently subjected to temperatures sufficient to “melt” the polymer. According to this embodiment, the polymer will achieve a temperature sufficient to allow fibers to be mixed within the polymer melt, but not sufficient to alter the properties of the polymer for its intended use. After the fibers are mixed with the polymer melt, the melt can be permitted to form a solid polymer material through evaporation of solvent or through cooling of the melt.

In yet a further embodiment, the fibers can be combined with a reactive polymer, followed by polymerization to form a polymer matrix that includes the fibrous material. For example, polymer matrix structures can be formulated by mixing selected monomeric components with polymerization facilitating compounds, such as one or more initiators and/or activators. One illustrative polymeric composition has been formulated by I. Chung et al. (European Polymer Journal 39:1817-1822 (2003)). Chung et al. formulated network structures by thoroughly mixing selected oligomers with a photoinitiator and an activator. More specifically, polycaprolactone trimethyacrylate (PCLTMA) and di(propylene fumarate)-dimethacrylate (DPFDMA) were mixed with DL-camphoroquinone (CQ, 0.7 weight %, a photoinitiator) and 2-(dimethylamino)ethyl methacrylate (DMAEM, 1.4 weight %, an activator). The mixture was then exposed to blue light source for ten minutes at room temperature. The cured specimens were then removed from molds and conditions in PBS solution. By modifying the formulation of the polymeric compositions, such features as degradation rates, strength, viscosity were controllable. Thus, such compositions could be utilized in the inventive methods and devices as well. Fibrous elements can be combined with the monomeric components and polymerization facilitating compounds and polymerized to form polymeric network structures that include fibrous elements. Other reactive polymers are known and can be readily adapted for use with the inventive concepts described herein. These coated fibers can then be mixed with the degradable polymers described herein.

Preparation methods for fibrous polymer materials are described, for example, in U.S. Pat. No. 6,685,957 (Bezemer et al., “Preparation of Fibrous Polymer Implant Containing Bioactive Agents Using Wet Spinning Technique”) and U.S. Patent Publication No. U.S. 2004/0086544 (Bezemer et al., “Polymers with Bioactive Agents”). According to these particular embodiments, a wet spinning technique is utilized to provide polymer loaded with one or more bioactive agents. Preparation of one such copolymer will be explained by way of example for a PEGT/PBT copolymer. Utilizing the teaching herein, the skilled artisan will be able to prepare any number of copolymers that include bioactive agent.

A PEGT/PBT copolymer can be synthesized as described above (transesterification, followed by distillation, and condensation). The bioactive agent to be loaded into the polymer can be chosen from any suitable bioactive agent. Some exemplary bioactive agents are mentioned herein. Generally, the bioactive agent-loaded polymer is formed by preparing an aqueous solution of the bioactive agent, and adding the bioactive agent solution to a solution of amphiphilic block copolymer containing hydrophobic blocks dissolved in a first solvent that is immiscible with water to form an emulsion. The emulsion is injected through a nozzle into a second solvent that is miscible with the first solvent and in which the copolymer is essentially insoluble. The result after injection is a solid copolymer fiber loaded with the bioactive agent. The fiber can then be shaped into an implant, if desired. Typically, for preparation of the water-in-oil emulsion according to these embodiments, it is desired that a hydrophobic bioactive agent dissolves at least slightly in water, preferably at least to such an extent that the resultant loaded polymer comprises an amount of the bioactive agent sufficient to achieved a desired effect in vivo. Optionally, a surfactant can be added to the aqueous solution of the bioactive agent in order to allow a minimal desired amount of the bioactive agent. Examples of such surfactants are well known to the skilled artisan and can be used in amounts that can easily be optimized by the artisan. Specific examples of suitable surfactants include, but are not limited to, poly(vinyl) alcohol, Span 80, Tween, and Pluronic.

According to these embodiments of the invention, two solvents are chosen to complement each other's action in the synthesis process. The first solvent is chosen to be immiscible with water. In addition, the polymer that is to be loaded with bioactive agent should be soluble in the first solvent. The second solvent is chosen such that the polymer is insoluble therein. Also, the first solvent is selected to be well miscible with the second solvent. Preferably, the first solvent mixes better with the second solvent than the polymer dissolves in the first solvent. This helps ensure that, upon immersion of the water-in-oil emulsion in the second solvent, the first solvent will substantially completely migrate into the second solvent. Preferably, both the first and second solvents are immiscible with water. This makes it possible to prevent contact between the bioactive agent, which is processed in an aqueous solution, with an organic solvent, which can be harmful to the bioactive agent. Depending upon the nature of the polymeric material to be loaded, the skilled person can readily select suitable solvents utilizing the teaching herein. By way of example, when the polymer is PEGT/PBT copolymer, a suitable first solvent is chloroform, and a suitable second solvent is hexane.

In a first step of the process, a solution is provided of the polymer in the first solvent. The concentration of this solution is not critical and can be determined based upon such factors as the amount of solvent sufficient to dissolve all of the polymer, and overall efficiency of the process.

A water-in-oil solution is prepared by mixing the polymer solution with an aqueous solution of the bioactive agent. Under certain circumstances, it can be desired to add conventional stabilizers to enhance the stability of the water-in-oil emulsion. Typical examples of such stabilizers include proteins such as albumin or casein, Pluronic, and Span 80. Such stabilizers are optional only.

According to these embodiments, the amount of bioactive agent in the aqueous solution can be chosen such that a desired amount of the bioactive agent is eventually incorporated into the polymer. The amount of bioactive agent incorporated in the polymer can depend upon such factors as the type of polymer and the nature of the bioactive agent. In the case of proteins and peptides, for example, at least 0.01 weight percent (based upon the weight of the loaded polymer) of the protein or peptide will be incorporated. For proteins and peptides, up to about 10 weight percent (based upon the weight of the loaded polymer) can be incorporated into the polymer. When using particularly hydrophilic bioactive agents, the agent can be incorporated in a concentration of up to 50 weight percent (based upon the weight of the loaded polymer).

The amount of water used for preparing the aqueous bioactive agent solution will be sufficiently high to enable an efficient dissolution of the bioactive agent without employing unduly harsh conditions that might adversely affect the stability and/or biological activity of the bioactive agent. The upper limit of the amount of water used can depend upon the rate at which the bioactive agent is to be released from the polymer in a final application. The use of larger amounts of water typically leads to higher release rates of the polymer. Typically, the aqueous solution of the bioactive agent will comprise about 0.001 to about 10 weight percent of bioactive agent, based upon the weight of the solution. In practice, the amount of bioactive agent in the solution will depend upon the solubility of the bioactive agent or agents chosen, and on the stability of the water-in-oil emulsion.

The obtained water-in-oil emulsion is next immersed in the second solvent by injection through a nozzle. The diameter and shape of the nozzle can be varied to obtain fibers of different diameter and shape. The injection itself will typically be driven by a pressure that transports the emulsion through the nozzle into the second solvent. For example, injection can be accomplished by use of a syringe or an extruder. The amount of the second solvent is not critical and can be selected to be at least sufficient for the emulsion to be completely immersed in it and to allow a substantially complete migration of the first solvent from the emulsion into the second solvent. The upper limit will generally be chosen on the basis of economic considerations.

Upon immersion of the emulsion into the second solvent, the first solvent will migrate from the emulsion into the second solvent due to the specific selection of the first and second solvents. In practice, it can often be observed that first exchange of the first and second solvents takes place before the first solvent will migrate into the second solvent. This results in polymer fibers provided with a porosity. P. van de Witte (“Polylactide membranes. Correlation between phase transitions and morphology,” PhD thesis, University of Twente, Enschede, 1994) describes this phenomenon and how it can be controlled to obtain a desired porosity.

As a result, the polymer, which does not dissolve in the second solvent, will solidify and thereby incorporate the bioactive agent. Finally, the solid loaded polymer can be removed from the mixture of the first and second solvents in any convention manner and can eventually be dried.

In some embodiments, the obtained fibers can be formed into an implant having desired dimensions by collecting the fibers in a mold, and bonding them together (for example, by use of a suitable solvent mixture). According to these embodiments, the mixture should comprise at least one solvent in which the polymer does not dissolve. Preferably, a mixture is used of the above described first and second solvents. The second solvent will typically be present in an amount exceeding that of the first solvent, in order to reduce the risk of any of the polymer dissolving in the solvent mixture. Preferably, the volumetric ratio of the first solvent to the second solvent is in the range of 1:1 to 1:3.

Other methods of synthesizing a polymer containing fibers are known and will not be discussed in detail herein.

In another aspect, fibers composed of polyethylene (PE) can be desirable for use in composite materials use in biomedical devices. PE fibers exhibit high strength, chemically stability, low density, and biocompatibility. However, use of PE fibers in composites has been limited largely by their surface properties, which can hinder adhesion. Thus, surface modification of such fibers can provide an improved composite material that includes the fibers.

In some embodiments, it can be preferable to modify the surface of the fibers (degradable or non-degradable). Most polymer blends are immiscible, and thus, the components of a polymer blend phase often separate into distinct, macroscopic domains. These macroscopic domains can be undesirable in a composite material, since they can lead to voids within the polymer composite material, as well as instability in the polymer blend as a result of nonhomogeneity of the polymer components.

In order to provide effective reinforcement, there should exist good stress transfer at the interface of the fiber and polymer material with which the fiber is associated. The stress transfer at the interface between two different phases in the solid state is determined by the degree of adhesion. Adhesion to fibers can be limited by their surface morphology, chemical inertness and/or low surface energy. Thus, strong chemical or physical bonding between the two materials can be important to achieve adhesion. The chemical bonding can be described by ionic, covalent, or metal bonds, whereas the physical bonding is represented by London dispersion forces, van der Waals forces, hydrogen-bonding, polar-polar bonds, and the like.

In some aspects of the invention, surface modification of fibers is achieved by chemically roughening the surface of the fiber to minimize the size of any surface defects. According to these aspects, surface roughening can be accomplished by either degrading the outer layer of the fiber or building it up by a grafting process. Methods to improve adhesion of fibers can include reactive plasmas, irradiation, chemical etching, and ozonolysis. These methods are discussed, for example, in Brennan, A. B., “Surface Modification of Polyethylene Fibers for Enhanced Performance in Composites,” Trends in Polymer Science, (1995), vol. 3:12-21.

In some aspects, surface modification is accomplished by plasma treatment, which involves a complex series of reactions with free radicals, cations, electrons, and the excited states created by the excitation of a gas at either a reduced pressure or ambient pressure. The effect of the plasma on the surface can be described in general terms as either polymer-forming or non-polymer-forming (also referred to as ablative) reactions. Polymer-forming reactions are induced by plasmas formed from most organic gases. The polymers formed by these reactions typically have reactive functional groups that enhance the formation of both chemical and physical bonds with adherents. The non-polymer-forming plasmas include those from oxygen, nitrogen, hydrogen, argon, and ammonia. The action of these plasmas involves abstraction of protons and creation of unstable radicals that, upon exposure to oxygen, convert to functional groups such as alcohols, aldehydes, ketones, and carboxylic acids. The ablative process involves removal of the outer portion (typically 5 to 50 nm) of the fiber.

Surface modification can also be accomplished by ionizing radiation from a gamma source such as ⁶⁰Co. In the presence of reactive organic monomers, ionizing radiation can create polymeric grafts on the surface of the fiber. Gamma radiation penetrates into the bulk of the fiber material and produces cations, cation radicals, free radicals, and other reactive intermediates. One illustrative example will be described. Poly(cyclohexyl methacrylate) (PCHMA), poly(N-vinylpyrrolidone (PVP) and poly(n-butyl acrylate) (PBA) can be grafted onto the surface of fibers using ⁶⁰Co gamma radication. Typically, PE will undergo crosslinking and chain-scission reactions when exposed to high doses of gamma radiation; thus, low dosages and dose rates can be preferred in some applications.

Surface modification of fibers by irradiation with an electron beam is another method that can be utilized.

In still further embodiments, wet chemical methods can be utilized to provide surface modification of the fiber. In contrast to the methods described above, these methods are chemical processes performed in the absence of any external radiation. Wet chemical methods typically involve strong oxidizing agents. For example, PE fibers can be coated by mixing in a solution of poly(hydroxyethyl methacrylate) (PHEMA) and dimethylformamide. The fibers can be allowed to swell in benzoyl peroxide (BPO) at 50° C. Each fiber can then be incorporated into a selected polymer mixture (including any of the polymer materials described herein) that is subsequently moulded and reacted to form a composite.

Thus, to enhance the structural integrity and mechanical properties of a polymeric material associated with fibers, copolymer “compatibilizers” can be added to the polymer mixture. In preferred embodiments, compatibilizers effectively act as high molecular weight surfactants, in that they can localize at the interface between the immiscible polymers, interlink the phase-separated regions of the polymer blend, lower the interfacial tension, and disperse the incompatible polymers into smaller domains. Consequently, the degree of adhesion between the phase-separated regions and the mechanical properties of the material can be significantly enhanced.

One illustrative example of suitable compatibilizers includes graft copolymers. Graft copolymers contain a backbone and side chains that emanate from the backbone. The side chains of the graft copolymer intertwine across the polymer-polymer interface and effectively bind the two phase-separated regions. Gersappe, D. et al. ((1994) Science 265:1072-1074) describe suitable graft copolymers for use as compatibilizers, as well as methods to determine suitable graft copolymers for such use. For example, a four-component blend composed of two immiscible, phase-separated homopolymers, A and B, and two types of graft copolymers, AC and BD can be designed as compatibilizers. The backbones of the AC copolymers are formed entirely from A segments, whereas the side chains are formed from C units. Similarly, for the BD chains, the backbones are formed entirely of B segments, while the D segments are the side chains. Generally speaking, the A and B backbones of the compatibilizers are formed from incompatible polymers, while the C and D side chains are formed from highly compatible polymers. The high interfacial tension between the immiscible homopolymers drives the grafts to the A-B boundary. The compatibilizers can then localize at the interface, with the C and D side chains intertwining across the A-B layer. The side chains thread through and bind across the interface. Exemplary A and B homopolymers include poly(ethyl acrylate) (PEA) and poly(methyl methacrylate) (PMMA). The side chains C and D were polystyrene (PS).

Suitable fibers include fibrous materials of sufficient strength to provide the desired properties to the inventive device. For example, nanofibers are commercially available and can be utilized in accordance with the teachings herein. In embodiments where the nanofibers remain at the implantation site after degradation of the polymer, fibers with nanometer to micro diameter can be preferred.

Optionally, the fibers can be fabricated to include one or more bioactive agents, either in addition to, or instead of, other portions of the device. Use of bioactive agent in association with the nanofibers can provide multiple bioactive agents and/or the same drug with multiple release rates to be used in connection with the same device.

The fibers can be fabricated to include bioactive agent in any suitable manner. In one embodiment, viscous polymer solutions containing bioactive agent can be forced through a small orifice into a solvent that does not dissolve the bioactive agent or the polymer material, thus creating filaments. The diameter of the filaments can be dependent upon the orifice diameter.

As discussed herein, the biodegradable polymer material can be selected and formulated to provide a desired controlled release of bioactive agent to a treatment site. As described herein, controlled release at the treatment site can mean control both in dosage rate and total dosage. In some embodiments, the configuration of the device can be manipulated to control release of the bioactive agent. For example, the surface area and/or size of the device can be manipulated to control dosage of the bioactive agent(s) provided to the implantation site. In other aspects, incorporation of the bioactive agent in microspheres, fibers, or other delivery devices, can impact release rate of the bioactive agent, as will be apparent from the discussion herein. Further, as described herein, the composition of the polymeric matrix can itself be manipulated to affect release rate of the bioactive agent.

In preferred aspects, the inventive biodegradable compositions can provide a controlled release of bioactive agent to thereby provide a therapeutically effective dose of the bioactive agent for a sufficient time to provide the intended benefits. The controlled release includes both an initial release and subsequent sustained-release of the bioactive agent.

The inventive devices provide release of bioactive agent over time, and this relationship can be plotted to establish a release profile (cumulative mass of bioactive agent released versus time). Typically, the bioactive agent release profile can be considered to include an initial release of the bioactive agent, and a release of the bioactive agent over time, and the distinction between these two can often be simply the amount of time. The initial release is that amount of bioactive agent released shortly after the device is implanted, and the release of bioactive agent over time includes a longer period of time (for example, the lifespan of the biodegradable composition).

In some cases, the initial release can be characterized as a “burst” release. For systems that provide a “burst release” of bioactive agent, an initial release of a significant amount of bioactive agent is observed within a relatively short period of time after an implantable device is provided within a patient. A typical burst release is a much higher release in a relatively short amount of time (for example, more than 30% of the amount of bioactive agent contained in the coating within the first 24 hours after implantation). In contrast, coatings can provide substantially linear release of bioactive agent, wherein the initial release of bioactive agent does not comprise a significantly different slope or shape than the overall release profile. Put another way, a burst release can be characterized as an initial release that differs in magnitude of bioactive agent released (that is, a significant amount is released during the initial period).

The significance of a burst release can also be considered in relation to the particular polymeric material that contains the bioactive agent. For example, for a biodegradable polymer having a half-weight degradation time of four weeks, a significant burst release can be considered to be more than about 30% of the bioactive agent contained in the coating that is released within the first 24-hour period. For a biodegradable polymer having a half-weight degradation time of more than four weeks, a longer burst time period can be considered significant for the same amount of bioactive agent. For example, the half-weight degradation time of PLA is 155 days compared to 30 days for PLGA. Thus, a longer time period would be considered therapeutically relevant for the burst release from PLA compared to PLGA.

In designing an implantable, biodegradable medical device that can provide controlled release of a bioactive agent, it is desirable to have the capability to modulate the shape of the release curve. The time profile of the release of the bioactive agent can range from immediate release where the drug elutes all at once (much like a step function) to an extremely slow, linear (zero order) release, where the drug is evenly released over many months or years. Depending upon the drug and the condition being treated, there are a variety of release profiles that are of interest. The objective of creating medical devices fabricated of biodegradable polymers is to be able to attain the broad range of release profiles that lie between a step function and a low-slope, zero-order release.

In accordance with some aspects of the invention, the shape of the bioactive agent release curve can be modulated by controlling one or more characteristics of the bioactive agent delivery systems, such as the selection of the polymer materials, the relative amounts of polymer components within the system (for example, when the system comprises a blend of more than one polymer material), and the like. In accordance with the invention, the time profile of the release of bioactive agent can be modulated to provide any desired shape, including immediate release where the bioactive agent elutes all at once (much like a step function) to an extremely slow, linear (i.e., zero order) release, where the bioactive agent is evenly released over many months or years. Depending on the bioactive agent and the condition being treated, a variety of release profiles can be achieved. The objective of creating bioactive agent delivery systems with the inventive biodegradable solid polymers is to be able to attain the broad range of release profiles that lie between a step function and a low-slope, zero-order release. Preferably, the polymer materials selected (and the relative amounts of polymers, when more than one polymer material is included in the system) of the bioactive agent delivery system is selected to provide the desired release profile. By controlling the release profiles as described herein, significant improvements can be made to the efficacy of treatment with bioactive agent.

The inventive bioactive agent delivery systems described herein can be designed to control (such as, for example, by limiting or even eliminating) the initial burst of bioactive agent from the coating. The bioactive agent still remaining in the coating after the burst release is then released to the site of action over a longer time period. The shape of the release profile (percentage of bioactive agent released versus time) after the burst can be controlled to be linear or logarithmic or some more complex shape, again depending upon the composition of the blended coating and bioactive agent in the coating composition.

The in vivo release of a bioactive agent can be approximated by observing the in vitro release of the bioactive agent. For example, an implantable device can be fabricated to include a biodegradable coating containing a bioactive agent. The coated implantable device can then be placed in an appropriate solution (for example, a buffer solution such as phosphate buffered saline) for a period of time. During incubation of the device, the solution can be periodically monitored to determine the in vitro release rate of the bioactive agent into the solution. The coated implant is removed from the solution and placed in fresh buffer solution in a new vial at periodic sampling times. Concentration of bioactive agent at each sampling time can be determined in the spent buffer by spectroscopy using the characteristic wavelength for each bioactive agent. The concentration can be converted to a mass of bioactive agent released from the coating using molar absorptivities. The cumulative mass of the released bioactive agent is calculated by adding the individual sample mass after each removal. The release profile is obtained by plotting the amount of released bioactive agent as a function of time. From this determined in vitro release rate, the in vivo release rate can be approximated using known techniques. Typically, the in vitro release rate is slower than an in vivo release rate for the same bioactive agent and biodegradable composition.

The inventive biodegradable compositions exhibit controlled release characteristics, in contrast to a bolus type administration (which includes an initial burst release of bioactive agent) in which a substantial amount of the bioactive agent is made biologically available at one time. For example, in some embodiments, upon contact with body fluids including blood, spinal fluid, lymph, or the like, the biodegradable compositions (formulated as provided herein) can permit a desired amount of initial release of bioactive agent, and subsequently provide a sustained, predictable delivery of the bioactive agent over time. This release can result in prolonged delivery of therapeutically effective amounts of any incorporated bioactive agent. Sustained release will vary in certain embodiments as described in more detail herein.

The phrase “therapeutically effective amount” is an art-recognized term. In some aspects, the term refers to an amount of the bioactive agent that, when incorporated into a polymer matrix of the invention, produces some desired effect at a reasonable benefit/risk ratio applicable to any medical treatment. In some aspects, the term refers to that amount necessary or sufficient to treat a particular disease or disorder, such as age-related macular degeneration (AMD). The phase “prophylactically effective amount” likewise is an art-recognized term. In some aspects, the phrase refers to an amount of bioactive agent that, when incorporated into a polymer matrix of the invention, provides a preventative effect sufficient to prevent or protect an individual from future medical risk associated with a particular disease or disorder. The therapeutically and/or prophylactically effective amount can vary depending upon such factors as the condition being treated (or to be prevented), the particular bioactive agent(s) being administered, the severity of the condition, and the like. In preferred aspects, the therapeutically and/or prophylactically effective amount takes into account the amount of bioactive agent released from the biodegradable composition during any selected time period, particularly the time period during implantation and immediately after the device is emplaced (the initial release). Thus, the therapeutically and/or prophylactically effective amount also applies to the initial release of bioactive agent from the biodegradable composition. By controlling the initial release from the biodegradable composition, preferred embodiments can reduce or eliminate potentially undesirably high amounts of bioactive agent release during early stages after implantation. One of ordinary skill in the art can empirically determine the effective amount of a particular bioactive agent without necessitating undue experimentation.

Aspects of the invention can thus provide one or more advantages, including the ability to provide sustained bioactive agent delivery that can maintain the bioactive agent concentration within a therapeutic window for a prolonged period of time and improve bioactive agent efficacy. Local delivery can reduce bioactive agent dosage, toxicity effects, and other side effects that are typically associated with administration of therapeutics.

Once a therapeutic range has been determined (for example, by a physician), the inventive polymer systems can be adjusted to provide the bioactive agent at a dosage that is within the therapeutic range. The inventive compositions provide improved means to control release of the bioactive agent, thus providing enhanced ability to deliver bioactive agent at desired rates and amounts.

It has further been found that the bioactive agent concentration is a function of the distance between the implant and tissue layers and, thus, the placement of the implant can be customized to provide particular concentrations of bioactive agents with a specific dose/distance relationship to the circumferential or spherical radius of the eluting source. It has been further found that an array of implants can be used in combination to further customize the dose. For example, where the circumferential or spherical radius dose/distance relationship of two or more implants overlap, one or more zones of different bioactive agent concentrations could be achieved.

In some aspects, the inventive biodegradable, implantable devices are fabricated of polymeric materials that can limit initial release of bioactive agent and provide control over the shape of the release profile curves.

The controlled delivery device of the invention can be used to deliver one or more bioactive agents to the eye for the treatment of a variety of ocular conditions such as, for example, retinal detachment; occlusions; proliferative retinopathy; proliferative vitreoretinopathy; diabetic retinopathy; inflammations such as uveitis, choroiditis, and retinitis; degenerative disease (such as age-related macular degeneration, also referred to as AMD); vascular diseases; and various tumors including neoplasms. In yet further embodiments, the controlled delivery device can be used post-operatively, for example, as a treatment to reduce or avoid potential complications that can arise from ocular surgery. In one such embodiment, the controlled delivery device can be provided to a patient after cataract surgical procedures, to assist in managing (for example, reducing or avoiding) post-operative inflammation.

Most, if not all, ophthalmic diseases and disorders are associated with one or more of three types of indications: (1) angiogenesis, (2) inflammation, and (3) degeneration. Based on the indications of a particular disorder, one of ordinary skill in the art can administer any suitable bioactive agent from the three groups at a therapeutic dosage. The following describes some ophthalmic diseases and disorders and a form of treatment therefore. It should be recognized however, that the following is by way of illustration and is not intended to limit the methodologies of the present invention to a particular technique or bioactive agent for treatment of an eye disease or disorder.

Diabetic retinopathy, for example, is characterized by angiogenesis. This invention contemplates treating diabetic retinopathy by delivering one or more anti-angiogenic factors into the sub-retinal space. It also is desirable to co-deliver one or more neurotrophic factors also to the sub-retinal space.

Uveitis involves inflammation. The present invention contemplates treating uveitis by instilling or disposing one or more anti-inflammatory factors in the sub-retinal space.

Retinitis pigmentosa, by comparison, is characterized by retinal degeneration. The present invention contemplates treating retinitis pigmentosa by instilling or disposing one or more neurotrophic factors in the sub-retinal space.

Age-related macular degeneration involves both angiogenesis and retinal degeneration and includes, but is not limited to, dry age-related macular degeneration, exudative age-related macular degeneration, and myopic degeneration. The present invention contemplates treating this disorder by instilling or disposing in the sub-retinal space one or more neurotrophic factors and/or one or more anti-angiogenic factors. More particularly, the methodology contemplates instilling or disposing a corticosteriod in the sub-retinal space.

Glaucoma is characterized by increased ocular pressure and loss of retinal ganglion cells. Treatments for glaucoma contemplated in the present invention include delivery of one or more neuroprotective agents that protect cells from excitotoxic damage. Such agents include N-methyl-D-aspartate (NMDA) antagonists, cytokines, and neurotrophic factors.

The invention will now be described with reference to the following non-limiting examples.

### EXAMPLES

For the Examples, the following procedures applied:

Implantation:

The experiments were performed in accordance the policies in the Guidelines for the Care and Use of Laboratory Animals, the OPRR Public Health Service Policy on the Humane Care and Use of Laboratory Animals (revised 1986), the U.S. Animal Welfare Act, as amended and the Institution's and the Association for Research in Vision and Ophthalmology's (ARVO) policies governing the use of vertebrate animals for research, testing, teaching or demonstration purposes.

### Example 1

Materials Used:

Polycaprolactone (Average Mw 80,000, [—O(CH₂)₅CO—]ₙ, Melt index 125° C./0.3 MPa, Sigma Aldrich Biochemicals, St. Louis, Mo.)
Triamcinolone acetonide (Purity 99%, Mₙ 434.5, C₂₄H₃₁FO₆, Sigma Aldrich Biochemicals, St. Louis, Mo.)
Prednisolone (Purity 99%, C₂₁H₂₈O₅, Mₙ 360.5, Sigma Aldrich Biochemicals, St. Louis, Mo.)
Chloroform (purity 99.8%, CHCl₃, A.C.S. spectroscopic grade, Sigma Aldrich Chemicals)
Ether (purity 99%, Mn 74.12, (C₅H₅)₂O A.C.S. reagent, Sigma Aldrich Chemicals)
Balanced salt solution (Sterile, preservative free, Akorn, Inc., Somerset, N.J.)
Bovine serum albumin (Molecular biology grade, Sigma Aldrich Biochemicals, St. Louis, Mo.)
   Abbreviations:
PCL: polycaprolactone biodegradable filament
TA: triamcinolone
PCL/TA: biodegradable triamcinolone loaded polycaprolactone filaments
   Filament Preparation:

The filaments used in the example were prepared as follows. PCL was solubilized in chloroform at 35° C. overnight under continuous stirring conditions. Triamcinolone acetonide (TA) was then added to the solution in a polymer/drug weight ratio (w_{P}/w_{D}) of 70:30, 60:40 or 50:50. Once the solution became homogeneous, it was poured onto an evaporating tray and left in a fume hood for 72 hours to solidify. The white solid-form sheath of the TA loaded PCL was rolled into a tight column and packed into a 10 mL syringe. The syringe was heated to 80° C. in a water bath to ensure even heat distribution and to prevent high localized temperatures that could damage the drug or polymer. Although the polymer was not fully in the melt state, the temperature was sufficiently high to initiate the transition of this semi-crystalline closed packed macromolecular polymer to a sufficiently viscous state to be extruded. Additionally, it was noted that drug crystals within the polymer acted as a “flow enhancing” plasticizer when comparing the process to a PCL only filament extrusion.

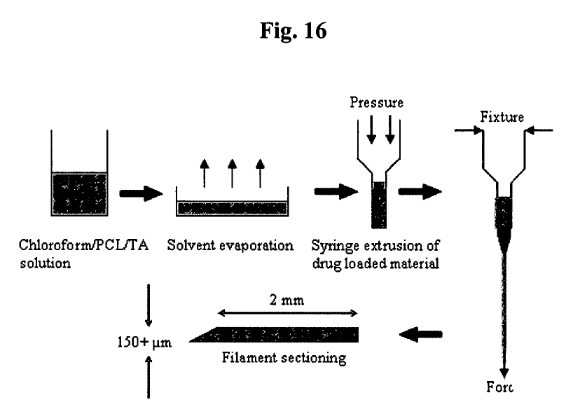

Once the syringe reached 80° C. it was rapidly removed from the water bath and 1 cm of material was extruded from it. The extruded material was subsequently drawn to a filament by imparting a tensile force (FIG. 16). For the 70:30, 60:40 or 50:50 w_{P}/w_{D} formulations, ˜150 μm filament diameters were achieved by a drawing length of approximately 20, 15 and 10 cm, respectively, while ˜300 μm filament diameters were achieved by a drawing length of approximately 15, 10 and 5 cm, respectively. The formulation with the highest drug load (50:50 w_{P}/w_{D}) broke more frequently during the drawing process. The drawn filament cooled rapidly and could be subsequently cut under a microscope to the desired implantation length.

Filaments without drug were also prepared by directly inserting the PCL pellets into the syringe, heating them to 80° C. and then extruding and drawing in a similar manner to that previously described.

Six pigmented rabbits underwent fluorescein angiography, color fundus photography, and optical coherence tomography (Zeiss Model 3000, Germany) at baseline and 4 weeks after implantation. The rabbits were subdivided into the following groups:
Group 1: 2 rabbits with PCL only filaments (PCL, Rabbits 1 and 2);
Group 2: 4 rabbits with PCL/TA 60:40 (w_{P}/w_{D}) filaments (Rabbits 3-6).

Both groups underwent standard pars plana vitrectomy, and insertion of the drug delivery device into the subretinal space. Briefly, animals were anesthetized with an intramuscular injection of 0.3 mL of ketaminehydrochloride (100 mg/mL; Fort Dodge Lab., Iowa) and 0.1 mL of xylazine hydrochloride (100 mg/mL; Miles Inc, USA) per kilogram of body weight. Pupils were dilated with 1 drop each of 2.5% phenylephrine and 1% tropicamide. A 3-mm peritomy was made at the superotemporal and superonasal quadrant of the right eye. Sclerotomies were created with a 20-gauge microvitreoretinal blade 1 to 2 mm posterior to the limbus in the superotemporal and superonasal quadrants. An infusion line was inserted and sutured through the superonasal sclerotomy and a vitreous cutter (Bausch & Lomb, USA) was inserted through the superotemporal sclerotomy. The vitreous cutter and infusion line were used to perform a 2-port core vitrectomy. The illumination provided by the operating microscope (Zeiss, Germany) was sufficient for the operation.

Using intraocular microscopic forceps (Bausch & Lomb, USA), the filaments were inserted in the subretinal space through a small self-sealing retinotomy. The beveled tip of the implant allowed easy insertion through the retina. The filament was left in position and the forceps was withdrawn from the eye. No laser retinopexy was applied to seal the retinal breaks. The infusion line was removed and the sclerotomies and conjunctival openings were closed using Vycril 7-0 (Ethicon, USA). During week 4, all rabbits underwent fundus examination and were then sacrified under anesthesia using an intracardiac injection of pentobarbital sodium (Anpro Pharmaceuticals, Oyster Bay, N.Y.).

Elution, Drug Extraction and Histology:

In Vitro Elution

For in vitro drug elution characterization, drug-loaded PCL filaments were prepared according to Table 1.

TABLE 1
In vitro sample parameters

| | Formulation | Diameter | Length |
|---|---|---|---|
| Sample | PCL/TA | (μm) | (mm) |
| 1 | 70:30 | 210 | 30 |
| 2 | 70:30 | 210 | 30 |
| 3 | 70:30 | 250 | 30 |
| 4 | 70:30 | 250 | 30 |
| 5 | 70:30 | 360 | 30 |
| 6 | 60:40 | 150 | 30 |
| 7 | 60:40 | 150 | 30 |
| 8 | 60:40 | 320 | 30 |
| 9 | 60:40 | 320 | 30 |
| 10 | 50:50 | 150 | 30 |
| 11 | 50:50 | 150 | 30 |
| 12 | 50:50 | 320 | 30 |
| 13 | 50:50 | 320 | 30 |

Each filament was placed in a 15 mL capped tube containing 10 mL of a 1% bovine serum albumin (BSA)/balance salt solution (BSS). Tubes were incubated at 37° C. in a shaking water bath (100 rpm). At ech time increment of 2, 4, 8, 24, 72, 168, 336, 504 and 672 hours, the filaments were removed from the BSS/BSA solution and placed into a new 10 ml BSS/BSA solution.

After the final time period, the filaments were removed from the BSS/BSA solution and placed in tubes containing 2 mL of ether for complete extraction of the remaining TA. Ether (2 mL) and a 50 μL of internal standard (prednisolone 2 mg/ml) were added to the remaining BSS/BSA solutions. Each solution was vortexed for 2 min and then centrifuged for 3 min at 10,000 rpm to separate the ether and BSS/BSA phases. The top layer ether phase was removed using a glass syrine and added to a 2 mL capped microtube for solvent evaporation in fume hood. Following complete evaporation, 1 mL of 60% methanol was added to the microtube and vortexed. The solution was then transferred to a 1 mL glass shell high performance liquid chromatography (HPLC) vial for analysis.

In Vivo Elution

Two rabbits (PCL/TA 60:40 filaments) were used for analysis of in vivo drug elution. Rabbits were anesthetized prior to the collection of aqueous (˜0.3 mL) and blood into lithium heparin tube (2 mL). Rabbits were then euthanized and the eyes enucleated. The implanted device and surrounding tissues (sclera, choroid, retina, lens, and vitreous) were dissected and separated into 2 mL micro tubes. Individual tissue was weighed and then homogenized in 0.5 mL BSS by sonication (1-2 pulse/sec at 50% power). Once completed, samples were enriched with 50 μL internal standard (prednisolone 2 mg/ml) and vortexed. TA was extracted from the tissue sample by adding ether (0.5 mL), vortexing and centrifuging at 10,000 rpm for 10 min. The top ether layer was removed and placed in a new 2 mL microtube for evaporation and substitution of the solvent for methanol as previously described in the in vivo study.

A Millennium high performance liquid chromatograph (Waters Corp., USA) equipped with a 515 pump, 2996 photodiode array detector and 717-plus autosampler injector was used in this study to process the in vitro and in vivo samples. The Millennium software provided with the high performance liquid chromatograph (HPLC) was used for integration of chromatographic peaks. The solvents were linked to an in-line degasser. The samples were injected into reverse phase HPLC system consisting of stationary phase of Nova-Pak C18 column (3.9×150 mm) and Nova-Pack guard column (Waters Corp., USA); and an isocratic mobile phase of 60% methanol. The peaks of TA and prednisolone were eluted at a flow rate of 1 mL/min with detection at 245 nm. Parallel 50 μL of prednisolone was chromatogramed under the same HPLC condition to determine the extraction efficiency of TA. Further, the co-chromatography technique was adopted to validate the identification of both compounds. The calculation of TA concentration was based on the area peaks and percentage recovery of prednisolone. The HPLC condition separated the peaks of triamcinolone and prednisolone with good resolution. The retention time of prednisolone was 3.46 minutes while that of triamcinolone was 5.2 minutes.

Histology

The eyes of the four remaining rabbits (2 rabbits with PCL only filaments; 2 rabbits with PCL/TA 60:40 filaments) were enucleated and fixed in 4% paraformaldehyde for 24 hours and then Bouin's fixative for a further 24 hours. The specimens were then embedded in paraffin, sectioned, and hematoxylin and eosin (H & E) stained under standard histology laboratory conditions.

Results:

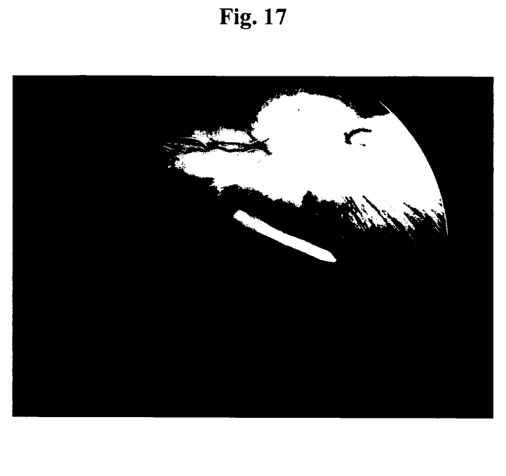

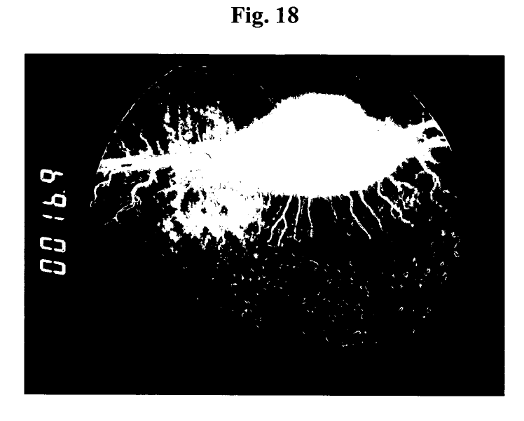

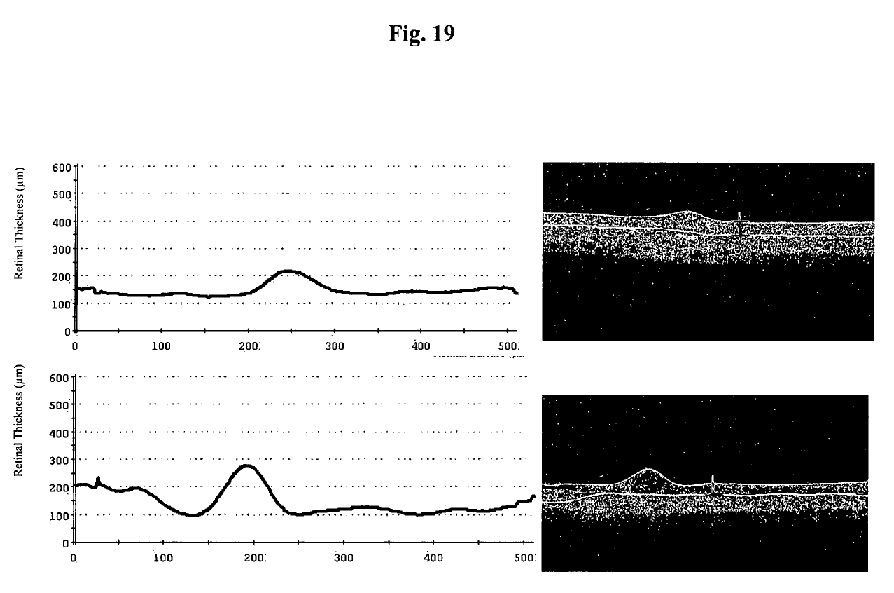

Clinical examination using slit-lamp and indirect ophthalmoscopy at 1, 2, 3 and 4 weeks showed that there was no detectable accumulation of subretinal fluid, exudates, hemorrhage or fibrosis surrounding the device at any of the follow up points. Fundus photography showed that the filament maintained its position without signs of inflammation or migration, as shown in FIG. 17 for a representative rabbit. Fluorescein angiography demonstrated the absence of vascular leakage, pooling, retinal pigmented epithelium (RPE) abnormalities, or fibrosis at any of the follow-up points for a representative rabbit, as shown in FIG. 18. Optical coherence tomography revealed the successful placement of the implant in the subretinal space of all the rabbit eyes, as shown in FIG. 19.

The topographical effect of using different filament diameters (150 μm vs. 320 μm) can also be seen in FIG. 19 by the comparative increase in retinal thickness at the site of the implant. No abnormalities were reported from increasing the filament diameter. An increase in the filament diameter merely resulted in a slightly more demanding surgical procedure and a larger area of cellular disruption.

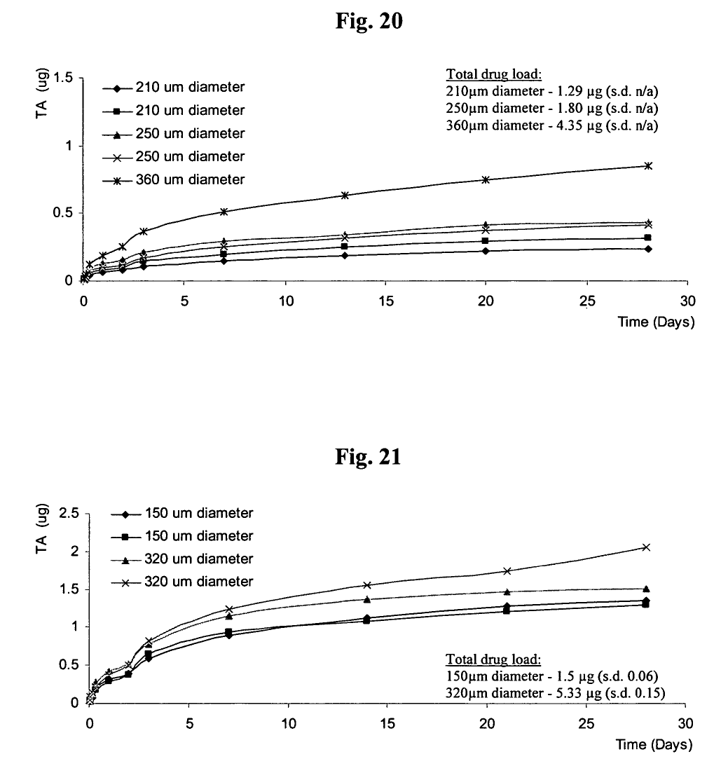

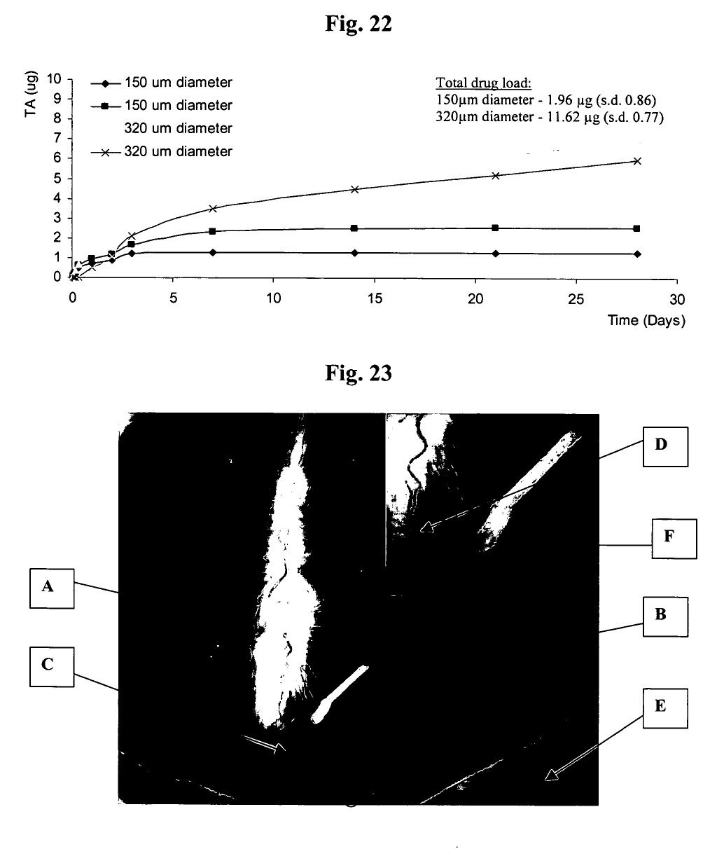

The in vitro elution rates for the different polymer-drug ratios and geometries into a BSS/BSA (1%) solution are shown in FIGS. 20, 21 and 22. In general, the elution rates showed an early burst phase followed by a late first order phase. Without being bound by a particular theory, it is believed that the initial early rapid-release phase is attributed to the absorption of drug crystals in the surface to subsurface region of the filament into the medium, preceding diffusion from the polymer core. This initial burst may be particularly useful if it is desired to rapidly achieve local therapeutic dosage. For each of the different polymer-drug ratios, increasing the filament diameter or drug:polymer ratio resulted in an increase in the amount of drug eluted. Without being bound by theory, it is believed that this change results from the increased drug content and/or eluting surface area. For the larger (˜300 μm) filaments, increasing the ratio of drug in the formulation from PCL/TA 70:30 to 50:50 also increased the drug elution rate, while a drug dumping effect occurs if both the drug ratio is high (PCL/TA 50:50) and the filament diameter is small, as shown in FIG. 22. In this latter case, total drug release had occurred during the initial burst, and the rate of TA absorption by the subretinal tissue was most likely a limiting factor. The near superimposition of all the elution profiles during the first few hours of each study also indicated that it was the rate of TA absorption that was the limiting step during the first stage of elution. Polycaprolactone is hydrophobic and impermeable to enzyme diffusion; therefore swelling, bulk diffusion, or degradation is unlikely in a bodily environment. Without intending to be bound by a particular theory, the TA elution profile that occurs after the initial surface to subsurface event is believed to be the result of a microporous drug boundary layer being formed and moving depper toward the core as the TA crystals are progressively absorbed by the body. As a result, the lower the drug loading, the smaller the polymer porosity formed during drug absorption and the lower the rate of TA elution.

Illustrative images (optical and histology staining) of implanted filaments are shown in FIGS. 23 through 25. The size of the retinotomy shown in the optical images is approximately 500 μm. However, smaller sized retinotomies are possible with the use of custom implantation tools.

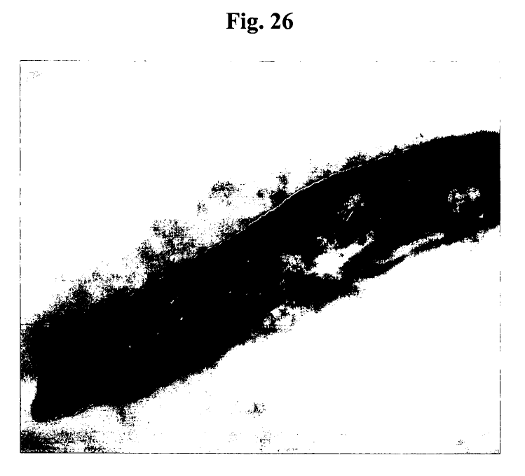

Compared with the initial implant, the explanted filaments at four weeks post surgery had a somewhat more fibrous polymer microstructure, as shown in FIG. 26, than the initial implant. In some studies, only a flaky fibrous/porous polymer microstructure remained once the entire drug was extracted from the device during the in vitro elution studies. The molecular number selected for this polymer was at the high end (M_{w} 80,000) of the commercially available range. PCL degrades by a reduction in M_{w}, so a longer degradation time is expected with this high M_{w}. There was no indication that polymer degradation had begun during the follow-up period.

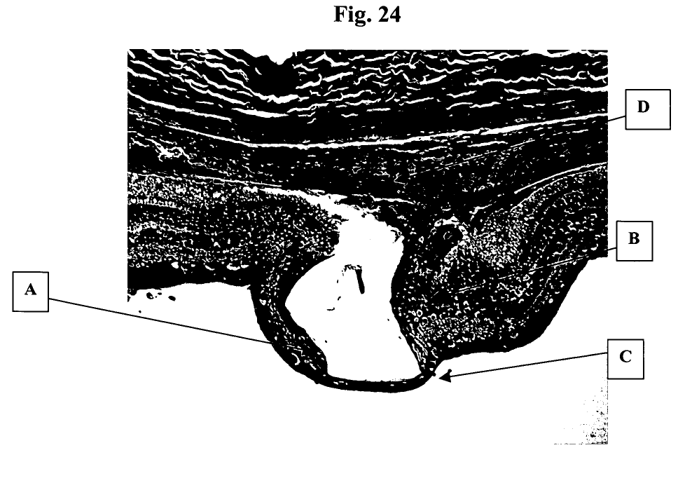

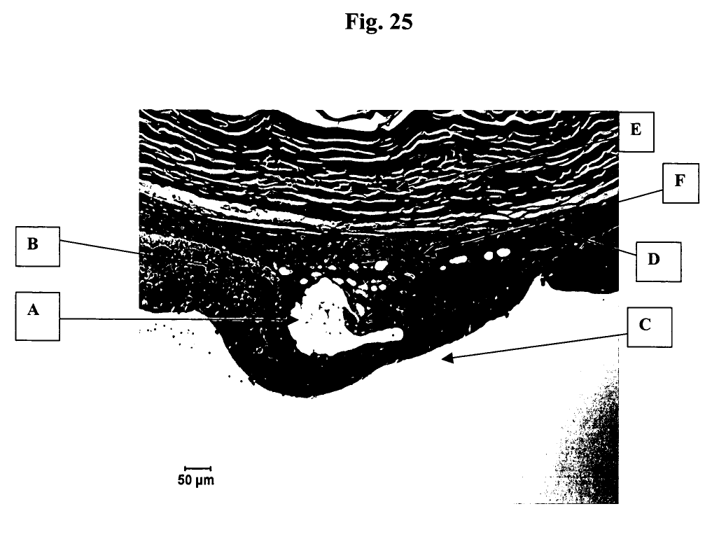

Histology revealed that the implants, whether drug loaded or not, were encapsulated by one or two cell layers that did not appear fibrotic in nature, as shown in FIGS. 24 and 25. The nerve fiber layer (ganglion axles) above the filament appeared intact, while the support cells immediately over the filament location are clearly absent in the PCL only implant and somewhat disrupted and thinned in the TA/PCL implanted eye. The Bruch's membrane appeared intact but there was evidence of thinning and disruption of the outer nuclear and RPE layers adjacent to the filament. Due to the lack of inflammatory response, PCL demonstrated excellent compatibility with this tissue region and the bulk of the observed cellular changes were attributed to the mechanical damage during the implantation. Other factors such as the impact of interfering with the nutritional source of these outer cellular layers may also play a role in these cellular changes.

It has been found that PCL degrades by random hydrolytic chain scission in subdermally implanted rabbits. The degradation initially manifests by a progressive reduction in molecular weight as the chain scission reactions propagate. However, it has also been shown that the physical weight of PCL does not change until the molecular weight has fallen to 5000—that is, there is no weight loss during the first phase of the degradation (Pitt C G. Poly ε caprolactone and its copolymers, In Chassin M Langer R, editors, Biodegradable polymers as drug delivery systems, New York: Dekker; 1990. p 71-119). Thus, phagocytosis and metabolism of small PCL fragments will not begin until the final phase of the degradation process. Further, PCL has shown excellent biocompatibility during the one-month follow up period.

The PCL/TA drug delivery system showed less disruption to the RPE layer and less tissue layer thinning in the adjacent regions of the implant than the PCL only filament, as shown in FIGS. 24 and 25. However, it is difficult to conclude whether this could be attributed to the anti-inflammatory effect of the steroid or was simply due to variability in surgical procedure and positioning. The region of retinal cell layers disruption where the implant resides extends for approximately 300 μm in width and 2000 μm in length. It has been found that the nerve fiber layer remains intact over the implant, but is disrupted at the site of the retinotomy. Thus, only a very focal region of vision loss is expected and one that is certainly less invasive than laser photocoagulation therapy.

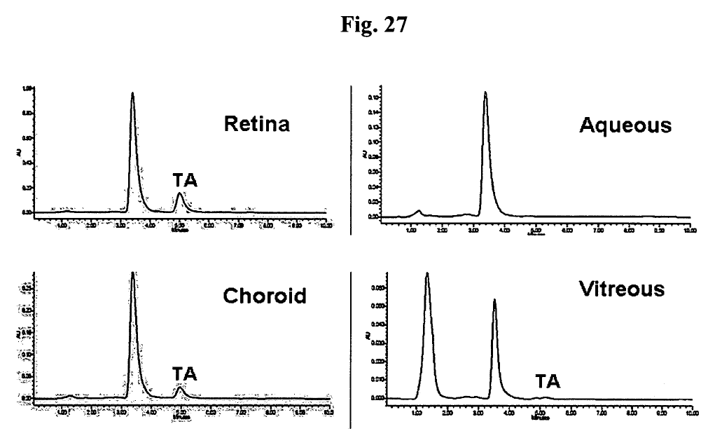

HPLC confirmed the presence of TA four weeks after the implant in the posterior tissue samples (FIG. 27). TA was not detected in the anterior structures or the blood. HPLC peaks for TA are marked on the graphs shown in FIG. 27. The additional peaks present indicate the internal standard prednisolone.

Based upon this initial investigation, it has been demonstrated that PCL has at least a one month elution capability with TA. Drug levels in the tissue were shown to be localized to the posterior eye segment. Histology showed no indication of inflammatory response from the presence of PCL. Minor mechanical damage from the insert was observed and is believed to be the leading cause of changes in the cellular layers and structures PCL encapsulation was also evident and is expected for implanted materials.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

We claim:

1. A medical device comprising an implant configured for placement in a posterior region of the eye, the implant comprising one or more bioactive agents, and a biodegradable amphiphilic block copolymer including hydrophilic blocks and hydrophobic blocks.

2. The medical device according to claim 1 wherein the implant is configured for placement in a subretinal area of the eye.

3. The medical device according to claim 2 wherein the implant is tapered at a proximal end, a distal end, or both the proximal and distal ends.

4. The medical device according to claim 2 wherein the implant is configured to be positioned in one or more tissue layers above a choroid but below a nerve fiber layer of an eye.

5. The medical device according to claim 2 wherein the implant has a total diameter of no greater than 1000 μm and a length of no greater than 6 mm.

6. The medical device according to claim 2 wherein the implant has a bioactive agent elution rate of at least 0.0001 μg per day.

7. The medical device according to claim 2 wherein at least 5% of the bioactive agent released from the implant is delivered to the retina.

8. The medical device according to claim 1 wherein the hydrophilic blocks comprise polyalkylene glycol.

9. The medical device according to claim 8 wherein the polyalkylene glycol is selected from the group polyethylene glycol, polypropylene glycol, and polybutylene glycol.

10. The medical device according to claim 9 wherein the polyalkylene glycol is selected from the group polyethylene glycol terephthalate, polypropylene glycol terephthalate, and polybutylene glycol terephthalate.

11. The medical device according to claim 8 wherein the polyalkylene glycol blocks comprise polymers having a formula:
—OLO—CO—R—CO—
wherein L is a divalent organic radical remaining after removal of terminal hydroxyl groups from a poly(oxyalkylene)glycol, O represents oxygen, C represents carbon, and R is a substituted or unsubstituted divalent radical remaining after removal of carboxyl groups from a dicarboxylic acid.

12. The medical device according to claim 8 wherein the hydrophobic blocks comprise aromatic polyester formed from an alkylene glycol having 2 to 8 carbon atoms and a dicarboxylic acid.

13. The medical device according to claim 12 wherein the polyester is selected from the group polyethylene terephthalate, polypropylene terephthalate, and polybutylene terephthalate.

14. The medical device according to claim 12 wherein the aromatic polyester blocks comprise polymers having a formula:
—OEO—CO—R—CO—
wherein E is an organic radical selected from the group of substituted or unsubstituted alkylene radical shaving 2 to 8 carbon atoms, and a substituted or unsubstituted ether moiety, O represents oxygen, C represents carbon, and R is a substituted or unsubstituted divalent aromatic radical.

15. The medical device according to claim 1 wherein the amphiphilic block copolymer comprises polyethylene glycol/polybutylene terephthalate block copolymer.

16. The medical device according to claim 1 further comprising a core, and wherein the biodegradable amphiphilic block copolymer and one or more bioactive agents are provided as a coating on a surface of the core.

17. The medical device according to claim 16 wherein the coating is provided on a portion of the core surface.

18. The medical device according to claim 17 wherein the coating is provided on an intermediate portion of the core.

19. The medical device according to claim 16 wherein the coating includes proximal a transition segment, a distal transition segment, or both a proximal and a distal transition segment.

20. The medical device according to claim 16 wherein the core is fabricated of a nondegradable material.

21. The medical device according to claim 20 wherein the nondegradable material is selected from titanium alloys, nickel-cobalt base alloys, stainless steel, cobalt-chromium alloys, and biodegradable magnesium alloys.

22. The medical device according to claim 20 wherein the nondegradable material comprises one or more polymers selected from poly(methyl methacrylate) and silicone.

23. The medical device according to claim 22 wherein the nondegradable material includes one or more bioactive agents.

24. The medical device according to claim 16 wherein the core is fabricated of a biodegradable material.

25. The medical device according to claim 24 wherein the biodegradable material comprises an amphiphilic block copolymer comprising hydrophilic blocks and hydrophobic blocks.

26. The medical device according to claim 25 wherein the biodegradable material is selected from polyglycolic acid, polydioxanone, surgical gut, polylactic acid, polyglyconate, polyglactin, and polyglecaprone.

27. The medical device according to claim 1 wherein the bioactive agent is selected from antiproliferative agent, anti-inflammatory agent, anti-angiogenic agent, antibiotic, neurotrophic factor, or a combination of any two or more of these.

28. The medical device according to claim 1 wherein the implant comprises:
a nonlinear body member having a direction of extension, a longitudinal axis along the direction of extension, and a proximal end and a distal end,
wherein at least a portion of the body member deviates from the direction of extension,
and wherein the body member includes the one or more bioactive agents, and the polymer matrix comprising a biodegradable amphiphilic block copolymer comprising hydrophilic blocks and hydrophobic blocks.

29. The medical device according to claim 28 wherein the body member is coil-shaped.

30. The medical device according to claim 28 wherein a cap is positioned at the proximal end of the body member.

31. The medical device according to claim 28 wherein the body member includes a lumen.

32. The medical device according to claim 28 wherein the body member includes a core.

33. The medical device according to claim 32 wherein the core is fabricated of a nondegradable material.

34. The medical device according to claim 33 wherein the nondegradable material is selected from titanium alloys, nickel-cobalt base alloys, stainless steel, cobalt-chromium alloys, and biodegradable magnesium alloys.

35. The medical device according to claim 33 wherein the nondegradable material comprises one or more polymers selected from poly(methyl methacrylate) and silicone.

36. The medical device according to claim 35 wherein the nondegradable material includes one or more bioactive agents.

37. The medical device according to claim 32 wherein the core is fabricated of a biodegradable material.

38. The medical device according to claim 37 wherein the biodegradable material is selected from polyglycolic acid, polydioxanone, surgical gut, polylactic acid, polyglyconate, polyglactin, and polyglecaprone.

39. The medical device according to claim 28 wherein the device is removable from the eye after a desired treatment.

40. A method of making a device for controlled release of bioactive agent to a posterior region of an eye, the method comprising steps of providing a biodegradable amphiphilic block copolymer comprising hydrophilic blocks and hydrophobic blocks, combining the biodegradable amphiphilic block copolymer with one or more bioactive agents, and forming the copolymer with bioactive agent into an implant configured for placement in ocular tissues within the posterior region of the eye.

41. The method according to claim 40 wherein the step of forming the copolymer with bioactive agent into an implant comprises forming the copolymer with bioactive agent into a filament, rod, C-shaped implant, coil, film, ribbon, block, disc, or pellet for placement in a subretinal area of the eye.

42. The method according to claim 40 wherein the step of forming the copolymer with bioactive agent into an implant comprises providing a core and providing bioactive agent and copolymer to a surface of the core.

43. The method according to claim 40 wherein the step of forming the copolymer into an implant comprises forming the copolymer with bioactive agent into a nonlinear body member having a direction of extension, a longitudinal axis along the direction of extension, and a proximal end and a distal end, wherein at least a portion of the body member deviates form the direction of extension, the implant configured for intraocular placement within an eye.

44. The method according to claim 40 wherein the step of forming the copolymer with bioactive agent into an implant comprises providing a core comprising a nonlinear body member having a direction of extension, a longitudinal axis along the direction of extension, and a proximal end and a distal end, wherein at least a portion of the body member deviates form the direction of extension, and providing the copolymer with bioactive agent to a surface of the core.

45. A method for delivery of bioactive agent to ocular tissue within a patient in a controlled manner, the method comprising steps of implanting a device in a posterior region of the patient's eye, the device comprising a body member fabricated of one or more bioactive agents and a biodegradable amphiphilic block copolymer comprising hydrophilic blocks and hydrophobic blocks.

46. The method according to claim 45 further comprising a step of allowing the device to remain in the patient for a selected period of time, wherein the device is configured to degrade upon implantation for a degradation period, and wherein bioactive agent is released in a controlled manner for a release period, the release period constituting at least a portion of the degradation period.

47. The method according to claim 45 wherein release period comprises 50% or less of the degradation period.

48. The method according to claim 45 wherein the release period comprises 25% or less of the degradation period.

49. The method according to claim 48 wherein the degradation period is in the range of 0.5 to 2 years.
